SEQUENCE LISTING (11 sequences)

SEQ ID NO: 1 - DNA, 89 bp, Artificial Sequence
  Description of Artificial Sequence Synthetic oligonucleotide
aaaaaggtctaagtggagcccttcgagtgttagaagcttgtgacactcggagggcttcacttgggcccggtgtttcgtcctttccacaa

SEQ ID NO: 2 - DNA, 93 bp, Artificial Sequence
  Description of Artificial Sequence Synthetic oligonucleotide
aaaaaaaacatccgactgcgactcctccatagcagcaagcttcctgccatggaggagtcacagtcggatatcggtgtttcgtcctttccacaa

SEQ ID NO: 3 - DNA, 23 bp, Artificial Sequence
  Description of Artificial Sequence Synthetic primer
caccgatttaggtgacactatag

SEQ ID NO: 4 - DNA, 93 bp, Artificial Sequence
  Description of Artificial Sequence Synthetic primer
aaaaaaatcacactcagaactcacaccagaaggctcaagcttcaaccttctgatgtaagttctgagtgtgacggtgtttcgtcctttccacaa

SEQ ID NO: 5 - DNA, 93 bp, Artificial Sequence
  Description of Artificial Sequence Synthetic primer
aaaaaaaagagtagtcttcagcctcgcagtaatcacaagcttctgattaccgcgaggctgaagaccaccctcggtgtttcgtcctttccacaa

SEQ ID NO: 6 - DNA, 93 bp, Artificial Sequence
  Description of Artificial Sequence Synthetic primer
aaaaaagagatagggaccccaagcctgagctggagcaagcttcccccagctcaggcctggggcccctacctcggtgtttcgtcctttccacaa

SEQ ID NO: 7 - DNA, 93 bp, Artificial Sequence
  Description of Artificial Sequence Synthetic primer
aaaaaagagagccgccctcctagcatgcgcaggcccaagcttcggcccgcgcacgccaggagggcagctctcggtgtttcgtcctttccacaa

SEQ ID NO: 8 - DNA, 93 bp, Artificial Sequence
  Description of Artificial Sequence Synthetic primer
aaaaaagggactccaagatccctgagtaagaggagcaagcttcctccccttacccagggatcctggagccccggtgtttcgtcctttccacaa

SEQ ID NO: 9 - DNA, 93 bp, Artificial Sequence
  Description of Artificial Sequence Synthetic primer
aaaaaagggagggctaaggaccgtccgagcacgagcaagcttccccgcgcccggacggtcctcagccctcccggtgtttcgtcctttccacaa

SEQ ID NO: 10 - DNA, 37 bp, Artificial Sequence
  Description of Artificial Sequence Synthetic primer
ccgctcgaggccaccatggccaagcaaccttctgatg

SEQ ID NO: 11 - DNA, 33 bp, Artificial Sequence
  Description of Artificial Sequence Synthetic primer
ccggaattctcaatgccttctccataccagacg

(19) United States
(12) Patent Application Publication
(10) Pub. No.: US 2009/0217404 A1
(43) Pub. Date: Aug. 27, 2009

(54) Cell-based RNA interference and related methods and compositions

(76) Inventors: Scott W. Lowe, Cold Spring Harbor, NY (US); Michelle A. Carmell, Dorchester, MA (US); Gregory J. Hannon, Huntington, NY (US); Patrick Paddison, Oyster Bay, NY (US); Jack Zilfou, Allentown, PA (US); Jordan Fridman, Newark, DE (US); Ross Dickins, Carlton (AU); Michael Hemann, Cambridge, MA (US); Thomas A. Rosenquist, Sound Beach, NY (US); Prem Premsrirut, New York, NY (US)

Correspondence Address: WilmerHale/Cold Spring Harbor Laboratory, 399 Park Avenue, New York, NY, 10022, US

(21) Appl. No.: 12/072,124

(22) Filed: Feb. 23, 2008

Related U.S. Application Data
(63) Continuation-in-part of application No. 10/524,690, filed on Feb. 27, 2008.
Application No. 10/524,690 is a continuation-in-part of application No. 11/893,611, filed on Aug. 15, 2007.
(60) Provisional application No. 60/414,605, filed on Sep. 27, 2002.
(60) Provisional application No. 60/838,025, filed on Aug. 15, 2006.

(51) Int. Cl. A01K 67/027; C12Q 1/68; C12N 5/00; C12N 5/06; C12N 5/08; C07H 21/00

(52) U.S. Cl. 800/18; 435/6; 435/325; 435/354; 435/366; 536/23.1; 800/14

(57) ABSTRACT

The invention provides, among other things, methods for performing RNA interference (RNAi) in stem cells (such as embryonic stem cells) and methods for using such stem cells in vivo. The invention also provides various animal models based on conditional/inducible, reversible, tissue-specific/spacial, and/or developmental stage-specific/temporal RNAi of certain target genes, which animal model may be useful for, e.g., drug target identification and/or validation.

### RELATED APPLICATIONS

This application is a continuation-in-part of U.S. application Ser. No. 10/524,690, filed on Feb. 15, 2005, which is a national stage filing under 35 U.S.C. § 371 of International Application No. PCT/US2003/030901, filed on Sep. 29, 2003, which claims priority from U.S. Provisional Application No. 60/414,605, filed on Sep. 27, 2002.

This application is also a continuation-in-part of U.S. application Ser. No. 11/893,611, filed on Aug. 15, 2007, which claims the benefit of the filing date under 35 U.S.C. § 119(e) of U.S. Provisional Application No. 60/838,025, filed on Aug. 15, 2006.

The specifications of each of the above-referenced applications are incorporated by reference herein.

International Application PCT/US2003/030901 was published under PCT Article 21(2) in English.

### STATEMENT REGARDING FEDERAL FUNDING

Work described herein was funded, in whole or in part, by grants CA13106 and CA87497 from NCI and a grant R01-GM62534 from NIH. The United States Government has certain rights in the invention.

### BACKGROUND

“RNA interference”, “post-transcriptional gene silencing”, “quelling”—these different names describe similar effects that result from the overexpression or misexpression of transgenes, or from the deliberate introduction of double-stranded RNA into cells (reviewed in Fire A (1999) Trends Genet 15: 358-363; Sharp P A (1999) Genes Dev 13: 139-141; Hunter C (1999) Curr Biol 9: R440-R442; Baulcombe D C (1999) Curr Biol 9: R599-R601; Vaucheret et al. (1998) Plant J 16: 651-659). The injection of double-stranded RNA into the nematode C. elegans, for example, acts systemically to cause the post-transcriptional depletion of the homologous endogenous RNA (Fire et al. (1998) Nature 391: 806-811; and Montgomery et al. (1998) PNAS 95: 15502-15507). RNA interference, commonly referred to as RNAi, offers a way of specifically and potently inactivating a cloned gene, and is proving a powerful tool for investigating gene function.

Significant breakthroughs in RNAi technology have permitted the application of this technique to the cells of higher eukaryotes, including humans and other mammals. However, RNAi techniques have not been used to stably transfect mitotically active cells, such as stem cells, tumor cells or certain differentiated cells, in a manner that permits the reconstitution of tissues, organs and whole organisms that comprise cells affected by an RNAi construct.

The invention is intended to address these and other shortcomings in the field of RNAi technology.

### SUMMARY OF THE INVENTION

In certain aspects, the invention provides systems which use RNA interference to stably and specifically target and decrease the expression of one or more target genes in cells, such that the cells may be introduced into a living organism and propagated without significant loss of the RNA interference effect. In certain aspects the invention provides methods for modifying cells ex vivo with a short hairpin RNA (shRNA) expression construct to achieve an RNA interference effect and introducing the cells into a subject. In certain aspects the invention provides vectors and methods for controlling the temporal and spatial expression of a shRNA construct in cells and organisms.

In one aspect, the invention provides methods for introducing into a subject a population of stem cells having partial or complete loss of function of a target gene, the method comprising: a) introducing a nucleic acid construct encoding an shRNA into stem cells to generate transfected stem cells, wherein the shRNA is complementary to a portion of the target gene; and b) introducing the transfected stem cells into the subject, wherein the transfected stem cells propagate within the subject and retain partial to complete loss of function of the target gene. Optionally, the target gene participates in a disease process in the subject. The transfected cells may replace a population of diseased cells in the subject; the diseased cells may be ablated prior to administration of the cells. In certain embodiments, the shRNA construct is expressed constitutively. In other embodiments, shRNA construct expression is conditional. For example, expression of the shRNA may conditional on the presence or absence of a substance administered to the subject. shRNA expression may be cell lineage specific, either because the shRNA expression is driven by a lineage specific promoter or because introduction of the shRNA construct is limited to cells of a particular lineage. Optionally, the cells are stem cells, such as hematopoietic stem cells or embryonic stem cells. In certain embodiments, the transfected stem cells are cultured so as to generate a population of further differentiated transfected stem cells for introduction into the subject.

In certain aspects the invention provides vectors for stably or controllably introducing shRNA constructs into cells. Such vectors may be retroviral vectors, such as lentiviral vectors.

In certain aspects, the invention provides methods for introducing into a subject a population of differentiated cells having partial or complete loss of function of a target gene, the method comprising: a) introducing a nucleic acid construct encoding an shRNA into stem cells to generate transfected stem cells, wherein the shRNA is complementary to a portion of the target gene; b) culturing the transfected stem cells to generate transfected differentiated cells having partial or complete loss of function of a target gene; and c) introducing the transfected differentiated cells into the subject, wherein the transfected differentiated cells retain partial to complete loss of function of the target gene.

In certain aspects, the invention provides methods of treating a disease associated with the expression of a target gene in a population of cells, the method comprising: a) introducing a nucleic acid construct encoding an shRNA into stem cells to generate transfected stem cells, wherein the shRNA is complementary to a portion of the target gene; and b) introducing the transfected stem cells into the subject,

In further aspects, the invention provides non-human mammals comprising a population of stem cells comprising a nucleic acid construct encoding an shRNA, or progeny cells thereof, wherein the cells exhibit partial to complete loss of function of a target gene.

In one aspect, the invention provides compositions formulated for administration to a human patient, the composition comprising: a) a stem cell comprising a nucleic acid construct encoding an shRNA, wherein the shRNA is complementary to at least a portion of a target gene, and wherein the cells exhibit partial to complete loss of function of a target gene; and b) a pharmaceutically acceptable excipient.

In certain aspects, the invention provides methods for identifying a gene that affects the sensitivity of tumor cells to a chemotherapeutic agent, the method comprising: a) introducing into a subject a transfected stem cell comprising a nucleic acid construct encoding an shRNA, wherein the shRNA is complementary to at least a portion of a target gene, wherein the transfected stem cell exhibits decreased expression of the target gene, and wherein the transfected stem cell gives rise to a transfected tumor cell in vivo; b) evaluating the effect of the chemotherapeutic agent on the transfected tumor cell. Optionally, evaluating the effect of the chemotherapeutic agent on the transfected tumor cell comprises: administering the chemotherapeutic agent to the subject and measuring the quantity of tumor cells derived from the transfected stem cell. A method may further comprise comparing the quantity of tumor cells derived from the transfected stem cell to the quantity of tumor cells derived from the transfected stem cell in a control subject that has not received the chemotherapeutic agent.

In certain aspects, the invention provides methods for identifying a gene that affects the sensitivity of tumor cells to a chemotherapeutic agent, the method comprising: a) introducing into a subject a plurality of transfected stem cells, wherein each transfected stem cell comprises a nucleic acid construct comprising a representative shRNA of an shRNA library, and wherein a representative shRNA of an shRNA library is complementary to at least a portion of a representative target gene, wherein a plurality of the transfected stem cells exhibits decreased expression of a representative target gene, and wherein a plurality of the transfected stem cells gives rise to transfected tumor cells in vivo; b) administering a chemotherapeutic agent; and c) identifying representative shRNAs that are enriched or depleted by treatment with the therapeutic agent. In a further aspect the invention provides a method of administering a chemotherapeutic agent to a patient, the method comprising: a) administering the chemotherapeutic agent; and b) administering a nucleic acid that causes RNA interference of a gene that is associated with chemotherapeutic resistance.

In certain aspects, the invention provides a barcoded shRNA library comprising a plurality of representative shRNAs, wherein the majority of representative shRNAs are associated with a barcode tag. Optionally, the representative shRNAs are partially complementary to representative genes, and wherein a majority of representative gene are known or suspected to be involved in a cancer.

In certain aspects, the invention provides methods of determining a function of a gene comprising: introducing small hairpin RNA which targets mRNA of the gene into cells; maintaining the cells under conditions in which the small hairpin RNA is stably expressed and RNA interference of the mRNA occurs; introducing the cells into a non-human mammal, thereby producing a knockout non-human mammal; and assessing the phenotype of the knock-out non-human mammal compared to a control mammal, thereby identifying a function of the gene. In some embodiments, a the invention provides a method of determining the contribution of a gene to a condition comprising: a) introducing small hairpin RNA which vary in their ability to inactivate mRNA of the gene into cells, thereby producing a panel of a discrete set of cells in which the mRNA of the gene is inactivated to varying degrees in each set of cells; b) maintaining the cells under conditions in which the small hairpin RNA is stably expressed and RNA interference of the mRNA occurs; c) introducing each set of cells into a separate non-human mammal, thereby producing a panel of knockout non-human mammals in which the mRNA of the gene is inactivated to varying degrees in each non-human mammal; and d) assessing the phenotype of each knock-out non-human mammal compared to a control mammal, thereby determining the contribution of the gene to the condition.

In certain aspects the invention provides a method of engineering cells ex vivo so that the cells exhibit reduced expression of a gene product comprising: a) removing cells from a host; and b) introducing a construct encoding a small hairpin RNA into the cells such that the small RNA is stably expressed and induces RNA interference of the gene product.

In certain aspects the invention relates to the discovery that a cell expressing a shRNA construct may retain a stable RNA interference effect even after excision or other inactivation of the shRNA construct. In certain embodiments, the invention provides a method for introducing into a subject a population of stem cells having partial or complete loss of function of a target gene, the method comprising: a) introducing a nucleic acid construct encoding an shRNA into stem cells to generate transfected stem cells, wherein the shRNA is complementary to a portion of the target gene, such that expression of the target gene is decreased; b) removing or inactivating the nucleic acid construct; c) verifying that expression of the target gene remains decreased; d) introducing the stem cells into a subject, wherein the stem cells propagate within the subject and retain partial to complete loss of function of the target gene. Optionally, the nucleic acid construct comprises a lox site and removing or inactivating the nucleic acid construct comprises introducing or activating Cre.

Another aspect of the invention relates to a mammalian cell comprising a stably-integrated polynucleotide encoding an RNAi construct, wherein the expression of the RNAi construct is conditional.

In certain embodiments, the mammalian cell is a stem cell, e.g., an embryonic stem cell, or a hematopoietic stem cell.

In certain embodiments, the mammalian cell is a human or mouse cell.

In certain embodiments, the mammalian cell is in culture, or in a non-human animal.

In certain embodiments, the polynucleotide encoding the RNAi construct is a plasmid.

In certain embodiments, the polynucleotide encoding the RNAi construct is a retroviral vector or a lentiviral vector.

In certain embodiments, the polynucleotide encoding the RNAi construct is stably integrated into a defined locus of the genome.

In certain embodiments, a single copy of the polynucleotide encoding the RNAi construct is stably integrated into a defined locus of the genome.

In certain embodiments, the polynucleotide encoding the RNAi construct is stably integrated into a defined locus of the genome via Cre-mediated recombination.

In certain embodiments, the polynucleotide encoding the RNAi construct is stably integrated into a defined locus of the genome via FLP/FRT-mediated recombination.

In certain embodiments, the defined locus is ColA1.

In certain embodiments, the RNAi construct is short hairpin RNA (shRNA) or microRNA (miRNA).

In certain embodiments, the miRNA is mir30-based.

In certain embodiments, the conditional expression of the RNAi construct is temporal, tissue-specific, inducible, and/or reversible.

In certain embodiments, the conditional expression of the RNAi construct is reversed by excision of the stably integrated polynucleotide encoding the RNAi construct.

In certain embodiments, the expression of the RNAi construct is conditional on the presence or absence of a substance administered to the mammalian cell.

In certain embodiments, the substance is tetracyclin or an analog thereof.

In certain embodiments, the conditional expression of the RNAi construct is reversed by adding or withdrawing the substance.

In certain embodiments, the polynucleotide encoding the RNAi construct comprises an inducible tetracyclin-responsive promoter or TRE.

In certain embodiments, the mammalian cell further comprises a coding sequence for a tet-transactivator protein (tTA) or a reverse tet-transactivator protein (rtTA).

In certain embodiments, the tTA or rtTA exhibits tissue-specific expression.

In certain embodiments, the polynucleotide encoding the RNAi construct comprises a coding sequence for a fluorescent protein, a drug resistant gene, a SIN LTR, and/or an Internal Ribosomal Entry Site (IRES).

In certain embodiments, the fluorescent protein is GFP, or variants/mutants thereof.

In certain embodiments, the expression of the RNAi construct is coupled to the expression of the fluorescent protein.

In certain embodiments, the RNAi construct is complementary to a portion of a target gene in the mammalian cell, and, when expressed, the RNAi construct causes partial or complete loss of function of the target gene.

Another aspect of the invention relates to a non-human mammal derived from or produced by the subject embryonic stem cell (e.g., those with conditional RNAi constructs stably integrated in the genome, preferably at predetermined locus, such as ColA1).

In certain embodiments, the mammal is produced by blastocyst injection, or by tetraploid embryo complementation using the subject embryonic stem cell.

A related aspect of the invention relates to a non-human mammal comprising the subject mammalian cell, or a further proliferation or differention progeny thereof.

In certain embodiments, the RNAi construct is complementary to a portion of a target gene in the non-human mammal, and wherein the target gene participates in a disease process in the non-human mammal.

In certain embodiments, the target gene is a tumor suppressor gene.

In certain embodiments, the mammalian cell is an autologous cell derived from the non-human mammal.

In certain embodiments, the non-human mammal is a mouse.

Another aspect of the invention provides a method for identifying a gene that affects the sensitivity of tumor cells to a chemotherapeutic agent, the method comprising: (a) introducing into a subject a transfected stem cell comprising a stably-integrated polynucleotide encoding an RNAi construct, wherein the RNAi construct is complementary to at least a portion of a target gene, wherein the expression of the RNAi construct is conditional, and, wherein the transfected stem cell exhibits decreased expression of the target gene upon expression of the RNAi construct, and gives rise to a transfected tumor cell in vivo; (b) evaluating the effect of the chemotherapeutic agent on the transfected tumor cell.

In certain embodiments, evaluating the effect of the chemotherapeutic agent on the transfected tumor cell comprises: administering the chemotherapeutic agent to the subject and measuring the quantity of tumor cells derived from the transfected stem cell.

In certain embodiments, the method further comprises comparing the quantity of tumor cells derived from the transfected stem cell to the quantity of tumor cells derived from the transfected stem cell in a control subject that has not received the chemotherapeutic agent.

Another aspect of the invention provides a method of determining a function of a gene in a cell, comprising: (a) introducing into the cell a stably-integrated polynucleotide encoding an RNAi construct which targets mRNA of the gene, wherein the expression of the RNAi construct is conditional; (b) maintaining the cell under conditions in which the RNAi construct is expressed and RNA interference of the mRNA occurs; (c) assessing the phenotype of the cell, or the phenotype of a non-human mammal comprising the cell, as compared to a control; thereby identifying a function of the gene.

In certain embodiments, the control is an identical cell maintained under conditions in which the RNAi construct is not expressed, or an identical non-human mammal comprising the cell maintained under conditions in which the RNAi construct is not expressed.

In certain embodiments, the method further comprises: (d) maintaining the cell under conditions in which the RNAi construct is not expressed and RNA interference of the mRNA does not occur; (e) assessing the phenotype of the cell, or the phenotype of a non-human mammal comprising the cell.

In certain embodiments, the non-human mammal is a mouse.

A further aspect of the invention provides a polynucleotide encoding an RNAi construct, wherein the expression of the RNAi construct is conditional.

It is contemplated that all embodiments of the invention described herein, including those that might be more relevant to one or more aspects of the invention than others, are generally applicable to all aspects of the invention, unless specifically indicated otherwise or clearly inapplicable.

### BRIEF DESCRIPTION OF THE DRAWINGS

FIG. 1 is a schematic diagram showing the process of generation of shRNA expressing lymphomas.

FIG. 2 is a schematic diagram showing the retroviral construct design for p53-A, p53-B and p53-C. p53-A has an MMLV retroviral backbone, while p53-B and p53-C are derived from MSCV.

FIG. 3 is a diagram showing the approximate location of the hairpin sequence on the p53 cDNA.

FIG. 4 is a diagram showing the PCR amplification of tumor and control DNA with shRNA-specific primers. Both tumors show the presence of the hairpin construct, while control pre-infection stem cells do not.

FIG. 5 is a diagram showing survival curves for mice injected with stem cells infected with either Control or p53 shRNA constructs.

FIGS. 6A-6C are H&E slides of a lymphoma (FIG. 6A), a lung (FIG. 6B) and a spleen (FIG. 6C) from a mouse with shp53-induced tumors. Lymphoma pathology and aggressive lung and spleen metastasis resemble that seen in p53−/−tumors.

FIG. 6D is a TUNEL staining showing only low levels of apoptosis in shp53-induced lymphomas, a characteristic of p53−/− tumors.

FIG. 7 is a Western analysis for p53 levels in Murine Embryo Fibroblasts (MEFs) infected with various hairpins targeting p53. Cells were treated with 0.5 ug/ml adriamycin for 6 hours to induce p53 levels. All p53 shRNAs show a reduction in p53 induction, while a GFP shRNA had no effect on p53 levels. Tubulin controls were provided to confirm equal amounts of total protein in each lane.

FIG. 8 is a PCR reaction, designed to amplify both the WT and KO p53 allele, and shows the maintenance of the WT allele in a tumor expressing a p53 shRNA. An MSCV control shows loss of the WT allele, while a bcl-2 control shows retention of the WT allele.

FIG. 9: Heritable repression of Neil1 expression by RNAi in several tissues. (a) Expression of Neil1 mRNA in the livers of three mice containing the Neil1 shRNA transgene (shRNA-positive) or three siblings lacking the transgene (shRNA-negative) was assayed by RT-PCR (top row is Neil1). An RT-PCR of β-actin was done to ensure that equal quantities of mRNAs were tested for each mouse (second row). Expression of the neomycin resistance gene (neo), carried on the shRNA vector, was tested similarly (third row). Finally, the mice were genotyped using genomic DNA that was PCR-amplified with vector-specific primers (bottom row). (b) Similar studies were performed in the heart. (c) Similar studies were performed in the spleen. Animal procedures have been approved by the SUNY, Stony Brook Institutional Animal Care and Use Committee (IACUC).

FIG. 10: Reduction in Neil1 protein correlates with the presence of siRNAs. (a) Expression of Neil1 protein was examined in protein extracts from the livers of mice carrying the shRNA transgene (shRNA-positive) or siblings lacking the transgene (shRNA-negative) by western blotting with Neil1-specific antiserum. A western blot for PCNA was used to standardize loading. (b) The presence of siRNAs in RNA derived from the livers of transgenic mice as assayed by northern blotting using a 300 nt probe, part of which was complementary to the shRNA sequence. Applicants note siRNAs only in mice transgenic for the shRNA expression cassette.

FIG. 11. A. Graph showing a shorter lymphoma onset time Bim or Puma shRNA mice. B, C. Bim and Puma expression are decreased in tumor cells by targeted shRNA.

FIG. 12. Survival of tumor cells carrying Bim shRNA as compared to control tumors, during treatment with adriamycin.

FIG. 13. Diagram of shRNA screening assay to identify tumor sensitizing shRNAs.

FIG. 14. FACS analysis of GFP containing cells in pre-treatment and relapsed tumors.

FIG. 15. A. A diagram of a Self-Inactivating retroviral vector (SIN vector) for use with shRNA. B. Demonstration of effectiveness of SIN vector and standard vector in RNA interference.

FIG. 16. Southern blot analysis of proviral transgene insertions in the p53C shRNA founder mice. Transgenic founders #3, #8, and #10 have a single proviral insertions site, while the rest of the mice were non-transgenic.

FIG. 17. Western analysis of p53 in dermal fibroblasts of p53C shRNA lentiviral transgenic mice (#'s 3, 8, and 10) and non-transgenic littermate controls (#'s 1 and 2), treated with 0.5 ug adriamycin per ml for approximately 6 hours. Lanes 1 and 2 are MEFs infected with either MSCV or p53C shRNA and treated with adriamycin.

FIG. 18. Colony formation assay using dermal fibroblasts cultured from lentiviral-mediated p53C shRNA transgenic mice and non-transgenic littermate control. Cells were plated at the indicated cell numbers, and allowed to grow for approximately 3 weeks.

FIG. 19. A. Schematic representation of the screening process using population approaches in which biological stimuli are applied to populations of cells containing barcoded shRNAs. B. Images of arrays in the Cy3 and Cy5 channels of a self-self library hybridization. C. A log-log plot of intensities in Cy3 and Cy5 channels.

FIG. 20. A diagram of a methodology for identifying genes that participate in chemotherapeutic resistance and sensitivity.

FIG. 21. Cells were infected with RCAS shp53C or a control vector, selected with puromycin for 3 days, and subsequently plated at 25,000 cells per well. Cells were treated with 0.5 ug/ml adriamycin to induce p53.

FIG. 22. Cells were infected with either RCAS shp53C or control vector, selected with puromycin for 3 days, and subsequently plated at the indicated cell numbers per well and allowed to grow for approximately 2 wks. Data reveal enhanced cell growth for cells expressing RCAS shp53C.

FIG. 23. Diagram of site specific shRNA insertion system.

FIG. 24. Suppression of luc activity in cells expressing luc shRNAs. Luciferace activity in the shRNA expressing cells is shown relative to cells not expressing shRNA.

FIG. 25. A. Excisable shRNA expression vector harboring tamoxifen-regulated cre. B. Wild type MEFS were infected with the Cre-loxP-U6p53CshRNA-PIG virus, and these cells show stable suppression of p53 expression by Western blot.

FIG. 26. Addition of 0.5 μM 4-hydroxytamoxifen (4OHT) to cultured cells infected with MSCV CreER/loxP U6p53C PIG virus results in deletion of the provirus from the genome, as measured by Southern blot using a probe that hybridizes to the GFP cassette in the provirus (A). As expected, 4OHT treatment and excision of the provirus also leads to loss of GFP expression, as measured by Western blot (B) or FACS(C).

FIG. 27. MSCV Cre/loxP U6p53C PIG in cultured mouse embryonic fibroblasts. Control cells are in the upper panels. Lower panels are tamoxifen treatment panels.

FIG. 28. A diagram of a second generation vector.

FIG. 29. Western blot showing p53 protein levels in cultured murine embryonic fibroblast cells infected with MSCV Cre/loxP U6p53C PIG or a control vector (MSCV PIG). Virally infected, puromycin selected cells were cultured for 6 days, treated with 0.5 uM OHT or vehicle for 24 h, then cultured for a further 6 days. Immediately before harvesting, cells were treated as indicated for 4 h with 0.5 ug/mL adriamycin (ADR), a DNA damaging agent that causes massive induction of p53 in control (MSCV PIG) infected cells. Minimal p53 induction is observed in MSCV Cre/loxP U6p53C PIG infected cells, even 6 days after OHT treatment.

FIG. 30. Improved vector design and functional in vitro assays. (a) Schematic design of TGM and TMP vectors. (b, c) Western-blot analyses of sh-p53.1224 and sh-p16/19.478 in the TGM vector. (b) MEFs were infected with TGM, TMG, or TMP vectors+/−shp53.1224 and selected in puromycin for 4 days. TMG is an intermediary vector (schematic design not shown). Following selection, cells were treated with adriamycin for 5 hrs prior to harvest to induce p53 expression. (c) MEFs were infected with TGM-p16/19.478 and selected in puromycin for 4 days. MEFs were treated for 0, 2, 4 or 8 days with doxycycline to shutdown expression of sh-p16/19.478. “pA” stands for polyadenylation signal.

FIG. 31: Germline inheritance of a regulatable TRE-p53.1224 transgene (above). (a) Schematic design of TREp53.1224 fragment used for pronuclear injection. (b, c, d) Western-blot analyses for Trp53 expression. (b) MEFs were isolated from founder A and founder B F1 embryos and infected with tTA protein. Genotyping for TRE-p53.1224 transgene was performed by PCR. (c) MEFs of founder A F1 embryos infected with tTA (right 4 lanes) or uninfected (left 4 lanes). (d) Doxycycline (Dox) temporal-response analysis of Trp53 expression in MEFs from founder A.

FIG. 32: (a) Schematic design of TRE-GFP-miR30 fragments, TGM and TGMpA, used for pronuclear injection. (b) TGMpA-p53.1224; CMV-rtTA double transgenic mice and single transgenic littermates (left). Only double transgenic mice display GFP expression (right).

FIG. 33: (a) Western-blot analyses to compare shRNAs targeting p16 and p19. MEFs were infected with MGPP-p16/19 constructs at low MOI (8% by GFP) and selected in puromycin for 4 days. Following selection, cells were grown for an addition 10 days and whole populations were harvested. (b) Colony formation assay comparing TGM-p16/19.478 and LMP-p16/19.478 (LTR-miR30-PGK-Puro) infected MEFs. Cells were plated at low density (10,000 cells per 10 cm dish). Colony formation is consistent with the known effects of p19ARF deficiency in MEFs.

FIG. 34: (a) Schematic design of the ColA1 locus (chromosome 11) in KH2 cells. ColA1 was previously targeted with an frt-hygro-pA “homing cassette downstream of the Type I collagen gene, rendering Neo-resistance and Hygro-sensitivity. Electroporation of pBS31-TGM and pCAGGS FLPe induces Frt recombination, eliminating PGK-NeoR and incorporating pBS31-TGM to render HygroR. (b) Southern-blot analysis of genomic DNA isolated from selected GFP positive clones and digested with SpeI demonstrates correct targeting.

FIG. 35: (a) Western blot analyses of select ColA1-TGM-p53.1224 targeted clones. ES cells were treated with doxycycline for 4 days and adriamycin for 2 hours prior to harvesting. (b) Western blot analyses of clone 4B1 ES cells treated with doxycycline for 0, 2, 4 or 8 days. Following 8 days of treatment, cells were removed from doxycycline for 2, 4 or 8 days. All cells were treated with adriamycin for 2 hours prior to harvest. (c) Two-week old ColA1-TGM-p16/19.478 ES cell derived mice treated with or without doxycycline for 4 days. GFP expression is visible in the skin after doxycycline treatment. (d) Western blot analyses of whole tissues isolated from a ColA1-TGM-p16/19.478 ES cell derived mouse treated with doxycycline for >4 days. [1—adipose; 2—bladder; 3—bone marrow; 4—brain; 5—colon; 6—esophagus; 7—heart; 8—intestine; 9—kidney; 10—liver; 11—lung; 12—muscle; 13—pancreas; 14—salivary gland; 15—skin; 16—spleen; 17—stomach; 18—testis; 19—thymus].

FIG. 36: (a) Western blot analyses of primary MEFs harvested from a cross between wild-type C57BL/6 and ColA1-TGMp16/19.478 mice. All MEFs were treated with doxycycline for 0, 2, 4 or 8 days prior to harvesting. (b) Colony formation assay with primary MEFs harvested from a cross between CMV-rtTA and ColA1-TGMp16/19.478 mice [UT—untreated]).

FIG. 37. (a) Schematic representation of the ColA1 locus (chromosome 11) and strategy for FLPe-mediated recombination of the TGM-FLP-In vector in HK2 ES cells. (b) Strategy for generating targeted colA1-TGM ES cells. (c) Time line comparing recombination strategies for generating knock-out vs. RNAi mice.

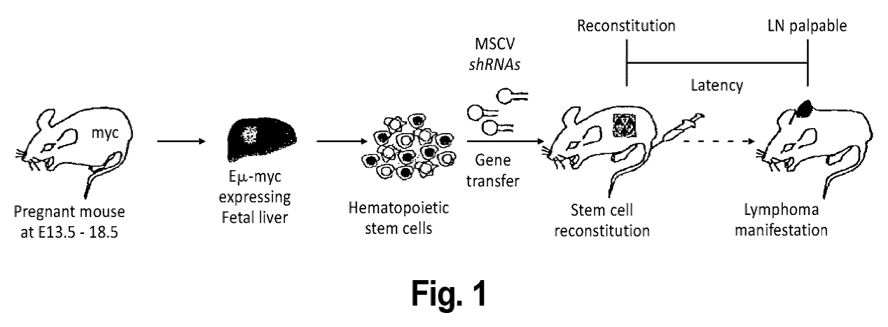

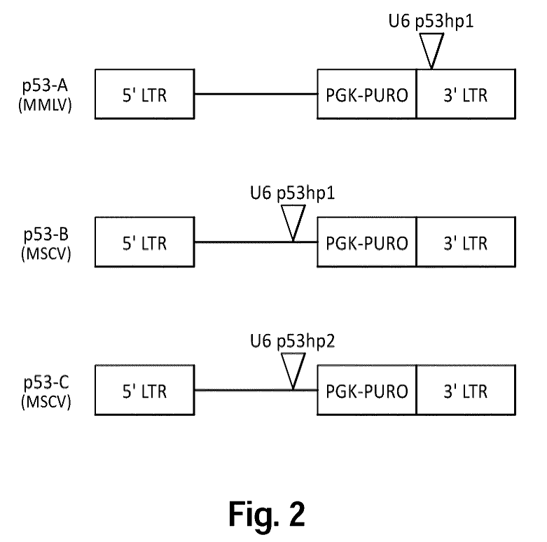

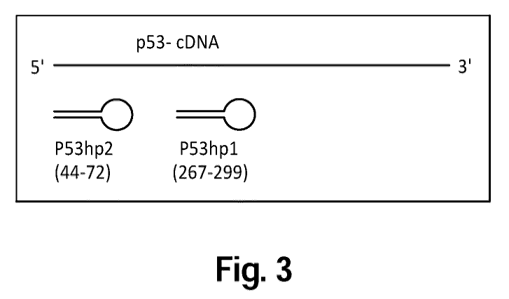

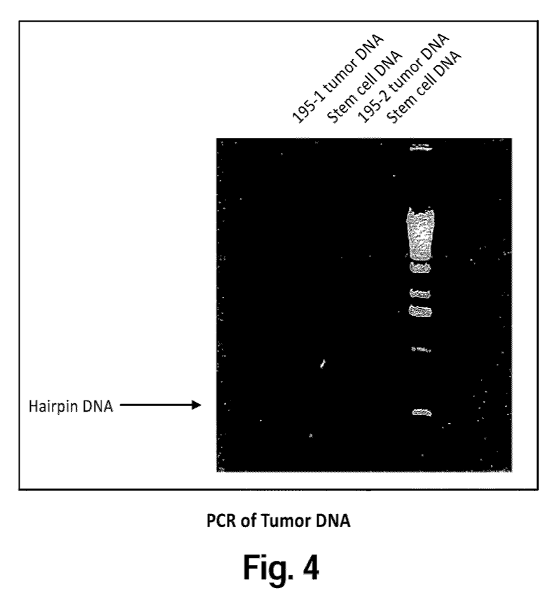

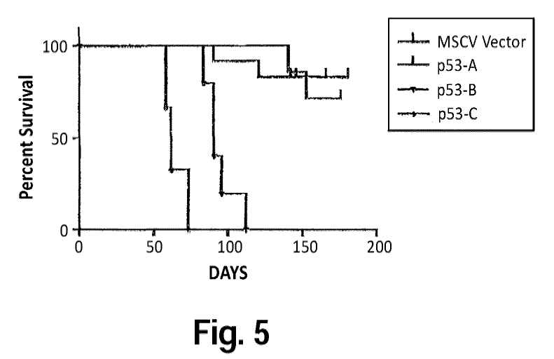

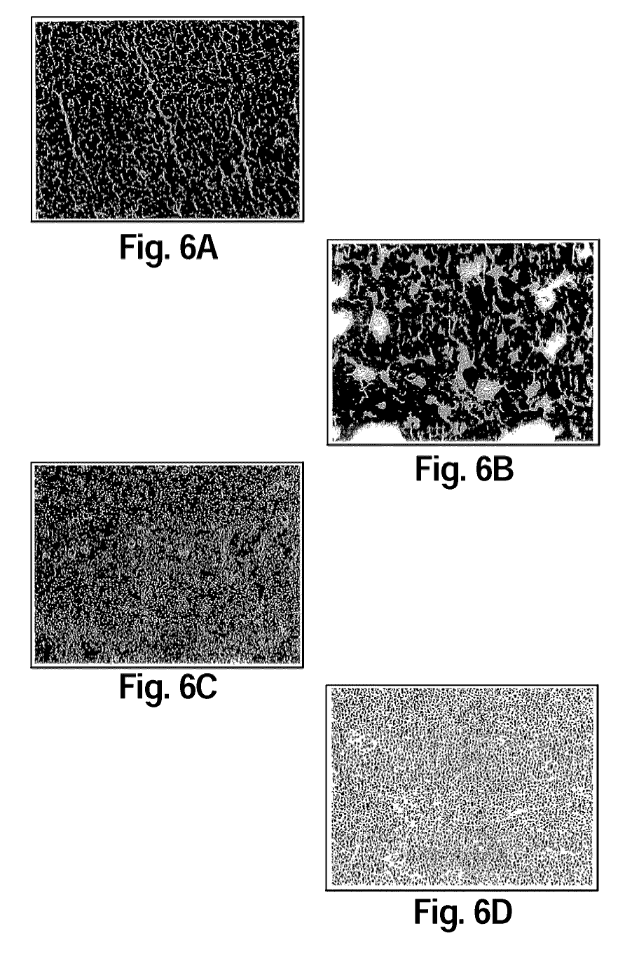

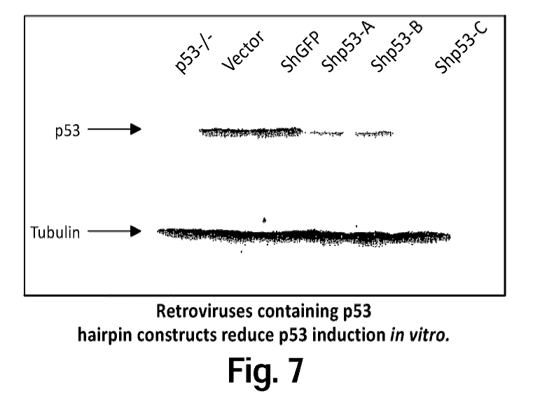

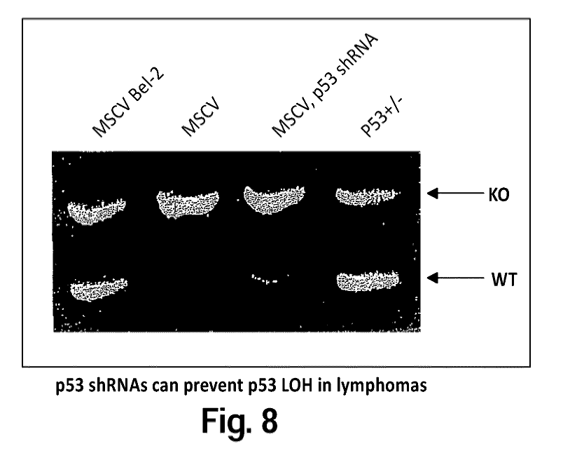

### DETAILED DESCRIPTION OF THE INVENTION

### 1. Overview

In certain aspects, the invention provides systems which use RNA interference to stably, conditionally (e.g., with spacial, temporal, and/or reversible control) and specifically target and decrease the expression of one or more target genes in cells. Recent work has shown that the RNA interference effects of exogenously provided dsRNAs can be recapitulated in mammalian cells by the expression of single RNA molecules which fold into stable “hairpin” structures (Paddison et al. Genes Dev 16(8):948-58 (2002)). Transient transfection of plasmids encoding small “hairpin” RNAs (shRNAs) can achieve a near complete reduction in the levels of a specific protein in a cell. Applicants have now demonstrated that shRNAs can be stably introduced into mammalian cells, preferably in a site-specific manner, introduced into a living organism and propagated without significant loss of the RNA interference effect. Furthermore, the stably integrated RNAi constructs may be conditionally expressed (e.g., expression may be turned on or off in a tissue-specific or reversible manner). A variety of experiments substantiating the discovery are presented in detail in the Examples below.

To summarize, in one such experiment, shRNAs targeted to p53 were introduced into mouse stem cells in culture and transplanted into mice. Applicants have detected the presence of shRNAs in transplanted cells over three months after transplantation. Cells manipulated according to the disclosed methodology may be introduced into a mammal (or used to generate a mammal) and propagated in vivo without significant loss of the RNA interference effects in the cells or their progeny. In certain embodiments, the system takes advantage of gene transfer of DNA or RNA constructs encoding short hairpin RNAs into cells.

Accordingly, in certain aspects, the invention provides systems, cells, or non-human animal model for reducing the expression of genes (e.g., “knock-out” or partial reduction) in an in vivo model and analyzing the results in a rapid manner. This technology potentially bypasses both the developmental issues of embryonic lethality and compensation seen in traditional “knock-out” mouse systems. RNA inhibition has previously been used to suppress gene expression in mammalian cells in vitro. These groups have also transplanted these cultured cells as xenografts into nude mice. However, the experiments described in this document are the first to stably express shRNAs in stem cells and subsequently use those stem cells to reconstitute a fully functional organ with a targeted gene “knock-out.”

Applicants have further discovered a wide range of technological and therapeutic applications for implantable stem cells transfected with stable RNAi constructs.

In certain aspects, methods disclosed herein may be used for ex vivo stem cell therapies. For example, an autologous or heterologous stem cell population may be transfected with a stable RNA interference construct and introduced into a patient, where the modified cells perform a therapeutic function. It is important to note that RNA interference may be used to cause both decreased (e.g., direct RNA interference) or increased expression of genes (e.g., indirect effect). For example, although RNA interference will decrease the expression of a target gene, the target gene itself may be a negative regulator, and therefore the RNA interference will indirectly cause increased expression of the negative regulator.

In further aspects, methods disclosed herein may be used to assess the positive or negative effects of a RNAi on an in vivo process. For example, as described in the examples below, stem cells transfected with a stable shRNA construct may be used to identify gene that contribute to chemotherapeutic sensitivity or resistance in tumor cells. In certain embodiments, such screening methods may be performed in a high throughput format.

The subject systems, cells and non-human animal models can be used to rapidly and efficiently generate models (e.g., mice) that contain a conditional RNAi construct, such that nearly any gene in the host genome can be spatially, temporarily, and reversibly regulated by, for example, crossing these targeted mice to tissue-specific tTA or rtTA mouse lines. Such systems, cells and non-human animal models can serve as powerful tools to dissect not only the impact of knockdown of specific gene targets, but also the effect of gene re-establishment in gene loss-of-function disease models. For example, in cancer, RNAi can be induced or shut down to conditionally turn off or turn on the expression of certain genes, thus mimicking the effect of deliverying small molecule therapies, and allowing the assessment of the impact of specific gene knockdown globally in a disease setting.

In certain embodiments, when conditional expression of the RNAi construct is coupled to the expression/activity of certain markers, such as a visible GFP marker, the system also allows monitoring of RNAi production in live animals without having to sacrifice the animals, thus live disease progression and the time course of host response to therapy can be monitored in real time.

More details of the invention are further described in the sections below.

### 2. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article, unless context clearly indicates otherwise. By way of example, “an element” means one element or more than one element.

The term “adult stem cell” is used herein to refer to a stem cell obtained from any non-embryonic tissue. For example, cells derived from fetal tissue and amniotic or placental tissue are included in the term adult stem cell. Cells of these types tend to have properties more similar to cells derived from adult animals than to cells derived from embryonic tissue, and accordingly, for the purposes of this application stem cells may be sorted into two categories: “embryonic” and “adult” (or, equivalently, “non-embryonic”).

The term “culturing” includes exposing cells to any condition. While “culturing” cells is often intended to promote growth of one or more cells, “culturing” cells need not promote or result in any cell growth, and the condition may even be lethal to a substantial portion of the cells.

A later cell is “derived” from an earlier cell if the later cell is descended from the earlier cell through one or more cell divisions. Where a cell culture is initiated with one or more initial cells, it may be inferred that cells growing up in the culture, even after one or more changes in culture condition, are derived from the initial cells. A later cell may still be considered derived from an earlier cell even if there has been an intervening genetic manipulation.

The term “including” is used herein to mean, and is used interchangeably with, the phrase “including but not limited to”.

The term “or” is used herein to mean, and is used interchangeably with, the term “and/or”, unless context clearly indicates otherwise.

A “patient” or “subject” to be treated by the method of the invention can mean either a human or non-human animal, preferably a mammal.

The term “percent identical” refers to sequence identity between two amino acid sequences or between two nucleotide sequences. Percent identity can be determined by comparing a position in each sequence which may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

“Stem cell” describes cells which are able to regenerate themselves and also to give rise to progenitor cells which ultimately will generate cells developmentally restricted to specific lineages.

### 3. Hairpin RNAi Constructs, Vectors and Cells

Many embodiments of the invention employ single-stranded RNA molecules containing an inverted repeat region that causes the RNA to self-hybridize, forming a hairpin structure. shRNA molecules of this type may be encoded in RNA or DNA vectors. The term “encoded” is used to indicate that the vector, when acted upon by an appropriate enzyme, such as an RNA polymerase, will give rise to the desired shRNA molecules (although additional processing enzymes may also be involved in producing the encoded shRNA molecules). As described herein, vectors comprising one or more encoded shRNAs may be transfected into cells ex vivo, and the cells may be introduced into mammals. The expression of shRNAs may be constitutive or regulated in a desired manner. Other technologies for achieving RNA interference in vivo were unreliable; certain constructs were expressible in stem cells but not in differentiated cells, or vice versa. Technology described herein makes it possible to achieve either constitutive or highly regulated expression of shRNAs in vivo across the spectrum of cell types, thereby permitting tightly controlled regulation of target genes in vivo.

A double-stranded structure of an shRNA is formed by a single self-complementary RNA strand. RNA duplex formation may be initiated either inside or outside the cell. Inhibition is sequence-specific in that nucleotide sequences corresponding to the duplex region of the RNA are targeted for genetic inhibition. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Because 100% sequence identity between the RNA and the target gene is not required to practice the present invention, the invention has the advantage of being able to tolerate sequence variations that might be expected due to genetic mutation, strain polymorphism, or evolutionary divergence. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

An endogenous RNA polymerase of the cell may mediate transcription of an shRNA encoded in a nucleic acid construct. The shRNA construct may also be synthesized by a bacteriophage RNA polymerase (e.g., T3, T7, SP6) that is expressed in the cell. In preferred embodiments, expression of an shRNA is regulated by an RNA polymerase III promoters; such promoters are known to produce efficient silencing. While essentially any PolII promoters may be used, desirable examples include the human U6 snRNA promoter, the mouse U6 snRNA promoter, the human and mouse H1 RNA promoter and the human tRNA-val promoter. A U6 snRNA leader sequence may be appended to the primary transcript; such leader sequences tend to increase the efficiency of sub-optimal shRNAs while generally having little or no effect on efficient shRNAs. For transcription from a transgene in vivo, a regulatory region (e.g., promoter, enhancer, silencer, splice donor and acceptor, polyadenylation) may be used to regulate expression of the shRNA strand (or strands). Inhibition may be controlled by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. The use and production of an expression construct are known in the art (see also WO 97/32016; U.S. Pat. Nos. 5,593,874, 5,698,425, 5,712,135, 5,789,214, and 5,804,693; and the references cited therein).

In a preferred embodiment, a shRNA construct is designed with 29 bp helices following a U6 snRNA leader sequence with the transcript being produced by the human U6 snRNA promoter. This transcription unit may be delivered via a Murine Stem Cell Virus (MSCV)-based retrovirus, with the expression cassette inserted downstream of the packaging signal. Further information on the optimization of shRNA constructs may be found, for example, in the following references: Paddison, P. J., A. A. Caudy, and G. J. Hannon, Stable suppression of gene expression by RNAi in mammalian cells. Proc Natl Acad Sci USA, 2002. 99(3): p. 1443-8; 13.Brummelkamp, T. R., R. Bernards, and R. Agami, A System for Stable Expression of Short Interfering RNAs in Mammalian Cells. Science, 2002. 21: p. 21; Kawasaki, H. and K. Taira, Short hairpin type of dsRNAs that are controlled by tRNA(Val) promoter significantly induce RNAi-mediated gene silencing in the cytoplasm of human cells. Nucleic Acids Res, 2003. 31(2): p. 700-7; Lee, N. S., et al., Expression of small interfering RNAs targeted against HIV-1 rev transcripts in human cells. Nat Biotechnol, 2002. 20(5): p. 500-5; Miyagishi, M. and K. Taira, U6 promoter-driven siRNAs with four uridine 3′ overhangs efficiently suppress targeted gene expression in mammalian cells. Nat Biotechnol, 2002. 20(5): p. 497-500; Paul, C. P., et al., Effective expression of small interfering RNA in human cells. Nat Biotechnol, 2002. 20(5): p. 505-8.

An shRNA will generally be designed to have partial or complete complementarity with one or more target genes (i.e., complementarity with one or more transcripts of one or more target genes). The target gene may be a gene derived from the cell, an endogenous gene, a transgene, or a gene of a pathogen which is present in the cell after infection thereof. Depending on the particular target gene, the nature of the shRNA and the level of expression of shRNA (e.g. depending on copy number, promoter strength) the procedure may provide partial or complete loss of function for the target gene. Quantitation of gene expression in a cell may show similar amounts of inhibition at the level of accumulation of target mRNA or translation of target protein.

“Inhibition of gene expression” refers to the absence or observable decrease in the level of protein and/or mRNA product from a target gene. “Specificity” refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

As disclosed herein, the present invention is not limited to any type of target gene or nucleotide sequence. The following classes of possible target genes are listed for illustrative purposes: developmental genes (e.g., adhesion molecules, cyclin kinase inhibitors, Writ family members, Pax family members, Winged helix family members, Hox family members, cytokines/lymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, EBRB2, ETSI, ETS1, ETV6, FGR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES); tumor suppressor genes (e.g., APC, BRCA1, BRCA2, MADH4, MCC, NF1, NF2, RB1, p53, BIM, PUMA and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases).

Promoters/enhancers which may be used to control the expression of a shRNA construct in vivo include, but are not limited to, the PolIII human or murine U6 and H1 systems, the cytomegalovirus (CMV) promoter/enhancer, the human β-actin promoter, the glucocorticoid-inducible promoter present in the mouse mammary tumor virus long terminal repeat (MMTV LTR), the long terminal repeat sequences of Moloney murine leukemia virus (MuLV LTR), the SV40 early or late region promoter, the promoter contained in the 3′ long terminal repeat of Rous sarcoma virus (RSV), the herpes simplex virus (HSV) thymidine kinase promoter/enhancer, and the herpes simplex virus LAT promoter. Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., an immunoglobulin promoter, and from heat-shock promoters, provided such promoters are compatible with the host cell systems. Inducible systems, such as Tet promoters may be employed. In addition, recombinase systems, such as Cre/lox may be used to allow excision of shRNA constructs at desired times. The Cre may be responsive (transcriptionally or post-transcriptionally) to an external signal, such as tamoxifen.

In certain embodiments, a vector system for introducing shRNA constructs into cells are retroviral vector systems, such as lentiviral vector systems. Lentiviral systems permit the delivery and expression of shRNA constructs to both dividing and non-dividing cell populations in vitro and in vivo. Examples of Lentiviral vectors are those based on HIV, FIV and EIAV. See, e.g., Lois, C., et al., Germline transmission and tissue-specific expression of transgenes delivered by lentiviral vectors. Science, 2002. 295(5556): p. 868-72. Most viral systems contain cis-acting elements necessary for packaging, while trans-acting factors are supplied by a separate plasmid that is co-transfected with the vector into a packaging cell line. In certain embodiments, a highly transfectable 293 cell line may be used for packaging vectors, and viruses may be pseudotyped with a VSV-G envelope glycoprotein for enhanced stability and to provide broad host range for infection. In certain aspects, the invention provides novel vectors adapted for use with shRNA expression cassettes. For example, a Gateway recipient sequence may be inserted downstream of the packaging signal to facilitate movement of the shRNA construct to and from different vector backbones by simple recombination. As another example, recombination signals may be inserted to facilitate in vivo transfer of shRNAs from, e.g., a genome-wide shRNA library.

The type of vector and promoters to be employed should be selected, in part, depending on the organism and cell type to be affected. In the case of ex vivo stem cell therapy for human patients, a vector and promoter that are capable of transfection and expression in human cells should be selected.

In certain embodiments, retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector may be employed to transduce packaging cell lines to form producer cell lines. Examples of packaging cells which may be transfected include, but are not limited to, the PE501, PA317, R-2, R-AM, PA12, T19-14.times., VT-19-17-H2, RCRE, RCRIP, GP+E-86, GP+envAml2, and DAN cell lines as described in Miller, Human Gene Therapy 1:5-14 (1990), which is incorporated herein by reference in its entirety. The vector may transduce the packaging cells through any means known in the art. A producer cell line generates infectious retroviral vector particles which include polynucleotide encoding a polypeptide of the present invention. Such retroviral vector particles then may be employed, to transduce eukaryotic cells, either in vitro or in vivo. The transduced eukaryotic cells will express a polypeptide of the present invention.

In certain embodiments, cells are engineered using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377. For example, an AAV vector may include all the sequences necessary for DNA replication, encapsidation, and host-cell integration. The recombinant AAV vector may be transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells.

Essentially any method for introducing a nucleic acid construct into cells may be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle may be used to accomplish both efficient introduction of an expression construct into the cell and transcription of the encoded shRNA. Other methods known in the art for introducing nucleic acids to cells may be used, such as lipid-mediated carrier transport, chemical mediated transport, such as calcium phosphate, and the like. Thus the shRNA-encoding nucleic acid construct may be introduced along with components that perform one or more of the following activities: enhance RNA uptake by the cell, promote annealing of the duplex strands, stabilize the annealed strands, or otherwise increase inhibition of the target gene.

Cells to be transfected may be essentially any type of cell for implantation into in a subject. The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands. After transfection, stem cells may be administered as stem cells to a subject, or cultured to form further differentiated stem cells (e.g., embryonic stem cells cultured to form neural, hematopoietic or pancreatic stem cells) or cultured to form differentiated cells.

Stem cells may be stem cells recently obtained from a donor, and in certain preferred embodiments, the stem cells are autologous stem cells. Stem cells may also be from an established stem cell line that is propagated in vitro. Suitable stem cells include embryonic stems and adult stem cells, whether totipotent, pluripotent, multipotent or of lesser developmental capacity. Stem cells are preferably derived from mammals, such as rodents (e.g. mouse or rat), primates (e.g. monkeys, chimpanzees or humans), pigs, and ruminants (e.g. cows, sheep and goats). Examples of mouse embryonic stem cells include: the JM1 ES cell line described in M. Qiu et al., Genes Dev 9, 2523 (1995), and the ROSA line described in G. Friedrich, P. Soriano, Genes Dev 5, 1513 (1991), and mouse ES cells described in U.S. Pat. No. 6,190,910. Many other mouse ES lines are available from Jackson Laboratories (Bar Harbor, Me.). Examples of human embryonic stem cells include those available through the following suppliers: Arcos Bioscience, Inc., Foster City, Calif., CyThera, Inc., San Diego, Calif., BresaGen, Inc., Athens, Ga., ES Cell International, Melbourne, Australia, Geron Corporation, Menlo Park, Calif., Göteborg University, Göteborg, Sweden, Karolinska Institute, Stockholm, Sweden, Maria Biotech Co. Ltd.—Maria Infertility Hospital Medical Institute, Seoul, Korea, MizMedi Hospital—Seoul National University, Seoul, Korea, National Centre for Biological Sciences/Tata Institute of Fundamental Research, Bangalore, India, Pochon CHA University, Seoul, Korea, Reliance Life Sciences, Mumbai, India, Technion University, Haifa, Israel, University of California, San Francisco, Calif., and Wisconsin Alumni Research Foundation, Madison, Wis. In addition, examples of embryonic stem cells are described in the following U.S. patents and published patent applications: U.S. Pat. Nos. 6,245,566; 6,200,806; 6,090,622; 6,331,406; 6,090,622; 5,843,780; 20020045259; 20020068045. In preferred embodiments, the human ES cells are selected from the list of approved cell lines provided by the National Institutes of Health and accessible at http://escr.nih.gov. Examples of human adult stem cells include those described in the following U.S. patents and patent applications: U.S. Pat. Nos. 5,486,359; 5,766,948; 5,789,246; 5,914,108; 5,928,947; 5,958,767; 5,968,829; 6,129,911; 6,184,035; 6,242,252; 6,265,175; 6,387,367; 20020016002; 20020076400; 20020098584; and, for example, in the PCT application WO 0111011. In certain embodiments, a suitable stem cell may be derived from a cell fusion or dedifferentiation process, such as described in the following US patent application: 20020090722, and in the following PCT applications: WO200238741, WO0151611, WO9963061, WO9607732.

In some preferred embodiments, a stem cell should be compliant with good tissue practice guidelines set for the by the U.S. Food and Drug Administration (FDA) or equivalent regulatory agency in another country. Methods to develop such a cells may include donor testing, and avoidance of exposure to non-human cells and products during derivation of the stem cells.

In certain preferred embodiments, stem cells may be hematopoietic or mesenchymal stem cells, such as stem cell populations derived from adult human bone marrow. Recent studies suggest that marrow-derived hematopoietic (HSCs) and mesenchymal stem cells (MSCs), which are readily isolated, have a broader differentiation potential than previously recognized. Many purified HSCs not only give rise to all cells in blood, but can also develop into cells normally derived from endoderm, like hepatocytes (Krause et al., 2001, Cell 105: 369-77; Lagasse et al., 2000 Nat Med 6: 1229-34). In at least one report (Lagasse et al, 2000 Nat Med 6: 1229-34), the possibility of somatic cell fusion was ruled out. MSCs appear to be similarly multipotent, producing progeny that can, for example, express neural cell markers (Pittenger et al., 1999 Science 284: 143-7; Zhao et al., 2002 Exp Neurol 174: 11-20).

In certain embodiments, stem cells are derived from an autologous source or an HLA-type matched source. For example, HSCs may be obtained from the bone marrow of a subject in need of ex vivo cell therapy and cultured by a method described herein to generate an autologous cell compositions. Other sources of stem cells are easily obtained from a subject, such as stem cells from muscle tissue, stem cells from skin (dermis or epidermis) and stem cells from fat. Stem cell compositions may also be derived from banked stem cell sources, such as banked amniotic epithelial stem cells or banked umbilical cord blood cells.

Stem cells may also be crude or fractionated bone marrow-derived cells (“BMDCs”). BMDCs may be obtained from any stage of development of the donor individual, including prenatal (e.g., embryonic or fetal), infant (e.g., from birth to approximately three years of age in humans), child (e.g. from about three years of age to about 13 years of age in humans), adolescent (e.g., from about 13 years of age to about 18 years of age in humans), young adult (e.g., from about 18 years of age to about 35 years of age in humans), adult (from about 35 years of age to about 55 years of age in humans) or elderly (e.g., from about 55 years and beyond of age in humans).

In some embodiments, the BMDCs are transfected and administered as unfractionated bone marrow. Bone marrow may be fractionated to enrich for certain BMDCs prior to administration. Methods of fractionation are well known in the art, and generally involve both positive selection (i.e., retention of cells based on a particular property) and negative selection (i.e., elimination of cells based on a particular property). As will be apparent to one of skill in the art, the particular properties (e.g., surface markers) that are used for positive and negative selection will depend on the species of the donor bone marrow-derived cells.

When the donor bone marrow-derived cells are human, there are a variety of methods for fractionating bone marrow and enriching bone marrow-derived cells. A subpopulation of BMDCs includes cells, such as certain hematopoietic stem cells that express CD34, and/or Thy-1. Depending on the cell population to be obtained, negative selection methods that remove or reduce cells expressing CD3, CDIO, CDIlb, CD14, CD16, CD15, CD16, CD19, CD20, CD32, CD45, CD45R/B220, Ly6G, and/or TER-119 may be employed. When the donor BMDCs are not autologous, it is preferred that negative selection be performed on the cell preparation to reduce or eliminate differentiated T cells, thereby reducing the risk of graft versus host disease.

Cells will generally derive from verterbrates, particularly mammals. Examples of vertebrate animals include fish, mammal, cattle, goat, pig, sheep, rodent, hamster, mouse, rat, primate, and human.

Invertebrate animals include nematodes, other worms, drosophila, and other insects. Representative generae of nematodes include those that infect animals (e.g., Ancylostoma, Ascaridia, Ascaris, Bunostomum, Caenorhabditis, Capillaria, Chabertia, Cooperia, Dictyocaulus, Haernonchus, Heterakis, Nematodirus, Oesophagostomum, Ostertagia, Oxyuris, Parascaris, Strongylus, Toxascaris, Trichuris, Trichostrongylus, Tflichonema, Toxocara, Uncinaria) and those that infect plants (e.g., Bursaphalenchus, Criconerriella, Diiylenchus, Ditylenchus, Globodera, Helicotylenchus, Heterodera, Longidorus, Melodoigyne, Nacobbus, Paratylenchus, Pratylenchus, Radopholus, Rotelynchus, Tylenchus, and Xiphinerna). Representative orders of insects include Coleoptera, Diptera, Lepidoptera, and Homoptera.

As will be apparent to one of skill in the art, it may be desirable to subject the recipient to an ablative regimen prior to administration of the shRNA transfected cells. Ablative regimens may involve the use of gamma radiation and/or cytotoxic chemotherapy to reduce or eliminate endogenous stem cells, such as hematopoietic stem cells and precursors. A wide variety of ablative regimens using chemotherapeutic agents are known in the art, including the use of cyclophosphamide as a single agent (50 mg/kg q day×4), cyclophosphamide plus busulfan and the DACE protocol (4 mg decadron, 750 mg/m2 Ara-C, 50 mg/in 2carboplatin, 50 mg/m2 etoposide, q 12h×4 IV). Additionally, gamma radiation may be used (e.g. 0.8 to 1.5 kGy, midline doses) alone or in combination with chemotherapeutic agents. In accordance with standard practice in the art, when chemotherapeutic agents are administered, it is preferred that the be administered via an intravenous catheter or central venous catheter to avoid adverse affects at the injection site(s).

### 4. Illustrative Uses

A. Methods of Treatment

In certain aspects, the invention provides methods of treating a disorder in a subject by introducing cells comprising a shRNA expression construct. In accordance with the methods disclosed herein, the shRNA may be reliably expressed in vivo in a variety of cell types. In certain embodiments the cells are administered in order to treat a condition. There are a variety of mechanisms by which shRNA expressing cells may be useful for treating a condition. For example, a condition may be caused in part by a population of cells expressing an undesirable gene. These cells may be ablated and replaced with administered cells comprising shRNA—that decreases expression of the undesirable gene; alternatively, the diseased cells may be competed away by the administered cells, without need for ablation. As another example, a condition may be caused by a deficiency in a secreted factor. Amelioration of such a disorder may be achieved by administering cells expressing a shRNA that indirectly stimulates production of the secreted factor, e.g., by inhibiting expression of an inhibitor.

A shRNA may be targeted to essentially any gene, the decreased expression of which may be helpful in treating a condition. The target gene participate in a disease process in the subject. The target gene may encode a host protein that is co-opted by a virus during viral infection, such as a cell surface receptor to which a virus binds while infecting a cell. HIV binds to several cell surface receptors, including CD4 and CXCR5. The introduction of HSCs or other T cell precursors carrying an shRNA directed to an HIV receptor or coreceptor is expected to create a pool of resistant T cells, thereby ameliorating the severity of the HIV infection. Similar principles apply to other viral infections.

Immune rejection is mediated by recognition of foreign Major Histocompatibility Complexes. Where heterologous cells are to be administered to a subject, the cells may be transfected with shRNAs that target any MHC components that are likely to be recognized by the host immune system.

In many embodiments, the shRNA transfected cells will achieve beneficial results by partially or wholly replacing a population of diseased cells in the subject. The transfected cells may autologous cells derived from cells of the subject, but carrying a shRNA that confers beneficial effects.

B. Screening Assays

One utility of the present invention is as a method of identifying gene function in an organism, especially higher eukaryotes, comprising the use of double-stranded RNA to inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity. The invention could be used in determining potential targets for pharmaceuticals, understanding normal and pathological events associated with development, determining signaling pathways responsible for postnatal development/aging, and the like. The increasing speed of acquiring nucleotide sequence information from genomic and expressed gene sources, including total sequences for mammalian genomes, can be coupled with the invention to determine gene function in a cell or in a whole organism. The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects.

A simple assay would be to inhibit gene expression according to the partial sequence available from an expressed sequence tag (EST). Functional alterations in growth, development, metabolism, disease resistance, or other biological processes would be indicative of the normal role of the EST's gene product.

The ease with which the dsRNA construct can be introduced into an intact cell/organism containing the target gene allows the present invention to be used in high throughput screening (HTS). For example, duplex RNA can be produced by an amplification reaction using primers flanking the inserts of any gene library derived from the target cell or organism. Inserts may be derived from genomic DNA or mRNA (e.g., cDNA and cRNA). Individual clones from the library can be replicated and then isolated in separate reactions, but preferably the library is maintained in individual reaction vessels (e.g., a 96 well microtiter plate) to minimize the number of steps required to practice the invention and to allow automation of the process.

In an exemplary embodiment, the subject invention provides an arrayed library of RNAi constructs. The array may be in the form of solutions, such as multi-well plates, or may be “printed” on solid substrates upon which cells can be grown. To illustrate, solutions containing duplex RNAs that are capable of inhibiting the different expressed genes can be placed into individual wells positioned on a microtiter plate as an ordered array, and intact cells/organisms in each well can be assayed for any changes or modifications in behavior or development due to inhibition of target gene activity.

In certain aspects, the invention provides methods for evaluating gene function in vivo. A cell containing an shRNA expression construct designed to decrease expression of a target gene may be introduced into an animal and a phenotype may be assessed to determine the effect of the decreased gene expression. An entire animal may be generated from cells (e.g., ES cells) containing an shRNA expression construct designed to decrease expression of a target gene. A phenotype of the transgenic animal may be assessed.

The animal may be essentially any experimentally tractable animal, such as a non-human primate, a rodent (e.g., a mouse), a lagomorph (e.g., a rabbit), a canid (e.g. a domestic dog), a feline (e.g., a domestic cat). In general, animals with complete or near complete genome projects are preferred.

A phenotype to be assessed may be essentially anything of interest. Quantitating the tendency of a stem cell to contribute to a particular tissue or tumor is a powerful method for identifying target genes that participate in stem cell differentiation and in tumorigenic and tumor maintenance processes. Phenotypes that have relevance to a disease state may be observed, such as susceptibility to a viral, bacterial or other infection, insulin production or glucose homeostasis, muscle function, neural regeneration, production of one or more metabolites, behavior patterns, inflammation, production of autoantibodies, obesity, etc.

A panel of shRNAs that affect target gene expression by varying degrees may be used, and phenotypes may be assessed. In particular, it may be useful to measure any correlation between the degree of gene expression decrease and a particular phenotype.

A heterogeneous pool of shRNA constructs may be introduced into cells, and these cells may be introduced into an animal. In an embodiment of this type of experiment, the cells will be subjected to a selective pressure and then it will be possible to identify which shRNAs confer resistance or sensitivity to the selective pressure. The selective pressure may be quite subtle or unintentional, for example, mere engraftment of transfected HSCs may be a selective pressure, with some shRNAs interfering with engraftment and others promoting engraftment. Development and differentiation may be viewed as a “selective pressure”, with some shRNAs modulating the tendency of certain stem cells to differentiate into different subsets of progeny. Treatment with a chemotherapeutic agent may be used as selective pressure, as described below. The heterogeneous pool of shRNAs may be obtained from a library, and in certain preferred embodiments, the library is a barcoded library, permitting rapid identification of shRNA species.

In certain aspects, the invention provides methods for identifying genes that affect the sensitivity of tumor cells to a chemotherapeutic agent. The molecular mechanisms that underlie chemoresistance in human cancers remain largely unknown. While various anticancer agents clearly have different mechanisms of action, most ultimately either interfere with DNA synthesis or produce DNA damage. This, in turn, triggers cellular checkpoints that either arrest cell proliferation to allow repair or provoke permanent exit from the cell cycle by apoptosis or senescence.

In certain embodiments, a method comprises introducing into a subject a transfected stem cell comprising a nucleic acid construct encoding an shRNA, wherein the shRNA is complementary to at least a portion of a target gene, wherein the transfected stem cell exhibits decreased expression of the target gene, and wherein the transfected stem cell gives rise to a transfected tumor cell in vivo. For example, the stem cell may be derived from an animal that has a genetic predisposition to tumorigenesis, such as an oncogene over-expressing animal (e.g. Eμ-myc mice) or a tumor suppressor knockout (e.g., p53 −/− animal). Alternatively, an animal comprising the stem cells may be exposed to carcinogenic conditions such that tumors comprising cells derived from the stem cells are generated. An animal having tumors may be treated with a chemotherapeutic or other anti-tumor regimen, and the effect of this regimen on cells expressing the shRNA may be evaluated. An shRNA that is overrepresented following anti-tumor therapy is likely to be targeted against a gene that confers sensitivity. An shRNA that is underrepresented following anti-tumor therapy is likely to be targeted against a gene that confers resistance. An shRNA that is underrepresented may be developed for use as a co-therapeutic to be co-administered with the chemotherapeutic agent in question and suppress resistance.

Overrepresentation and underrepresentation are generally comparative terms, and determination of these parameters will generally involve comparison to a control or benchmark. A comparison may simply be to the same animal prior to chemotherapy administration. A comparison may also be to a control subject that has not received the chemotherapeutic agent. A comparison may be to an average of multiple other shRNA trials. Any control need not be contemporaneous with the experiment, although the protocol should be substantially the same.

This technique may be performed on individual shRNAs (see e.g., BIM shRNA, in the Examples below). The technique may also be adopted for highly parallel screening. For example, a method may comprise introducing into a subject a plurality of transfected stem cells, wherein each transfected stem cell comprises a nucleic acid construct comprising a representative shRNA of an shRNA library, and wherein a representative shRNA of an shRNA library is complementary to at least a portion of a representative target gene, wherein a plurality of the transfected stem cells exhibits decreased expression of a representative target gene, and wherein a plurality of the transfected stem cells gives rise to transfected tumor cells in vivo. Notably, it is not necessary or expected that every shRNA is different or that every transfected cell will become part of a tumor. Once tumors have been generated, a chemotherapeutic or other anti-tumor regimen may be administered, and the overrepresentation or underrepresentation of shRNA species may be evaluated. In certain preferred embodiments, each representative shRNA is associated with a distinguishable tag that permits rapid identification of each shRNA. For example, shRNAs may be obtained from a shRNA library that is barcoded.

Certain methods described herein take advantage of the fact that large numbers of cancer cells (e.g., lymphoma cells) can be isolated from affected mice and transplanted into syngeneic, immunocompetent recipients to create a lymphoma that is virtually indistinguishable from the spontaneous disease. This allows in vitro manipulation of tumor cells to create potentially chemoresistant variants that can be analyzed in vivo. In certain exemplary embodiments, the invention exploits advantages of the Eμ-myc system to undertake an unbiased search for genetic alterations that can confer resistance to chemotherapeutics, such as the widely used alkylating agent, CTX.

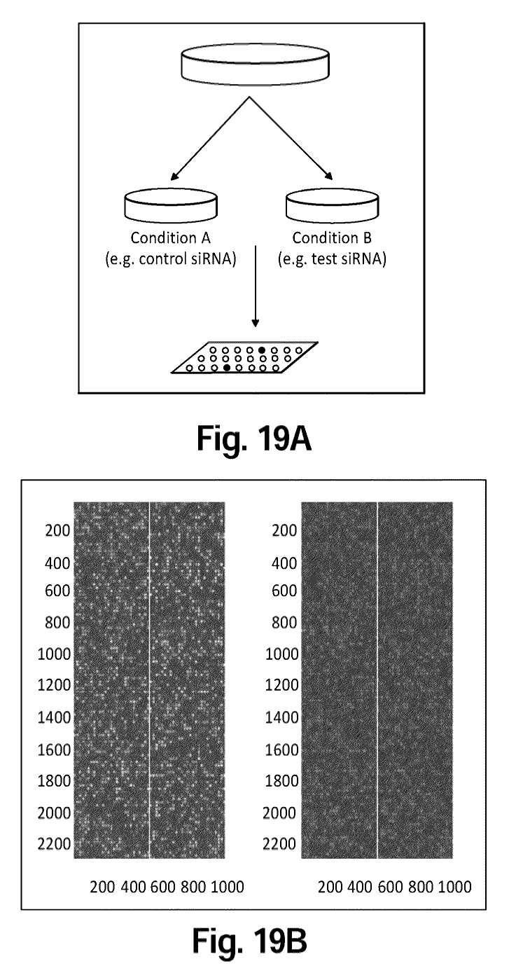

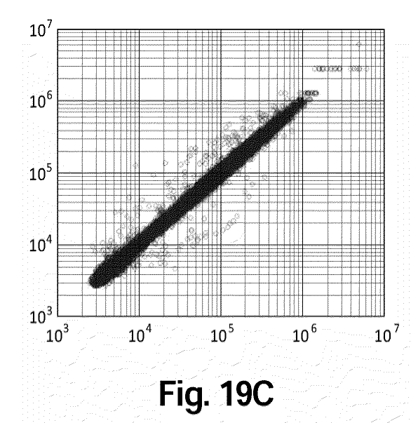

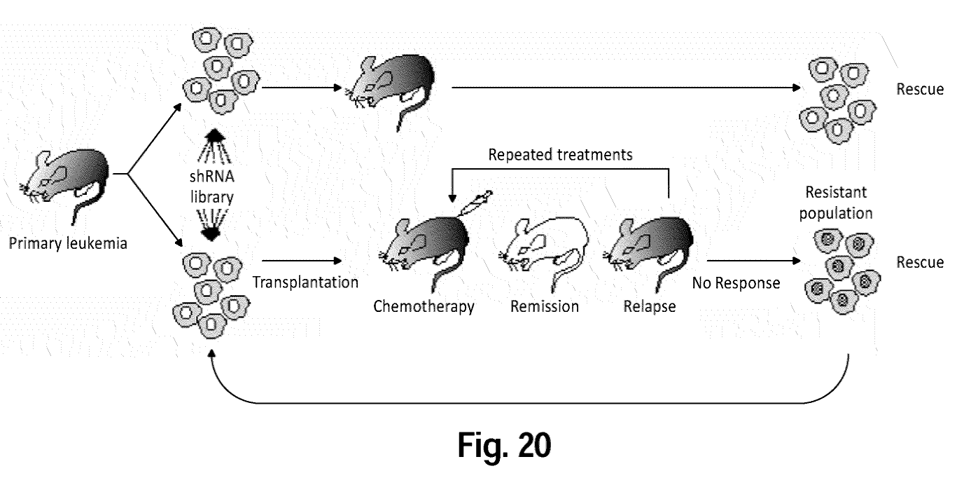

The following is an outline of an example of a screen to identify genes that confer resistance to CTX using an unbiased, genetic approach. An overview of the screen is diagrammed in FIG. 19. Populations of isolated lymphoma cells from the Eμ-myc mouse receive pools of sequence verified shRNAs that specifically target murine genes. Engineered cells are introduced into immunocompetent, syngeneic recipient animals. Upon the appearance of tumors, the animals are be treated with CTX. In each case, the time of remission is measured, and, upon relapse, the animals undergo a second round of treatment. After two rounds of therapy, the shRNA resident in resistant populations are identified and transferred into fresh populations of lymphoma cells, which are transplanted into naïve animals. After the appropriate number of selection cycles, individual shRNAs that are capable of conferring drug resistance are obtained.

C. Barcoding Methods

In certain embodiments, an expression construct that transcribes an RNAi species, e.g., a dsRNA or hairpin RNA, can include a barcode sequence. For those embodiments in which the RNAi constructs are provided as a variegated library for generating different RNAi species against a variety of different target sequence, each member (e.g., each unique target sequence) of the library can include a distinct barcode sequence such that that member of the library can be later identified if isolated individually or as part of an enriched population of RNAi constructs.

For example, two methods for determining the identity of the barcode sequence are by chemical cleavage, as disclosed by Maxim and Gilbert (1977), and by chain extension using ddNTPs, as disclosed by Sanger et al. (1977). In other embodiments, the sequence can be obtained by techniques utilizing capillary gel electrophoresis or mass spectroscopy. See, for example, U.S. Pat. No. 5,003,059.

Alternatively, another method for determining the identity of a barcode sequence is to individually synthesize probes representing each possible sequence for each character position of a barcode sequence set. Thus, the entire set would comprise every possible sequence within the barcode sequence portion or some smaller portion of the set. By various deconvolution techniques, the identity of the probes which specifically anneal to the barcode sequence sequences can be determined. An exemplary procedure would be to synthesize one or more sets of nucleic acid probes for detecting barcode sequence sequences simultaneously on a solid support. Preferred examples of a solid support include a plastic, a ceramic, a metal, a resin, a gel, and a membrane. A more preferred embodiment comprises a two-dimensional or three-dimensional matrix, such as a gel, with multiple probe binding sites, such as a hybridization chip as described by Pevzner et al. (J. Biomol. Struc. & Dyn. 9:399-410, 1991), and by Maskos and Southern (Nuc. Acids Res. 20:1679-84, 1992).

Hybridization chips can be used to construct very large probe arrays which are subsequently hybridized with a target nucleic acid. Analysis of the hybridization pattern of the chip provides an immediate fingerprint identification of the barcode sequence sequence. Patterns can be manually or computer analyzed, but it is clear that positional sequencing by hybridization lends itself to computer analysis and automation. Algorithms and software have been developed for sequence reconstruction which are applicable to the methods described herein (Drmanac et al., (1992) Electrophoresis 13:566-73; P. A. Pevzner, J. Biomol. Struc. & Dyn. 7:63-73, 1989).

For example, the identity of the barcode sequence sequence can be determined by annealing a solution of test sample nucleic acid including one or more barcode sequence sequences to a fixed array of character detection oligonucleotides (barcode sequence probes), where each column in the array preferably codes for one character of the barcode sequence. Each fixed oligonucleotide has a nucleotide base sequence that is complementary to the nucleotide base sequence of a single character. Either the test sample nucleic acid or the fixed oligonucleotides can be labeled in such a fashion to permit read-out upon hybridization, e.g., by radioactive labeling or chemiluminescent labeling. Test nucleic acid can be labeled, for example, by using PCR to amplify the identification region of a DNA pool under test with PCR primers that are radioactive or chemiluminescent. Preferred detectable labels include a radioisotope, a stable isotope, an enzyme, a fluorescent chemical, a luminescent chemical, a chromatic chemical, a metal, an electric charge, or a spatial structure. There are many procedures whereby one of ordinary skill can incorporate detectable label into a nucleic acid.

For example, enzymes used in molecular biology will incorporate radioisotope labeled substrate into nucleic acid. These include polymerases, kinases, and transferases. The labeling isotope is preferably, ³²P, ³⁵S, ¹⁴C, or ¹²⁵I.

Other, more advanced methods of detection include evanescent wave detection of surface plasmon resonance of thin metal film labels such as gold, by, for example, the BIAcore sensor sold by Pharmacia, or other suitable biosensors. An exemplary plasmon resonance technique utilizes a glass slide having a first side on which is a thin metal film (known in the art as a sensor chip), a prism, a source of monochromatic and polarized light, a photodetector array, and an analyte channel that directs a medium suspected of containing an analyte, in this case a barcode sequence-containing nucleic acid, to the exposed surface of the metal film. A face of the prism is separated from the second side of the glass slide (the side opposite the metal film) by a thin film of refractive index matching fluid. Light from the light source is directed through the prism, the film of refractive index matching fluid, and the glass slide so as to strike the metal film at an angle at which total internal reflection of the light results, and an evanescent field is therefore caused to extend from the prism into the metal film. This evanescent field can couple to an electromagnetic surface wave (a surface plasmon) at the metal film, causing surface plasmon resonance. When an array of barcode sequence probes are attached to the sensor chip, the pattern of annealing to barcode sequence sequences produces a detectable pattern of surface plasmon resonance on the chip.

The pattern of annealing, e.g., of selective hybriziation, of the labeled test DNA to the oligonucleotide array or the test DNA to the labeled oligonucleotide array permits the barcode sequence present in the original DNA clone to be directly read out. The detection array can include redundant oligonucleotides to provide integrated error checking.

In general, the hybridization will be carried out under conditions wherein there is little background (non-specific) hybridization, e.g., the background level is at least one order of magnitude less than specific binding, and even more preferably, at least two, three or four orders of magnitude less.

Additionally, the array can contain oligonucleotides that are known not to match any barcode sequence in the library as a negative control, and/or oligonucleotides that are known to match all barcode sequences, e.g., primer flanking sequence, as a positive control.

### 5. Cell Delivery Systems

In certain embodiments, the invention provides a composition formulated for administration to a patient, such as a human or veterinary patient. A composition so formulated may comprise a stem cell comprising a nucleic acid construct encoding an shRNA designed to decrease the expression of a target gene. A composition may also comprise a pharmaceutically acceptable excipient. Essentially any suitable cell may be used, included cells selected from among those disclosed herein. Transfected cells may also be used in the manufacture of a medicament for the treatment of subjects. Examples of pharmaceutically acceptable excipients include matrices, scaffolds or other substrates to which cells may attach (optionally formed as solid or hollow beads, tubes, or membranes), as well as reagents that are useful in facilitating administration (e.g. buffers and salts), preserving the cells (e.g. chelators such as sorbates, EDTA, EGTA, or quaternary amines or other antibiotics), or promoting engraftment.

Cells may be encapsulated in a membrane or in a microcapsule. Cells may be placed in microcapsules composed of alginate or polyacrylates. (Lim et al. (1980) Science 210:908; O'Shea et al. (1984) Biochim. Biochys. Acta. 840:133; Sugamori et al. (1989) Trans. Am. Soc. Artif. Intern. Organs 35:791; Levesque et al. (1992) Endocrinology 130:644; and Lim et al. (1992) Transplantation 53:1180). Additional methods for encapsulating cells are known in the art. (Aebischer et al. U.S. Pat. No. 4,892,538; Aebischer et al. U.S. Pat. No. 5,106,627; Hoffman et al. (1990) Expt. Neurobiol. 110:39-44; Jaeger et al. (1990) Prog. Brain Res. 82:41-46; and Aebischer et al. (1991) J. Biomech. Eng. 113:178-183, U.S. Pat. No. 4,391,909; U.S. Pat. No. 4,353,888; Sugamori et al. (1989) Trans. Am. Artif. Intern. Organs 35:791-799; Sefton et al. (1987) Biotehnol. Bioeng. 29:1135-1143; and Aebischer et al. (1991) Biomaterials 12:50-55).

The site of implantation of insulin-producing cell compositions may be selected by one of skill in the art depending on the type of cell and the therapeutic objective. Exemplary implantation sites include intravenous or intraarterial administration, administration to the liver (via portal vein injection), the peritoneal cavity, the kidney capsule or the bone marrow.

### EXAMPLES

### Example 1

### Stable Introduction of shRNA-Transfected Cells into Mice

In this Example, Applicants demonstrate the introduction of an RNA interference construct into stem cells and the stable maintenance of an RNA interference-derived phenotype in vivo after cell implantation. The test system is the Eμ-myc transgenic mouse system established by Applicants; these mice overexpress the myc gene in B cell lineages and generate lymphoma-like tumors. Features of the Eμ-myc mouse model include: (i) Eμ-myc lymphomas recapitulate typical genetic and pathological features of human Non-Hodgkin's lymphomas; (ii) tumors arise with relatively short latency and high penetrance; (iii) tumor burden can be easily monitored by lymph-node palpation or blood smears; (iv) lymphomas are detectable long before the animal dies; (v) large numbers of pure tumor cells can be isolated from enlarged lymph-nodes for biochemical studies; (vi) therapy is performed in immunocompetent mice; and (vii) lymphoma cells can be cultured and transplanted into syngeneic, non-transgenic recipient mice. In addition, Applicants have developed methods for manipulating the genotype of Eμ-myc lymphomas, allowing the creation of tumors with defined genetic lesions and an assessment of the relationship of these to treatment responses. This also allows ‘tagging’ of tumor cells with fluorescent proteins and monitoring of tumor burden by in vivo imaging in live mice. Furthermore, Applicants have previously demonstrated that Myc-initiated lymphomas can be generated with different secondary lesions by (i) intercrossing to genetically engineered mice, (ii) rapidly transferring retroviral genes into established lymphomas, or (iii) retrovirally infecting hematopoietic stem cells prior to their propagation in myeloablated recipient mice. These different approaches can be combined in a way that lymphomas with multiple genotypes are rapidly produced. See, e.g., Schmitt, C. A., et al., A senescence program controlled by p53 and p16INK4a contributes to the outcome of cancer therapy. Cell, 2002. 109(3): p. 335-46; Schmitt, C. A., C. T. Rosenthal, and S. W. Lowe, Genetic analysis of chemoresistance in primary murine lymphomas. Nat Med, 2000. 6(9): p. 1029-35; Schmitt, C. A, Fridman, J. S., Yang, M., Baranov, E., Hoffman, R. M., and Lowe, S. W. Dissecting p53 tumor suppressor functions in vivo. Cancer Cell 2002. 1: p. 289-98.

Tumor cells which express exogenous genes may be generated by harvesting hematopoietic stem cells from Eμ-myc transgenic fetal livers and introducing various constructs using recombinant retroviruses. These cells are transplanted into multiple lethally irradiated recipient animals by tail vein injection. Applicants have shown that these mice develop B-cell tumors in an equivalent time frame to their non-transplanted counterparts (Schmitt et al., Cancer Cell 1:289-98 (2002)).

Applicants have previously published that Eμ-myc mice, which are p53−/−, develop tumors at an accelerated rate (Schmitt et al., Genes Dev. 13:2670-77 (1999)). Here applicants show that various p53 shRNAs introduced into a p53 +/+background can recapitulate the p53 −/− phenotype and accelerate tumor formation to varying degrees. Of note, applicants have shown that the acuteness of the phenotype is dependent on the hairpin applicants use. In essence, applicants can generate a panel of hairpins which result in a gradient of activity; fully functional, 75% functional, 50% functional and so forth. This type of panel is quite useful in analyzing a specific gene's contribution to the biology of a condition, such as a tumor. The biological activity of these shRNAs is further demonstrated by the lack of loss of heterozygosity (LOH) in p53 +/− Eμ-myc tumors expressing the short hairpins compared to 100% LOH in control tumors. Applicants have also been able to isolate cells from shRNA expressing tumors and re-transplant them into syngenic mice. The arising tumors continue to suppress p53 and are as aggressive as their p53−/− counterparts.

### Materials and Methods

Generation of p53 shRNA retroviruses-p53 hairpin oligos were designed using designated software found at the Cold Spring Harbor Laboratory website. The hairpins described in this application have the following sequence:

p53-1:
AAAAAGGTCTAAGTGGAGCCCTTCGAGTGTTAGAAGCTTGTGACACTCGG
AGGGCTTCACTTGGGCCCGGTGTTTCGTCCTTTCCACAA
AND,
p53-2:
AAAAAAAACATCCGACTGCGACTCCTCCATAGCAGCAAGCTTCCTGCCAT
GGAGGAGTCACAGTCGGATATCGGTGTTTCGTCCTTTCCACAA.

To generate hairpin sequences downstream of U6 promoter, PCR reactions were run using a pGEM U6 promotor template (provided by Greg Hannon), the p53 hairpin primers and a CACC-SP6 reverse primer with the following sequence: CACCGATTTAGGTGACACTATAG. The PCR conditions were the following: 100 ng pGEM U6 plasmid, 1 μM p53 hairpin primer, 1 μM SP6, 1× Perkin-Elmer PCR reaction buffer (with 15 mM MgCl2), 4% DMSO, 0.25 mM dNTPs and 5 Units of taq DNA polymerase. Reactions were run for 1×95 degrees for 5 minutes, 30 cycles of 95 degrees 30″, 55 degrees 30″ and 72 degrees 1′. PCR products were then blunted by incubating at 72 degrees for 10 minutes in the presence of 2 units of pfu DNA polymerase. PCR products were cloned directly into a pENTR/TOPO-D vector (Invitrogen), using the company specifications. Plasmids containing the PCR product were cut with EcoRV and gel extracted. The cut plasmid was placed into a “Gateway™” reaction (Invitrogen) reaction with a retroviral vector containing a “Gateway™ destination cassette” and the Gateway™ BP clonase enzyme mix. The reaction was performed as specified in the Gateway™ BP clonase enzyme product literature. Retroviral vectors containing destination cassettes were created as follows: pBabe Puro was cut with Nhe1 and a linear reading frame cassette A (Gibco/Brl) fragment was blunt-end ligated into the cut vector in the 3′ LTR. MSCV puro (Clontech) was cut with Hpa1 and a linear reading frame cassette A was blunt-end ligated into the cut vector upstream of the PGK promoter.

Retroviral Infection of Stem Cells—Stem cells were isolated from the fetal livers of EμMyc transgenic mice as described (Schmitt et al, Cancer Cell 1(2):289-98). Genotyping for the presence of the EμMyc transgene was done as described. Retroviral infection was performed using vectors p53-A, p53-B and p53-C as described (Schmitt et al., Cancer Cell. 2002 (3):289-98).

Tumor Analysis—Tumor burden was monitored externally by lymph node palpation. The presence of the hairpin DNA in tumors was confirmed by performing the same PCR reaction described above, replacing the pGEM U6 template with 100 ng of tumor DNA. H&E staining of lymph nodes, lung and spleen in recipient animals was performed to confirm the presence of a pathology consistent with B-cell lymphoma. TUNEL assays were performed to determine the level of in-tumor apoptosis.

LOH Analysis—Retroviral infection of p53+/− stem cells was performed using vectors p53-A, p53-B and p53-C as described (Schmitt et al, Cancer Cell, 1(3):289-98 (2002)). The genotype of the recipient stem cells and the resulting DNA was performed as described.

### REFERENCES

Schmitt, C. A., Fridman, J. S., Yang, M., Baranov, E., Hoffman, R. M., and Lowe, S. W. (2002). Dissecting p53 tumor suppressor functions in vivo. Cancer Cell 1:289-98.
Schmitt, C. A., McCurrach, M. E., de Stanchina, E., Wallace-Brodeur, R., and Lowe, S. W. 1999. INK4a/ARF mutations accelerate lymphomagenesis and promote chemoresistance by disabling p53. Genes Dev. 13:2670-77.
Short hairpin RNAs (shRNAs) induce sequence-specific silencing in mammalian cells. Paddison P J, Caudy A A, Bernstein E, Hannon G J, Conklin D S. Genes Dev 2002 Apr. 15; 16(8):948-58.
RNA as a target of double-stranded RNA-mediated genetic interference in Caenorhabditis elegans. Montogomery M K, Xu S, Fire A. Proc Natl Acad Sci USA 1998 Dec. 22; 95(26):15502-7.
Potent and specific genetic interference by double-stranded RNA in Caenorhabditis elegans. Fire A, Xu S, Montgomery M K, Kostas S A, Driver S E, Mello C C. Nature 1998 Feb. 19; 391(6669):806-11.

### Example 2

### Germline Transmission of RNAi in Mice

MicroRNA molecules (miRNAs) are small, noncoding RNA molecules that have been found in a diverse array of eukaryotes, including mammals. miRNA precursors share a characteristic secondary structure, forming short ‘hairpin’ RNAs. Genetic and biochemical studies have indicated that miRNAs are processed to their mature forms by Dicer, an RNAse III family nuclease, and function through RNA-mediated interference (RNAi) and related pathways to regulate the expression of target genes (Hannon 2002, Nature 418: 244-251; Pasquinelli et al. 2002, Annu. Rev. Cell. Dev. Biol. 18: 495-513). Recently, applicants and others have remodeled miRNAs to permit experimental manipulation of gene expression in mammalian cells and have dubbed these synthetic silencing triggers ‘short hairpin RNAs’ (shRNAs) (Paddison et al. 2002, Cancer Cell 2: 17-23). Silencing by shRNAs requires the RNAi machinery and correlates with the production of small interfering RNAs (siRNAs), which are a signature of RNAi.

Expression of shRNAs can elicit either transient or stable silencing, depending upon whether the expression cassette is integrated into the genome of the recipient cultured cell (Paddison et al. 2002, Cancer Cell 2: 17-23). shRNA expression vectors also induce gene silencing in adult mice following transient delivery (Lewis et al. 2002, Nat. Genet. 32: 107-108; McCaffrey et al. 2002, Nature 418: 38-39). However, for shRNAs to be a viable genetic tool in mice, stable manipulation of gene expression is essential. As shown in Example 1, Applicants have demonstrated long-term suppression of gene expression in vivo following retroviral delivery of shRNA-expression cassettes to hematopoietic stem cells. Here Applicants demonstrated a methodology by which shRNA-expression cassettes that are passed through the mouse germline can enforce heritable gene silencing.

Applicants began by taking standard transgenesis approaches (Gordon et al. 1993, Methods Enzymol. 225: 747-771) using shRNAs directed against a variety of targets with expected phenotypes, including the genes encoding tyrosinase (albino), myosin VIIa (shaker), Bmp-5 (crinkled ears), Hox a-10 (limb defects), homogentisate 1,2,-dioxygenase (urine turns black upon exposure to air), Hairless (hair loss) and melanocortin 1 receptor (yellow). Three constructs per gene were linearized and injected into pronuclei to produce transgenic founder animals. Although applicants noted the presence of the transgene in some animals, virtually none showed a distinct or reproducible phenotype that was expected for a hypomorphic allele of the targeted gene.

Therefore, applicants decided to take another approach: verifying the presence of the shRNA and its activity toward a target gene in cultured embryonic stem (ES) cells and then asking whether those cells retained suppression in a chimeric animal in vivo. Applicants also planned to test whether such cells could pass a functional RNAi-inducing construct through the mouse germline. For these studies, applicants chose to examine a novel gene, Neil1, which is proposed to have a role in DNA repair. Oxidative damage accounts for 10,000 DNA lesions per cell per day in humans and is thought to contribute to carcinogenesis, aging and tissue damage following ischemia (Ames et al. 1993, Proc. Natl. Acad. Sci. USA 90: 7915-7922; Jackson et al. 2001, Mutat. Res. 477: 7-21). Oxidative DNA damage includes abasic sites, strand breaks and at least 20 oxidized bases, many of which are cytotoxic or pro-mutagenic (Dizdaroglu et al. 2002, Free Radic. Biol. Med. 32: 1102-1115). DNA N-glycosylases initiate the base excision repair pathway by recognizing specific bases in DNA and cleaving the sugar base bond to release the damaged base (David et al. 1998, Chem. Rev. 98: 1221-1262).

The Neil genes are a newly discovered family of mammalian DNA N-glycosylases related to the Fpg/Nei family of proteins from Escherichia coli (Hazra et al. 2002, Proc. Natl. Acad. Sci. USA 99: 3523-3528; Bandaru et al. 2002, DNA Repair 1: 517-529). Neil1 recognizes and removes a wide spectrum of oxidized pyrimidines and ring-opened purines from DNA, including thymine glycol (Tg), 2,6-diamino-4-hydroxy-5-formamidopyrimidine (FapyG) and 4,6-diamino-5-formidopyrimidine (FapyA). Tg, FapyG and FapyA are among the most prevalent oxidized bases produced by ionizing radiation (Dizdaroglu et al. 2002, Free Radic. Biol. Med. 32: 1102-1115) and can block replicative DNA polymerases, which can, in turn, cause cell death (Asagoshi et al. 2002, J. Biol. Chem. 277: 14589-14597; Clark et al. 1989, Biochemistry 28: 775-779).

The Nth1 and Ogg1 glycosylases each remove subsets of oxidized DNA bases that overlap with substrates of Neil1 (Nishimura 2002, Free Radic. Biol. Med. 32: 813-821; Asagoshi et al. 2000, Biochemistry 39: 11389-11398; Dizdaroglu et al. 1999, Biochemistry 38: 243-246). However, mice with null mutations in either Nth1 (Ocampo et al. 2002, Mol. Cell. Biol. 22: 6111-6121; Takao et al. 2002, EMBO J. 21: 3486-3493) or Ogg1 (Klungland et al. 1999, Proc. Natl. Acad. Sci. USA 96: 13300-13305; Minowa et al. 2000, Proc. Natl. Acad. Sci. USA 97: 4156-4161) are viable, raising the possibility that Neil1 activity tempers the loss of Nth1 or Ogg1. Recently, a residual Tg-DNA glycosylase activity in Nth1^{-/-} mice has been identified as Neil1 (Takao et al. 2002, J. Biol. Chem. 277: 42205-42213).

Applicants constructed a single shRNA expression vector targeting a sequence near the 5′ end of the Neil1 coding region. This vector was introduced into mouse embryonic stem cells by electroporation, and individual stable integrants were tested for expression of the Neil1 protein (see the weblink: http://www.cshl.edu/public/SCIENCE/hannon.html for detailed procedures). The majority of cell lines showed an ˜80% reduction in Neil1 protein, which correlated with a similar change in levels of Neil1 mRNA. These cells showed an approximately two-fold increase in their sensitivity to ionizing radiation, consistent with a role for Neil1 in DNA repair. Two independent ES cell lines were injected into BL/6 blastocysts, and several high-percentage chimeras were obtained. These chimeras were out-crossed, and germline transmission of the shRNA-expression construct was noted in numerous F₁ progeny (13/27 for one line and 12/26 for the other).

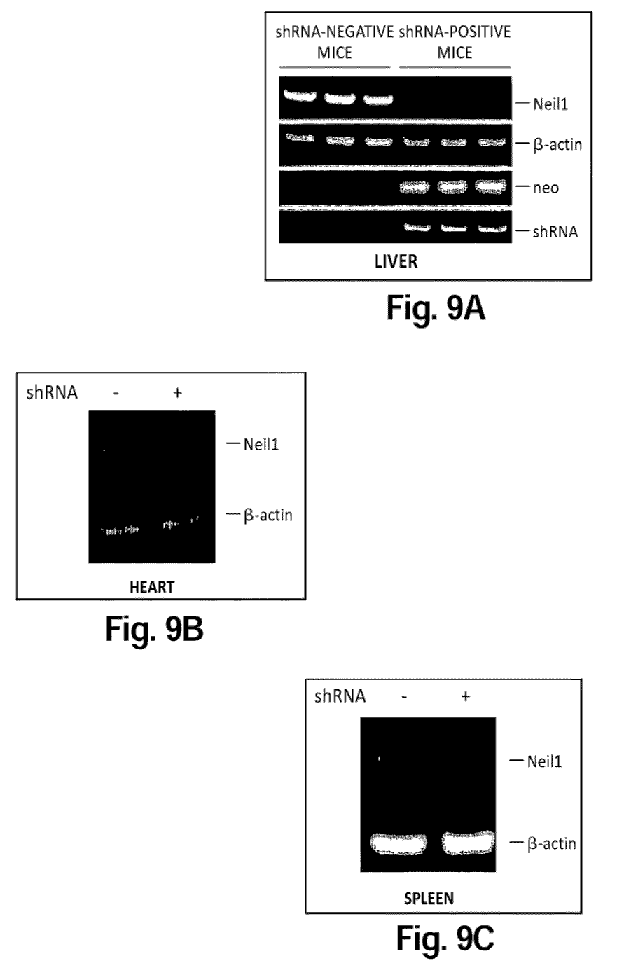

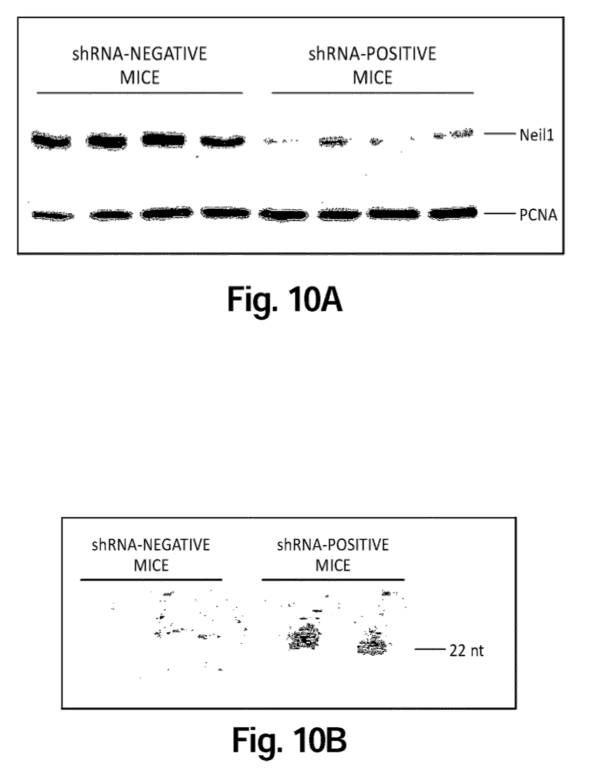

To determine whether the silencing of Neil1 that had been observed in ES cells was transmitted faithfully, applicants examined Neil1 mRNA and protein levels. Both were reduced by approximately the same extent that had been observed in the engineered ES cells (FIGS. 9, 10). Consistent with this having occurred through the RNAi pathway, applicants detected the presence of siRNAs corresponding to the shRNA sequence in F₁ animals that carry the shRNA expression vector but not in those that lack the vector (FIG. 10b).

The aforementioned data demonstrate that shRNAs can be used to create germline transgenic mice in which RNAi has silenced a target gene. These observations open the door to using of RNAi as a complement to standard knock-out methodologies and provide a means to rapidly assess the consequences of suppressing a gene of interest in a living animal. Coupled with activator-dependent U6 promoters, the use of shRNAs will ultimately provide methods for tissue-specific, inducible and reversible suppression of gene expression in mice.

### Example 3

### shRNA Modification of Stem Cells: Bim and Puma

Example 1, above, describes the use of p53 shRNA constructs to reduce p53 levels in hematopoietic stem cells. This reduction in p53 levels, in conjunction with Myc overexpression, was sufficient to produce tumor phenotypes in reconstituted recipient animals. Here, Applicants demonstrate the broad applicability of this technology for reducing gene expression in stem cells by targeting two additional putative tumor suppressors: Bim and Puma.

Bim and Puma shRNA constructs were created as described for the shp53 constructs. The primers used to create Bim shRNAs were:

mBim-1-
AAAAAAATCACACTCAGAACTCACACCAGAAGGCTCAAGCTTCAACCTTC
TGATGTAAGTTCTGAGTGTGACGGTGTTTCGTCCTTTCCACAA
mBim-2-
AAAAAAAAGAGTAGTCTTCAGCCTCGCAGTAATCACAAGCTTCTGATTAC
CGCGAGGCTGAAGACCACCCTCGGTGTTTCGTCCTTTCCACAA
mBim-3-
AAAAAAGAGATAGGGACCCCAAGCCTGAGCTGGAGCAAGCTTCCCCCAGC
TCAGGCCTGGGGCCCCTACCTCGGTGTTTCGTCCTTTCCACAA

The primers used to create Puma shRNAs were:

mPUMA-1-
AAAAAAGAGAGCCGCCCTCCTAGCATGCGCAGGCCCAAGCTTCGGCCCGC
GCACGCCAGGAGGGCAGCTCTCGGTGTTTCGTCCTTTCCACAA
mPUMA-2-
AAAAAAGGGACTCCAAGATCCCTGAGTAAGAGGAGCAAGCTTCCTCCCCT
TACCCAGGGATCCTGGAGCCCCGGTGTTTCGTCCTTTCCACAA
mPUMA-3-
AAAAAAGGGAGGGCTAAGGACCGTCCGAGCACGAGCAAGCTTCCCCGCGC
CCGGACGGTCCTCAGCCCTCCCGGTGTTTCGTCCTTTCCACAA

After PCR reactions using a U6 template (see Example 1), the resulting U6 shRNA PCR products were transferred into both MSCV Puro and MSCV Puro-IRES-GFP retroviral constructs. Virus generated from MSCV Puro Bim shRNA and MSCV Puro-IRES-GFP Puma shRNA constructs was used to infect Em-Myc hematopoietic stem cells. The infected stem cells were then used to reconstitute the hematopoictic system of irradiated recipient mice.

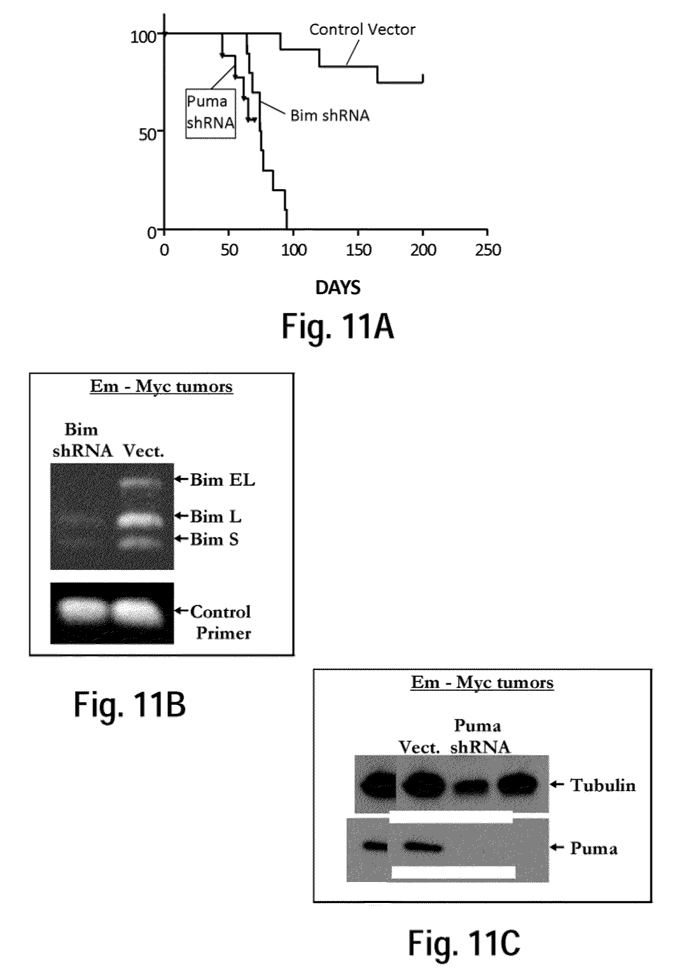

Mice receiving MSCV Puro Bim shRNA and MSCV Puro-IRES-GFP Puma shRNA developed lymphomas at a significantly higher penetrance and shorter onset time than mice receiving control vector (FIG. 11A). RT-PCR of total RNA was performed on tumors from mice receiving control or MSCV Puro Bim shRNA vectors, using the following primers:

| | |
|---|---|
| mBim5′-Xho1 | CCGCTCGAGGCCACCATGGCCAAGCAACCTTCTGATG |
| mBim3′-EcoRI | CCGGAATTCTCAATGCCTTCTCCATACCAGACG |

Tumors arising in mice receiving MSCV Puro Bim shRNA virus showed a nearly complete reduction in all Bim splice forms, while control tumors showed significant amount of Bim RNA (FIG. 11B). Western blots were performed on tumors from control vector and MSCV Puro-IRES-GFP Puma shRNA mice, using an Anti-Puma antibody (Axxora, LLC). Tumors arising in mice receiving MSCV Puro-IRES-GFP Puma shRNA virus showed a significant reduction in Puma expression relative to control-infected tumors (FIG. 11C).

These results establish that 1) stable RNAi in stem cells is possible for a wide variety of target genes, 2) shRNA constructs can produce stable phenotypes in recipient cells and 3) these constructs specifically repress their proposed targets.

### Example 4

### Modulating Chemotherapeutic Resistance in Stem Cells and Tumor Cells Using Stable RNAi

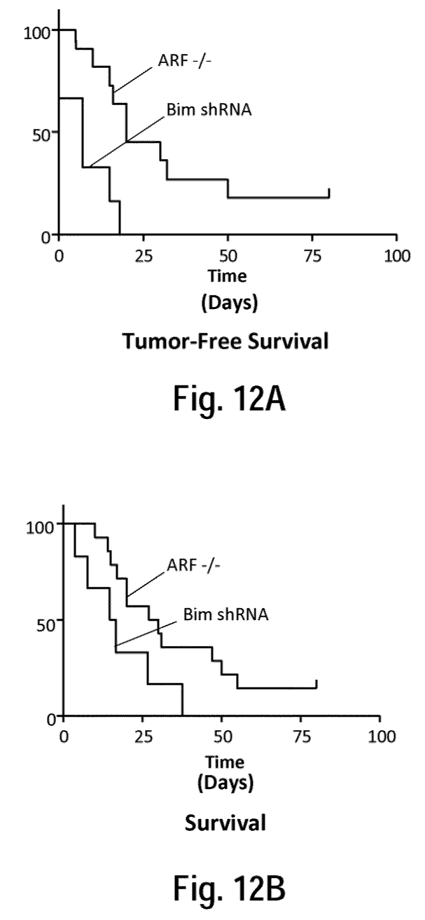

Bim plays a well-established role in antagonizing Bcl-2 function, and Bcl-2 overexpression has previously been shown to mediate chemotherapeutic resistance in vivo. To examine whether gene suppression by RNAi could affect treatment response, as well as tumor formation, we examined the response of tumors created with MSCV Puro Bim shRNAs to chemotherapy. Control and Bim shRNA tumors were treated with 10 mg/kg adriamycin and monitored for tumor-free survival by regular palpation and blood smears (see Schmitt et al., Cancer Cell 2002; Cell). Bim shRNA tumors showed a significant decrease in tumor free survival and time to death relative to control tumors (FIG. 12). Thus, stem cells engineered to express shRNAs can yield tumors with distinct chemotherapeutic sensitivities.

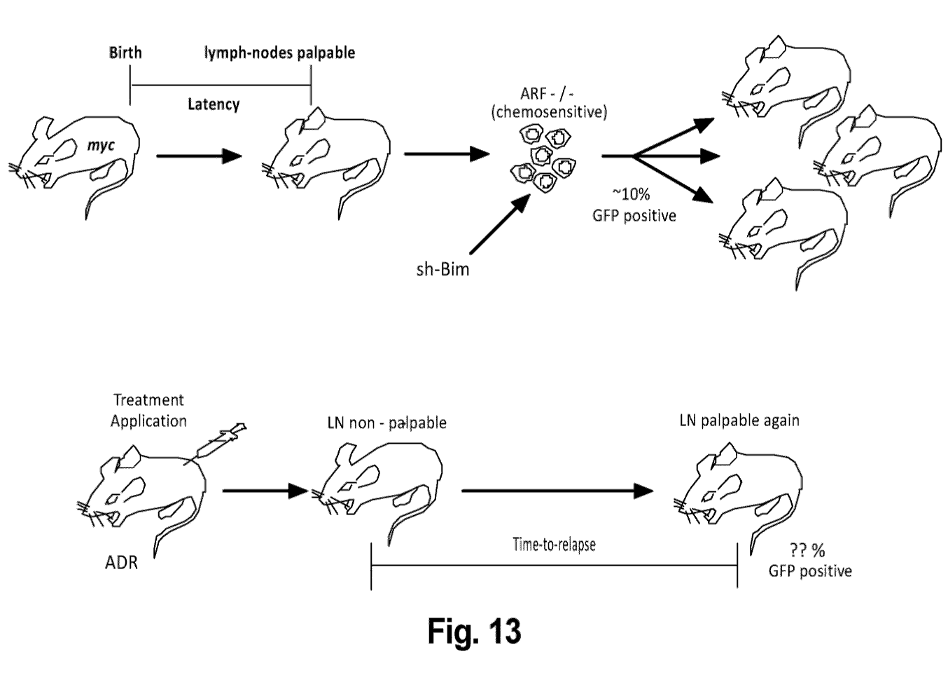

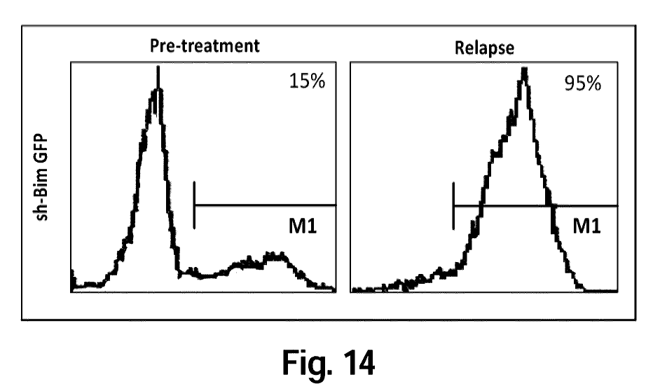

Given this ability of shRNAs to modulate tumor treatment response in tumors arising from shRNA-modified stem cells, we wanted to determine whether stable RNAi could modulate chemotherapeutic response acutely in mature tumors. Previous work from our group has shown that Em-Myc ARF−/− tumors are sensitive to adriamycin treatment (Schmitt et al, Cell 2002). To determine whether stable RNAi could alter the treatment response of chemosensitive tumors, we infected Em-Myc ARF−/− tumors with either a control vector or MSCV Puro-IRES-GFP Bim shRNA (Schmitt et al. Nature Med 2000). Following infection, the number of infected tumor cells was assayed by FACs analysis, and equal percentages of control and shBIM-infected tumors cells were injected into WT recipient animals (FIG. 13). Tumors arising in recipient animals were treated with 10 mg/kg adriamycin. Relapsed tumors were assessed for GFP content by FACs analysis (FIGS. 13 and 14). In the case of control-infected tumors, relapsing tumors were GFP-negative, suggesting that the presence of the vector conferred no selective advantage on these tumor cells. However, tumors relapsing after shBIM stable infection were invariably GFP-positive, indicating that the tumor cells expressing the Bim hairpin had a selective advantage after treatment. This data establishes that shRNAs can modulate tumor sensitivity, and that shRNAs can be used to screen for mediators of drug sensitivity.

These data demonstrate the feasibility of a a global strategy to identify modifiers of drug action in vivo. Specifically, if an shRNA is enriched during treatment responses (as occurs for shBIM), then inactivation of the target gene confers a survival advantage during treatment. As such, the nature of such shRNAs will provide insight into the molecular basis of drug action as well as to potential mechanisms of drug resistance. In contrast, if an shRNA is depleted, then inactivation of the target gene sensitizes the cell to killing in the presence of the drug. The nature of these depleted shRNAs will provide insights into possible targets or pathways that would work in combination with the drug. Of note, while studies may be performed on individual shRNAs, the development of ‘bar-coded’ shRNA libraries (described herein) will greatly facilitate this effort. Finally, while these experiments use mouse tumors, similar studies may be performed on human tumor cells in xenograft settings.

### Example 5

### SIN shRNA Vectors

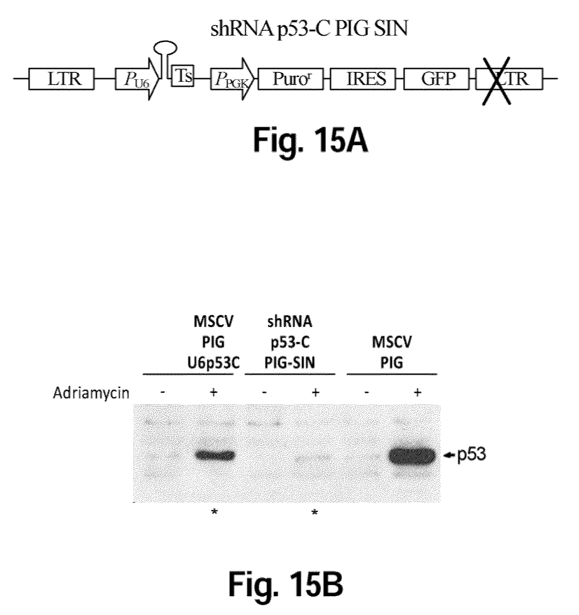

We have generated Self-INactivating retroviruses that express shRNAs. These viruses, based on the Clontech pQCXIX self-inactivating retrovirus contain an inactive 5′ LTR following viral insertion, resulting in the absence of long viral transcript expression. Experiments with p53 shRNAs (as described in Example 1) show that these vectors produce significantly better suppression of p53 in mouse embryonic fibroblasts than MSCV vectors expressing the same shRNA (FIGS. 15A and B). This provides the first direct evidence that the SIN vectors may be more effective than standard vectors.

### Example 6

### Characterization of Germline Transgenic Mice

As described above, Applicants have developed methods for generating mice expressing shRNAs in the germline. Applicants have further characterized p53 shRNA expressing mice generated using lentiviral transduction.

A lentiviral vector encoding our “p53C” shRNA was used to infect embryos and produce mice expressing a functional hairpin. Further characterization of these mice shows that of 10 pups born, 3 founder mice (#3, #8, and #10) were confirmed to harbor the shRNA construct by GFP fluorescence, PCR and Southern blot. Genomic DNA from each animal was digested with Pst 1, Southern blotted and hybridized with a GFP+WRE probe as per protocol in Lois et al. 2002. Southern blots of tail DNA indicate that each founder animal has have a single proviral insertion. This is important, as it will minimize complications associated with multiple gene copy numbers and providing a simple method of tracking transgenic animals.

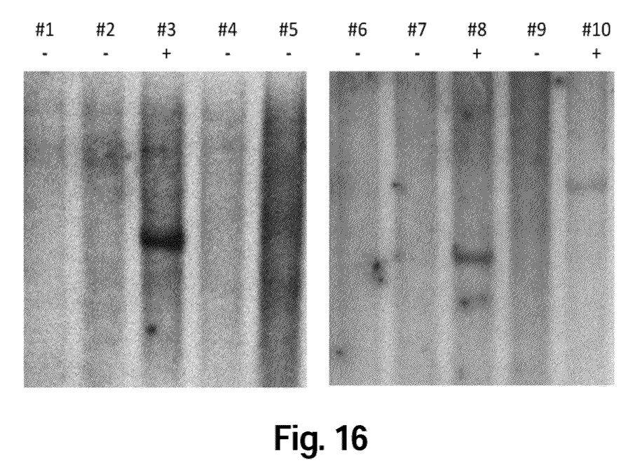

Western analysis of p53 in the dermal fibroblasts of the transgenic founder mice has revealed that p53 protein levels are significantly reduced, even in the presence of the DNA damaging agent adriamycin (FIG. 16). In contrast, the non-transgenic littermate controls (#1 and #2), as expected, show robust p53 activation in response to adriamycin treatment. Thus, we are able to achieve stable RNAi in the whole animal.

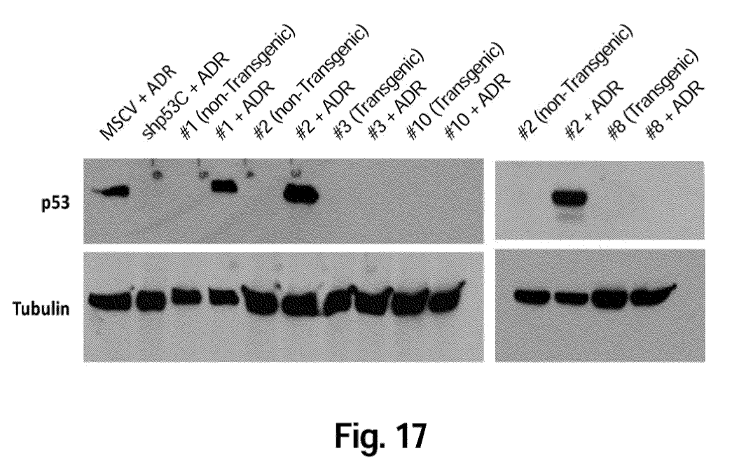

To confirm the functionality of the p53 hairpin, we performed colony-formation assays using the dermal fibroblasts isolated from the transgenic founders and non-transgenic littermates. In this assay, p53 deficiency results in a greatly enhanced ability of untransformed cells to form colonies when plated at clonogenic density. Data shown in FIG. 17 indicate the ability of the fibroblasts from the transgenic founder mice to form significantly more colonies compared to fibroblasts from the non-transgenic littermate controls. Consistently, cells from the non-transgenic animals underwent replicative senescence at approximately passage 7 (as assessed by growth rate, morphology, and Senescence-Associated β-galactosidase staining). In contrast, no senescent cells have been detected in cells obtained from the transgenic founders (currently at passage 12).

Finally, Applicants have demonstrated the ability of the founders to transmit the transgene to their progeny. Transgenic founder mouse #10 produced 2 separate litters of pups, several of which were positive for GFP and by PCR of regions of the vector.

### Example 7

### Generation of shRNA Libraries and Highly Parallel Screening

Applicants have constructed a partial genome-wide library of RNAi inducing constructs that will eventually target every gene in the human genome. Applicants have targeted ˜8,500 genes with approximately 23,000 sequence-verified shRNAs. Each is carried in a validated, MSCV-derived vector that is immediately useful for stable suppression. However, Applicants have also designed the vectors to have the capability of moving the inserts to other vectors via a recombination strategy that occurs in vivo following bacterial mating. Applicants can easily move any insert from the library into the lentiviral backbone that is used for transgenesis experiments described above.

Additionally, each component of the library is tagged with an individual barcode. These allow one to follow the changes in the numbers of cells representing individual clones in the library (in a mixed population) using oligonucleotide microarrays. Applicants have prepared such arrays and are now testing the possibility of doing large-scale synthetic lethality screens using this strategy.

In the one version of the library, the distribution of shRNAs was skewed to enrich for sequences that matched also the mouse homolog of a given gene. This has resulted in our accumulating about 6,000 mouse shRNA constructs so far. A second generation library is a specifically targeted mouse library. Applicants have selected approximately 1,200 genes, which have each been targeted with 5 shRNA sequences. Genes in this set were selected based upon their cancer relevance and were hand-curated.

Each shRNA expression cassette in the mouse and human RNAi libraries is associated with a unique 60 nucleotide barcode. This permits the use of population genetics as an approach to the search for both positively and negatively selected epigenetic lesions in screens of the libraries. For example, imagine a search for shRNAs that enhance the sensitivity of cells to doxorubicin or a targeted therapeutic. Cells would be infected with the library such that each of the 20,000 shRNAs is represented by 100-1000 infected cells. This population is treated with the drug at a relatively low concentration, e.g. EC10. By comparing untreated and treated populations, we might find shRNAs that enhance sensitivity to a low concentration of drug, since these would be selectively lost from the population. The ability to conduct such a screen depends upon parallel analysis of individual cell populations expressing shRNA constructs. One could also examine the behavior of pure homogeneous populations of cells bearing individual shRNAs in 96 or 384 well plates. However, the availability of barcoded vectors lets us track the frequency of individual shRNA clones in a mixed population, allowing highly parallel assays to be conducted in vitro or in vivo.

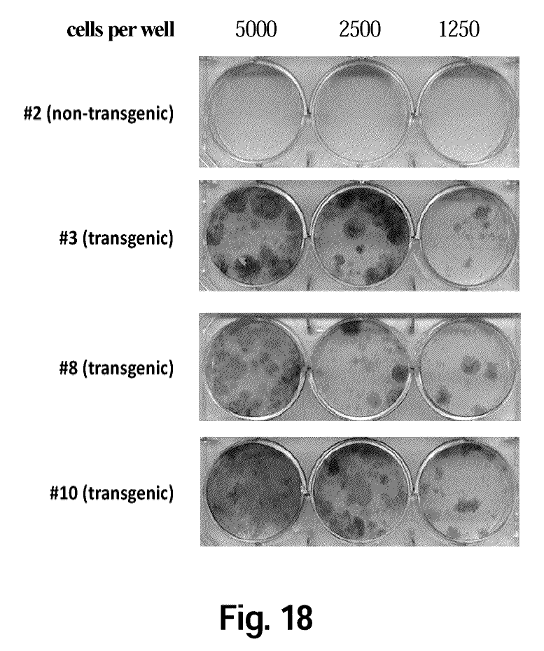

Barcode arrays corresponding to the 22,600 hairpins in the human shRNA library have been synthesized. These have been validated by self-self hybridizations using both DNA from the E. coli library and DNA where the barcodes have been amplified from the genomic DNA of library-infected 3T3 cells. Quality control test have demonstrated that the arrays perform well, with 2,600 negative controls appearing as negatives, and with the barcodes known to be represented in the population giving positive signals. There are a small number of false positives (<1%) that may be eliminated by further optimization of hybridization conditions. Examination of a comparative intensity plot shows most spots reporting consistently in Cy3 and Cy5 labeled material. All of the spots falling off of the diagonal can be accounted for by an easily recognizable anomaly in the hybridization signal (FIG. 18).

### Example 8

### Certain Transgenic Animal Protocols

a) ShRNA Transgenic Mice:

Isolation of shRNA ES-cell lines. Standard ES-cell techniques are employed. A 129S6/SvEvTac TC1 cell line was obtained from Harvard Medical School (Boston, Mass., Dr. P. Leder). The ES-cells are routinely maintained between passage 11-15 by culture on irradiated MEF-feeder cells in ES-media further supplemented with LIF-containing conditioned media. 20 μg of linearized plasmid DNA is electroporated into ˜107 ES-cells. The electroporated cells are plated onto gelatinized plates and cultured in ES-media supplemented with LIF-containing conditioned media. After two days Geneticin (Roche) is added to an active concentration of 300 μg/ml. The cells are cultured for an additional ten days to allow colony formation. From each selection ˜50 colonies with undifferentiated ES-cell morphology are cloned by trypsinization and 96-well plates. After 4 further days of growth the cells are cryopreserved in situ on two of the 96-well plates to preserve them at early passage. The third replicate cultures are then grown further by passage to 12-well then 6-well plates. At that point separate aliquots of cells are cryopreserved, and lysed for either DNA, RNA or protein isolation to determine transgene presence and knockdown of target gene expression.

Chimeric mouse production. Blastocysts are isolated from 8 super-ovulated E3.5d pregnant C57Bl/6 mice and cultured in ES-cell media. ES-cells are trypsinized to single-cells and washed in ES-media. Five to ten ES-cells are injected into each blastocysts. The injected ES-cells are then transferred to the uterus of 2.5d pseudo-pregnant CD-1 foster females in batches of 8-10. For each cell line 50 blastocysts are injected. Chimeric pups are born 17 days post-injection. The degree of ES-cell contribution in chimeric pups is estimated from the degree of agouti coat color. In our experience the TC1 cell line, although XY in karyotype, frequently generates gametes in both male and female chimeras. Thus 4-6 high percentage chimeras of either sex are bred to C57Bl/6 females to determine the degree of germline contribution of the ES-cells in each chimera through coat color genetics of the F1 pups. Germline-competent chimeras are then bred to 129/SvEvTac mice (from Taconic Farms) to maintain the shRNA transgene on an inbred background. The presence of the shRNA transgene in F1 pups is determined by PCR of tail biopsy DNA.

b) Lentiviral Transgenics

shRNA expressing lentiviruses are resuspended at 106 ifu/ml in M2 media, aliquoted in 10 μl portions and stored at −80 degrees. For sub-zonal injection of fertilized mouse eggs the viral suspension is thawed and centrifuged briefly in a table-top microcentrifuge. Five microliters of suspension is then placed under mineral oil on a glass coverslip mounted in an injection chamber. Also on the cover slip is placed a 5 μl drop of CZB medium supplemented with 1 μg/ml Cytochalasin B. Fertilized eggs are incubated for 10 minutes in the CZB-cytochalasin prior to injection. For injection the viral suspension is picked up into a micropipette with a 2-5 μM aperture. The injection pipet is transferred to drop with the eggs. Positive pressure of 0.5-2 PSI is applied to the viral suspension to promote a slow steady outward flow. Each egg is then picked up with a holding pipet and the injection pipet is allowed to puncture the zona pellucida of the egg. A slight swelling of the zona indicates flow of the viral suspension into the peri-vitteline space. Each egg is injected similarly. Following injection the eggs are transferred to a dish of M2 media and then sequentially through four 200 μl drops of M2 media to dilute the cytochalasin B. Finally the embryos are transferred to a 37 degree incubator for culture in M16 media. All of the injection pipets, injection chambers, etc are rinsed in 70% Ethanol:1% SDS to inactivate lentiviruses.

Injected embryos are transferred to the oviduct of pseudo-pregnant CD1 mice. Potentially transgenic pups are born 19 days later. At 1 week of age tail biopsies are performed for DNA extraction. The tail DNA is screened by PCR to identify transgenic pups with genomic lentiviral insertions. Positive pups will be further screened by southern blot DNA analysis to determine copy number of the insertions.

### Example 9

### Generation of Chimeric Mice Using RCAS/TVA

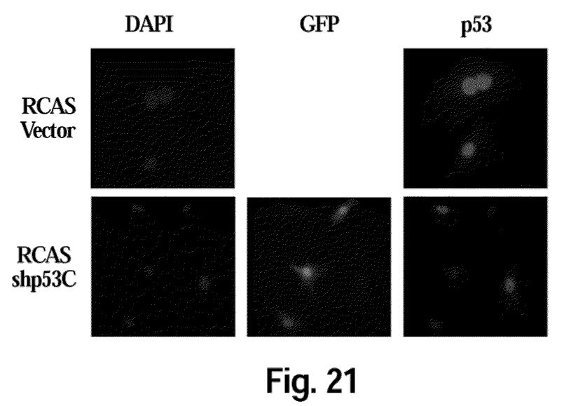

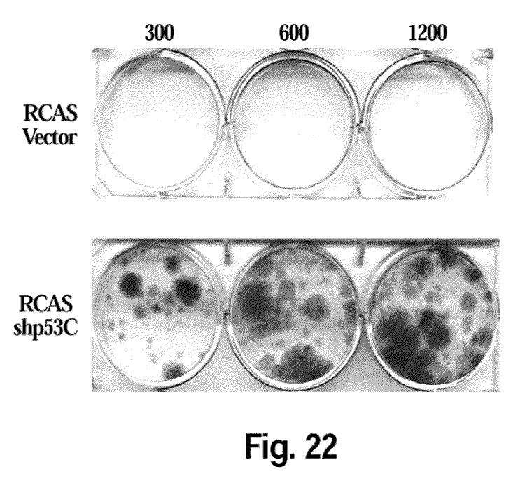

Applicants have generated a vector system that will allow tissue specific expression of shRNAs in vivo. This approach involves infecting cells expressing an avian viral receptor under the control of a ubiquitous or tissue-specific promoter in vivo. Applicants have modified the RCAS vectors to optimally express our RNAi haripins in mice and generated vectors that express shRNAs targeting mouse p53. As a proof of the system, Applicants generated virus from these constructs and used it to infect MEFs stably expressing the avian viral receptor. The functionality of these hairpins was confirmed by immunofluorescence, using p53 antibodies, which showed a dramatic reduction in p53 levels in cells infected with RCAS p53 shRNA constructs (infected cells are GFP-positive) (FIG. 21). This apparent loss of p53 was confirmed in a classic p53 functional assay. Specifically, MEFs infected with RCAS p53 grew well when plated at low density, while control cells were unable to produce colonies (FIG. 22). This data establishes that shRNAs can effectively target genes when expressed from RCAS retroviral vectors.

### Example 10

### Generation of ES Cells Expressing shRNAs

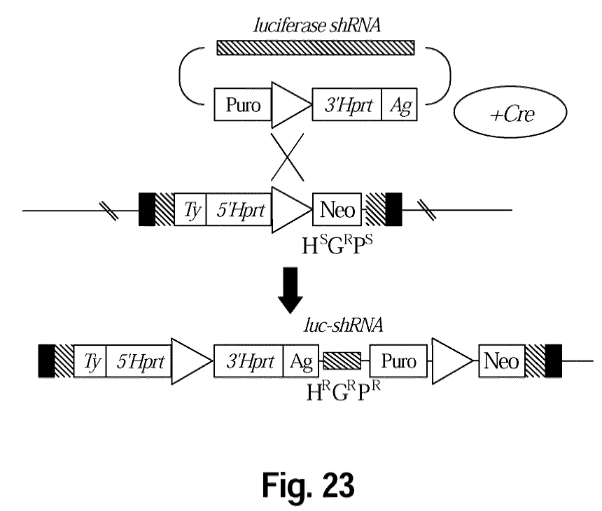

This examples describes a system for creating genetically defined RNAi “epi-alleles” in mice using Cre-mediated recombination to stably integrate a single RNAi expression cassette into a single locus in the mouse genome. This technique will minimize clonal variation due to random integration events seen in other studies and should allow for the efficient creation of “epi-allelic” series of RNAi constructs, as well as an inducible RNAi system. Applicants have adapted a system developed for chromosomal engineering in mice to mediate the integration of a single short hairpin RNA (shRNA) expression cassette in mouse ES cells. This strategy relies on the ability to integrate a “donor” plasmid, containing a shRNA expression construct, into an “acceptor” locus through the transient expression of Cre recombinase (FIG. 23). This system is designed so that proper recombinants can be selected for, through the reconstitution of the mini-HPRT gene and a drug resistance gene (eg, puromycin). Additionally, both the donor and acceptor constructs express coat color gene markers, either Agouti or Tyrosinase, which can be used to score chimeric mice.

This system has been tested in hprtΔ ES cells at the D4Mit190 locus. By co-transfecting either a Cre expression vector and the shRNA donor plasmid or the donor plasmid alone, 100% of HPRT reconstituted ES cell colonies (ie HATr colonies) (90 of 90) contain correctly integrated donor plasmids (as scored by genomic PCR). Importantly no HATr colonies were observed in the absence of Cre recombinase, suggesting that this scheme is highly effective at inducing site-specific integration in ES cells.

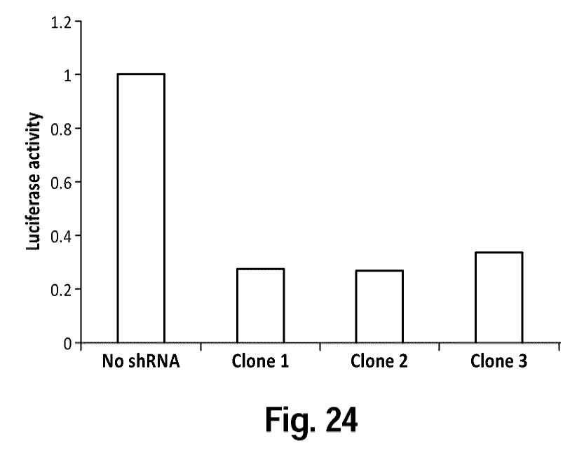

To test the effectiveness of this approach at evoking gene silencing in ES cells, Applicants integrated an shRNA cassette expressing a hairpin targeting Firefly luciferase. Individual HATr clones were isolated and transiently transfected with plasmids expressing Firefly luciferase (i.e., the target gene) and Renilla luciferase (i.e., a transfection control which is not targeted). The results, shown in FIG. 24, demonstrate that clones harboring the Firefly shRNA can potently suppress luciferase activity, (approximately 5-fold relative to control cells).

### Example 11

### Reversible RNAi In Vivo

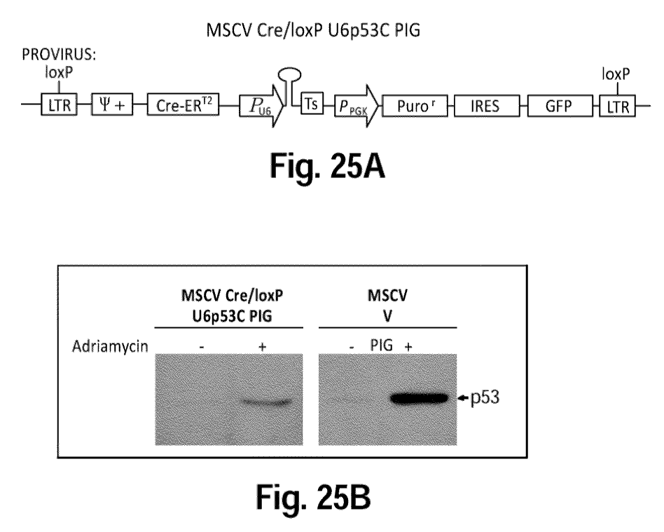

Applicants have generated a novel retroviral vector (MSCV CreER/loxP U6shRNA PIG; FIG. 25A) containing all the genetic components required to reversibly inhibit gene function by RNAi. This vector is based on the MSCV U6shRNA GFP vector (see above).

To facilitate conditional deletion of the provirus, a loxP site is engineered into the NheI restriction site of the MSCV 3′ LTR, resulting in a floxed provirus upon integration (FIG. 25A). In addition, Applicants placed a cassette encoding the CreER^{T2} fusion protein upstream of the U6shRNA cassette, under the control of the viral 5′ LTR promoter. In normal cells, CreER^{T2} is cytoplasmic and inactive, however addition of tamoxifen activates the recombinase activity of the fusion protein.

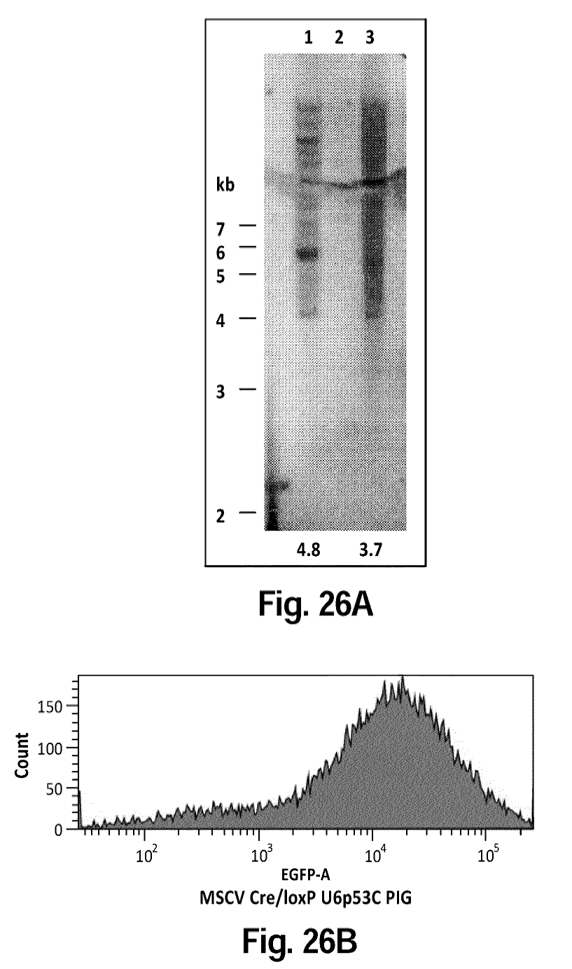

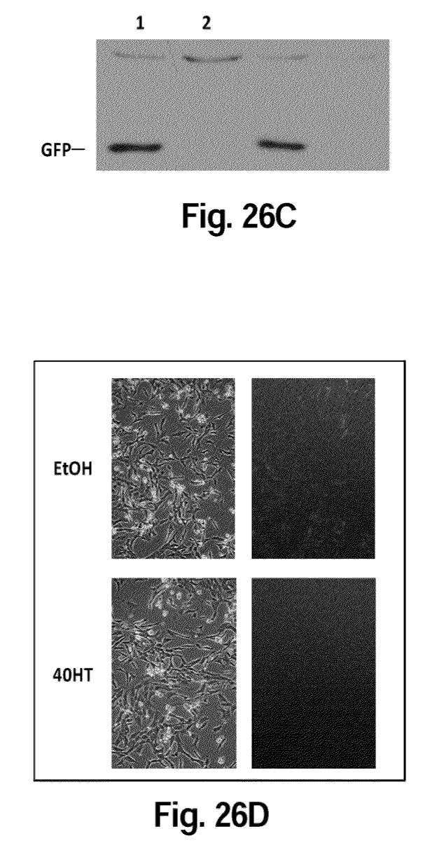

Using the p53C shRNA, Applicants have shown that each component of the vector appears to be functional. MEFs infected with MSCV CreER/loxP U6p53C PIG virus show stable suppression of p53 expression by Western blot (FIG. 25B). Therefore the CreER fusion protein and loxP sites do not interfere with shRNA production. Addition of 0.5 μM 4-hydroxytamoxifen (4OHT) to cultured cells infected with MSCV CreER/loxP U6p53C PIG virus results in deletion of the provirus from the genome, as measured by Southern blot using a probe that hybridizes to the GFP cassette in the provirus (FIG. 26A). As expected, 4OHT treatment and excision of the provirus also leads to loss of GFP expression, as measured by Western blot (FIG. 26B) or FACS (FIG. 26C). Fluorescence microscopy also shows loss of GFP flourescence upon 4OHT treatment of cultured cells infected with MSCV CreER/loxP U6p53C PIG virus. These results demonstrate that the CreER fusion protein encoded by the provirus can effectively excise the provirus itself. Importantly, 4OHT treatment does not appear to affect growth of uninfected cultured cells, and excision of the provirus occurs after only 24 hours of 4OHT treatment. This self-excising strategy has three major benefits: (1) the timing of Cre activation can be controlled; (2) long-term Cre toxicity is avoided; and (3) all infected cells (producing shRNAs) have the intrinsic potential to delete the provirus. Each of these factors are important when adapting this approach to in vivo tumor models.

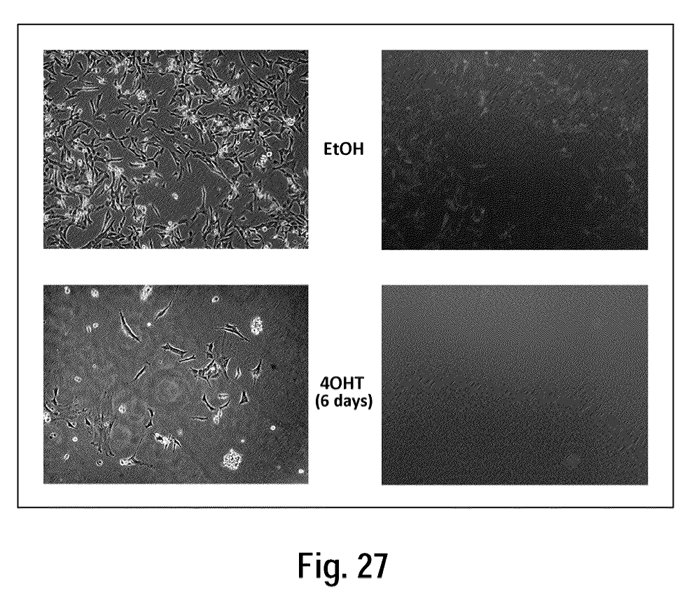

Applicants have examined the effects of reversing RNAi-mediated knockdown of p53 expression in cultured primary cells. Initial observations indicate that excision of the p53C shRNA-producing cassette in late passage murine embryonic fibroblasts causes substantial cell death (FIG. 27). Applicants have also initiated in vivo “reversible tumorigenesis” experiments using the Eμ-myc lymphoma model. Systemic tamoxifen treatment has proven effective in other animal model systems and it should be able to effectively reverse RNAi-mediated suppression of gene expression in established tumor cells in vivo. The MSCV CreER/loxP self-excising viral vector should allow us to test proof of principle for “hit and run” gene therapy approaches based on RNAi or gene overexpression.

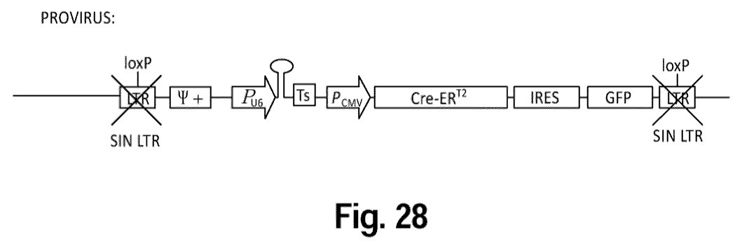

A second generation vector is shown in FIG. 28. This vector has several modifications that may make it more effective. First, the retroviral vector is a contains a self-inactiating (SIN) LTR such that, upon provirus integration, there is no transcription from the 5′ LTR. This modification should increase the effectiveness of shRNA mediated silencing, as shown in ‘RNAi stem cells 1; FIG. 28. Second, the cre-ER IRES GFP cassette is placed downstream of the strong CMV promoter, which will increase the expression of both components, allowing better excision of the provirus upon tamoxifen addition and better visualization of GFP in vitro and in vivo. Note also that other recombination systems and regulatable recombinases could be used as well.

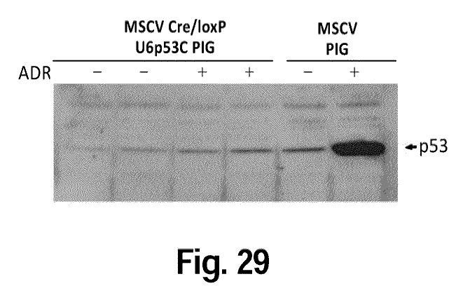

This vector or similar ones (e.g. based on lentivirus technology) will have broad applications for in vitro and in vivo use. First, one can envision manipulating stem cells ex vivo with an shRNA in a reversible way (i.e. ‘hit and run’ gene therapy). This might be advantageous in settings where transient gene suppression is desirable or, in the event that some hairpins direct stable gene silencing (as can occur in some species), removal of the vector leaving the suppression intact. In fact results indicate that excision of a p53 targeted shRNA construct from a cell does not result in recovery of p53 expression (FIG. 29). This indicates that an epigenetic change is occurring, resulting in a permanent or at least heritable inhibition of p53 expression even in the absence of a shRNA construct. Cells may therefore be transfected with a shRNA construct ex vivo to initiate downregulation, the construct removed, and the cells administered to a patient. In this manner, a patient receives genetically unmodified cells that have an engineered gene expression pattern. Second, for the construction of animal models of human disease, one envisions inactivating a gene using an excisable shRNA, allowing a phenotype to be produced, and then reversing the mutations to see whether the phenotype is rescued.

One example would be to inactivate a tumor suppressor gene, allow a cancer to form in an animal, add tamoxifen to excise the provirus (and shRNA) and then determine whether the cancer progresses upon re-expression of the tumor suppressor. This will show whether the tumor suppressor gene is required for tumor maintenance of the tumor, and would determine whether the pathway might be suitable for therapeutic intervention (i.e. if the tumor suppressor is required for tumor maintenance the pathway would be a good target). A second, broader, application would be to generate animal models of recessive human disorders using ES cells or some other stem cell type. Upon the appearance of a deleterious phenotype, tamoxifen can be administered to the animal, which is subsequently monitored for reversal of the deleterious phenotype. For example, one could produce a mouse model of muscular dystrophy or a neurodegerative disease by suppressing the causative gene, and then ask, at what point during the progression of the disease, the phenotype is reversible (in some settings the disease may have progressed beyond a point of no return). Such information would provide a guide as to when a disease can be corrected by pharmaceutical means or gene therapy.

### Example 12

### Conditional Regulation of Target (Tumor Suppressor) Genes In Vivo Using RNAi

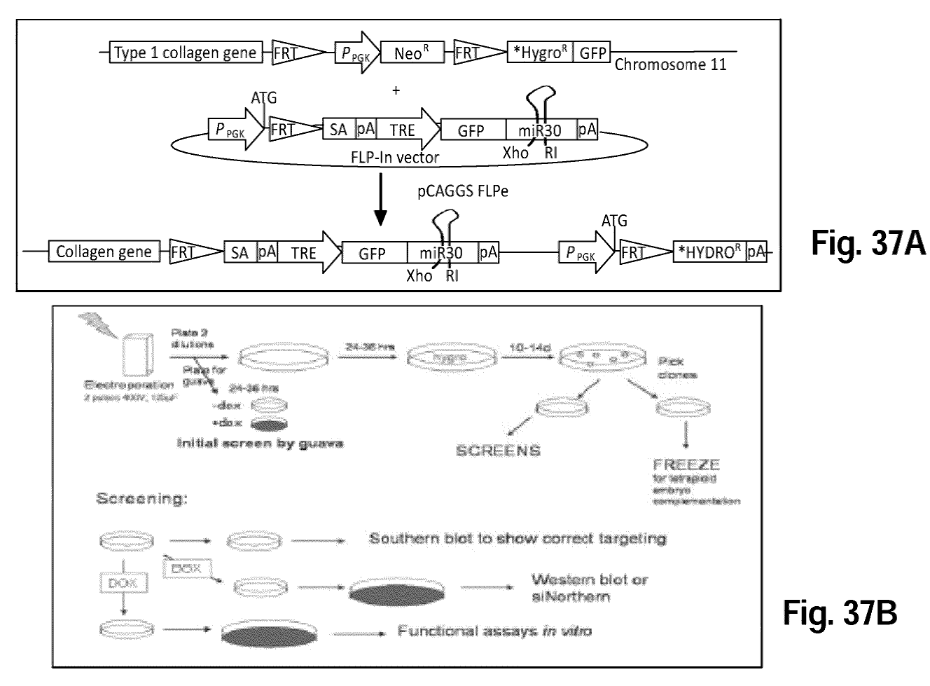

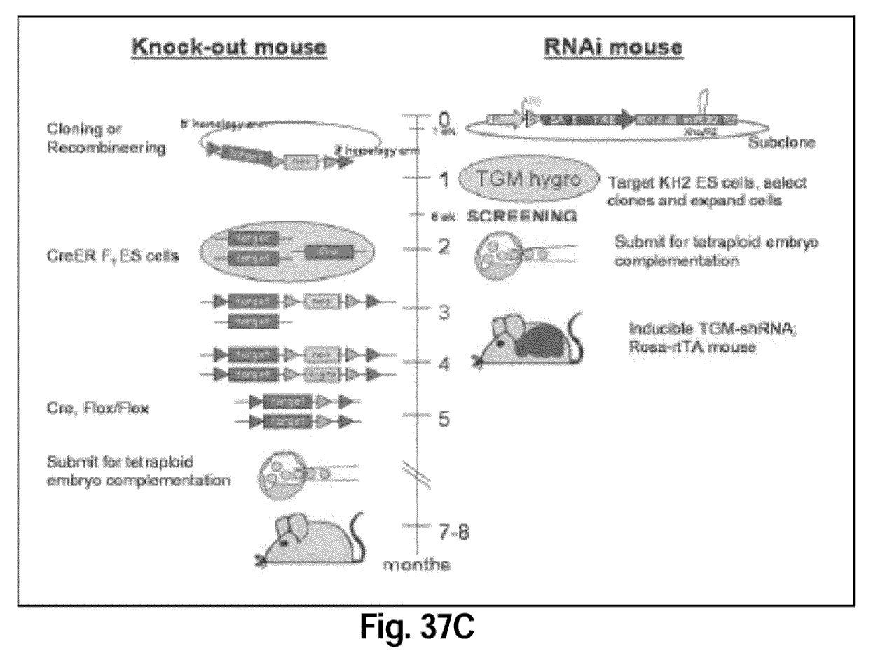

Applicants have shown that RNAi can cause sufficient gene expression “knockdown” to recapitulate the phenotypes of gene knockout in animals, particularly in cancer models. Unlike traditional knockout experiments, RNAi knockdown has the powerful advantage of reversibility, since the endogenous gene remains intact, and wild-type gene expression may be restored at select temporal and/or spacial patterns using the subject systems. In addition, RNAi knockdown experiments typically take about half as long as the time required to conduct traditional knock-out mouse experiments (see FIG. 37).

Certain examples described herein above provide reversible RNAi animal models to demonstrate the use of RNAi to spatially, temporally and reversibly regulate gene expression of any endogenous gene of interest. However, in certain models generated by classical transgenesis using standard pronuclear injection (see Example 1), there is little control over the integration site and copy number of the RNAi transgene. One drawback for this type of animal model is that numerous animals may need to be screened in order to identify founders with the desired robust expression of RNAi constructs (such as short hairpin RNA or shRNA, or miRNA constructs). Furthermore, various tissues may need to be tested due to the potential variability of expression in different tissues of each founder. These drawbacks can be circumvented by using genetically defined RNAi “epi-alleles,” such as the system described in Example 10 (Cre-mediated recombination to stably integrate a single RNAi expression cassette into a single locus in the mouse genome). This technique minimizes colonal variation due to random integration events.

Applicants hereby describe an additional example of targeted integration of RNAi constructs using the FLP/FRT recombinase system targeting the ColA1 locus in engineered embryonic stem (ES) cells (Beard et al., Genesis 44: 23-28, 2006, incorporated herein by reference). In brief, a tet-regulated microRNA (miRNA) is placed immediately downstream of the endogenous ColA1 gene in ES cells, which are subsequently used to generate ES cell-derived mice by, for example, tetraploid embryo complementation or blastocyst injection.

With the systems described herein, Applicants are able to rapidly generate numerous conditional, reversible, and/or tissue-specific gene knockdown animal models, thus providing powerful tools to dissect the function of any endogenous gene in vivo. This may be especially useful for cancer research, where reversible knockdown allows one to study the roles of certain essential genes (such as tumor suppressor genes) on tumor maintenance, and determine whether their sustained inactivation is required for tumor survival and progression. Results from these studies would have enormous implications for drug target identification or validation and cancer therapy in general.

Although the system and methods described herein can be used for any gene in an organism, including endogenous gene or certain virally transmitted genes, the following example uses specific tumor suppressor genes as target genes for illustration. It should be understood that the systems and methods of the invention are not limited to particular types of target genes.

The following illustrative example relates to the effects of reestablishing the INK4a/ARF locus, which is one of the most frequent sites of genetic loss in human malignancies. Importantly, INK4a and ARF are silenced in cancers through promoter methylation, which makes these genes very applicable for pharmacological strategies of reestablishment of gene expression (by using, for example, DNMT/HDAC inhibitors).

One desirable feature of the invention is the ability to spatially and temporally control knockdown of nearly any endogenous gene in vivo. Applicants have adapted the tetracycline (tet)-regulated system to control expression of RNAi constructs from tetracycline-responsive promoters (TRE) (Dickins, Hemann et al. 2005). Briefly, the tet-based system requires the additional expression of a tet-transactivator protein (tTA or rtTA) (Furth, St. Onge et al. 1994). In the presence of tTA (tet-off), TRE driven expression is active, but is shutdown once doxycycline (a tetracycline derivative) is administered. The reverse is true for rtTA (tet-on), where transcription is active only in the presence of doxycycline. In mice, tTA or rtTA expression can be limited with use of tissue-specific promoters, making it possible to restrict knockdown to particular tissues.

For this study, RNAi technology will be used to generate regulatable gene knockdown in mice, specifically targeting knockdown of tumor suppressor genes (TSGs) to investigate their roles in tumor maintenance.

### Oncogene Dependence and Tumor Suppressor Hypersensitivity

Transgenic mouse models that conditionally express oncogenes have transformed our knowledge of the roles of oncogenes and their importance in tumor induction and maintenance. Subsequent to numerous observations, the idea of oncogene dependence emerged, where it is presumed that tumors become “addicted” to the overexpression of an oncogene and cannot continue to proliferate once the oncogenic stimuli is removed (Weinstein 2002; Jonkers and Berns 2004). Many studies support this concept and show that removal of the primary oncogenic stimulus following tumor formation causes regression in many types of cancers (Felsher and Bishop 1999; Chin, Tam et al. 1999; Fisher, Wellen et al. 2001). A recent study showed that repression of the c-myc oncogene induced cellular senescence in diverse tumor types including lymphoma, osteosarcoma, and hepatocellular carcinoma (Wu, Riggelen et al. 2007). However, this is not the case for all cancers. Osteosarcomas engineered to be deficient in p16INK4a or Rb failed to regress after myc inactivation (Wu et al. 2007). Similar results were also seen in a breast cancer model (Boxer, Jang et al. 2004). In fact, Boxer and colleagues describe regression in only 50% of tumors following repression of a myc transgene, implying that myc driven mammary tumors are less dependent on the driving oncogene. In this cancer model, the cells recovered their malignant properties and were able to generate more aggressive tumors independent of myc re-expression. This work underscores the progressive nature of cancer and the ability of tumor cells to rapidly acquire new spontaneous genetic lesions, allowing them to escape oncogene dependence. It is speculated that myc overexpression in tumorigenic cells permits sufficient cell expansion to allow collaborating mutations to take place.

A parallel concept to oncogene dependence is the idea of “tumor suppressor hypersensitivity.” Overexpression of a wild-type TSG (encoding, for example, p53, Rb, or APC) in human cancer cells lacking that TSG caused marked inhibition of growth, induction of apoptosis, and/or inhibition of tumorigenesis in mice even in the presence of an oncogene (Weinstein 2000). Surprisingly, correction of just one lesion can have growth-inhibitory effects in cancer cells that contain multiple mutations, suggesting the possibility of a “hypersensitivity” to specific tumor suppressor genes (Weinstein 2002). However, enforced overexpression of lost tumor suppressor genes may be nonphysiological regarding later therapeutic applications. The use of conditional RNAi, however, allows us to study the effects of restoration of endogenous expression levels of tumor suppressor genes downregulated in tumors. Applicants have successfully demonstrated this idea by showing that reactivation of endogenous levels of p53 in either transplanted subcutaneous tumors or liver carcinomas derived from H-ras/p53-knockdown MEFS or hepatocytes, respectively, was sufficient to cause long lasting tumor regressions (Dickins et al. 2005, 2007; Xue et al. 2007). Regarding later therapeutic applications, it is of particular interest that even short term pulse reactivation of endogenous p53 was sufficient to induce complete and long lasting tumor remissions. The reversibility was achieved using a TRE-promoter to drive expression of a shRNAmir targeting knockdown of p53 (sh-p53.1224). Upon administration of doxycycline, p53.1224 shRNAmir expression was repressed and p53 reactivation caused the tumors to regress. These studies demonstrate that in specific genetic contexts, continual loss of TSGs is required in order to maintain established tumors, highlighting the importance for further studies which could identify molecular targets for cancer therapy.

In contrast to amplified oncogenes, which can be direct targets for small molecule or antibody inhibitors, the therapeutic strategies for altered tumor suppressor genes are different. In some cases, deleted tumor suppressor genes directly pinpoint targets (e.g., PTEN→PIK3CA or Arf→MDM2) which can then be targeted. Candidates that are downregulated by promoter hypermethylation (e.g., Ink4A/Arf) can be targeted by demethylases and histone deacetylase (DNMT/HDAC) inhibitors. Tumor suppressor genes whose expression is mainly down-regulated due to an increased protein turnover can be accessible by specific inhibition of components of the proteasome.

Our system of shRNAmir transgenic mice holds great promise to help focus and expedite the drug development process, as functional in vivo validation increases the confidence in pursuing cost- and time-intensive therapeutic developments.

The Role of the INK4a/ARF Locus in Mouse Cancer Models

In recent years the INK4a/ARF locus been recognized as one of the most important players in cancer. In fact, loss of heterozygosity (LOH) or allelic imbalance at the INK4a/ARF locus is one of the most common abnormalities seen in a variety of human malignancies (For reviews, see Serrano 2000; Sharpless 2005; Sherr 2001). While numerous studies have characterized its role in tumor development, there is still little known as to its importance in tumor maintenance. Its role as a tumor suppressor locus first became apparent from murine tumorigenesis studies, which mapped carcinogen susceptibility alleles in BALB/c mice to the INK4a/ARF locus. This locus later became known as a major player in anti-tumor defenses, encoding for two proteins, p16INK4a (p16) and p19ARF (p19 or p14ARF in humans) through alternative open reading frames using different 5′ regulatory regions. These two proteins have non-overlapping functions and regulate distinct tumor suppressor pathways in response to aberrant growth or oncogenic stress, including ras and myc signaling (Kim and Sharpless 2006). Specifically, the INK4 proteins, including p16, are potent inhibitors of the cell cycle. They function by binding to cyclin-dependent kinases 4 and 6 (cdk4/6) and inhibiting their kinase activity. Subsequently, they activate the Rb pathway, leading to repression of E2F transcription factor activity and growth arrest. p19 also plays a role in tumor suppression, but it does so by binding and inhibiting MDM2, which normally ubiquitinates p53 and targets it for degradation. As a result, expression of p19 indirectly activates p53, a key protein that can initiate apoptosis and/or cause cellular senescence, processes that are important barriers to tumorigenesis (Hanahan and Weinberg 2000; Vogelstein and Kinzler, 2004).

The most common spontaneous tumors seen in INK4a/ARF-null mice are histiocytic lymphomas, consisting of mature B-cell and T-cell non-Hodgkins lymphomas (Sharpless 2005). This type of spontaneous tumor is not seen in p19ARF-null mice but comprises nearly 90% of all spontaneous tumors seen in INK4a/ARF-null animals, suggesting that combined loss of p16 and p19 enhances this tumor type (Sharpless 2005). In addition, a previous study clearly demonstrates that loss of the INK4a/ARF locus accelerates Eμ-myc induced lymphomagenesis similarly to loss of p53, and this loss can impact anticancer therapy by compromising p53 function (Schmitt, McCurrach et al. 1999). In this model, transgenic expression of the myc oncoprotein is driven from the immunoglobulin heavy chain enhancer, leading to a splurge of clonal B cell lineage lymphomas that closely resemble non-Hodgkin lymphoma in humans (Adams, Harris et al. 1985). Almost invariably in Eμ-myc lymphomas, INK4a/ARF or Trp53 is inactivated sporadically, confirming their dependence for the acquisition of a secondary cooperating mutation. This was shown in a study in which Eμ-myc transgenic mice were crossed to INK4a/ARF^{+/-} mice, and all Eμ-myc;INK4a/ARF^{+/-} succumbed to disease with a median tumor onset of 38 days compared to 130 days for control (Schmitt et al. 1999). Surprisingly, 88% of these mice lost the second INK4a/ARF allele and were thus functionally INK4a/ARF-null, causing severe impairment of apoptosis.

The study seeks to determine if reestablishment of the INK4a/ARF locus could promote tumor regression by restoration of apoptotic or senescence pathways in the presence of myc overexpression.

The INK4a/ARF locus has also been recognized to play an important role in breast cancer. In fact, loss of INK4a/ARF is one of the most common abnormalities, seen in nearly 58% of primary human breast cancers (D'Amico, Wu et al. 2003). More recent studies hypothesize that inactivation of p16 may be a very early event in breast tumorigenesis that occurs prior to any histological changes within the tissue (Zhang, Pickering et al. 2006). Also, decreased expression of p14ARF by promoter hypermethylation or deletion has been clearly linked with human breast cancer (Hanahan and Weinberg 2000). In many cases, the entire INK4a/ARF locus is affected, causing loss of both p16 and p14ARF and this often correlates with poor prognosis (D'Amico et al. 2003; Calvano, Rush et al. 1997; Serrano 2000; Kim and Sharpless 2000).

Clearly, INK4a and ARF are important players that cooperate with myc to produce more aggressive tumors; however, their roles in tumor maintenance have not yet been defined. Our model provides the necessary tool to address the therapeutic applicability of restoration of endogenous levels of p16 and p19 in these cancer models.

### Results

1. TRE-GFP-miR30 (TGM) Vector Design: An Enhanced TET-Regulatable shRNA Expression Vector

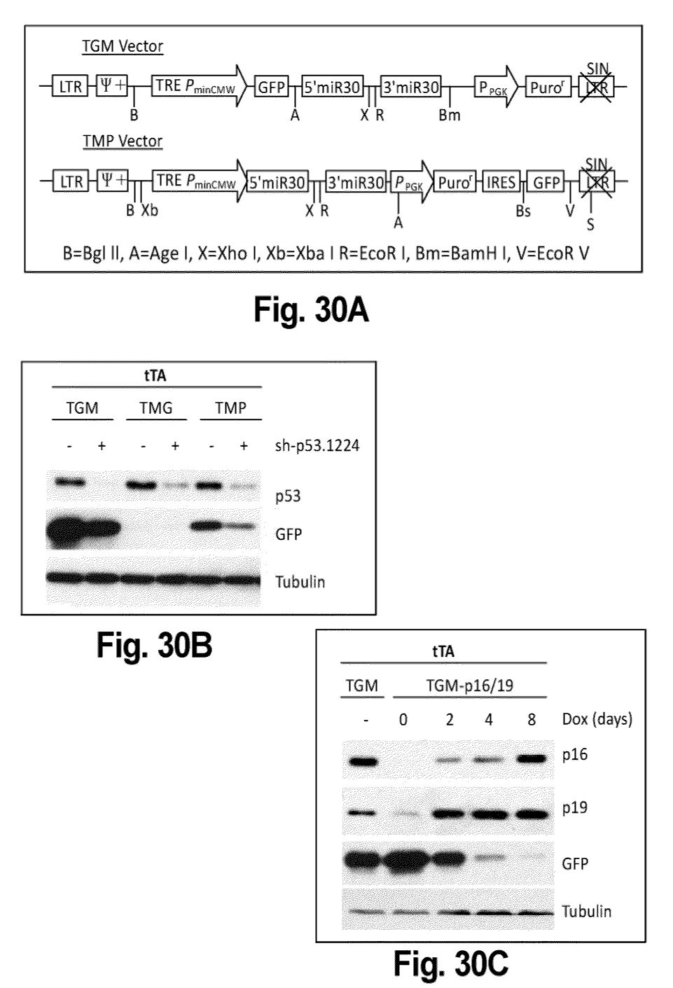

To develop mice with the most effective knockdown, Applicants designed a vector to include a spacer element (such as GFP cDNA) between the TRE and the mir-30 cassette (e.g., the TRE-GFP-miR30=TGM vector). Such a spacer region (e.g., coding sequences for a marker such as GFP, dsRed, or Neo^{R}) between the promoter and the RNAi cassett may promote the encoded miRNA function, and may further enhance target gene knockdown. Preliminary results show, using retroviral infection, that p53 knockdown is in fact more potent with sh-p53.1224 in the TGM vector compared to the TMP vector (FIGS. 30a, 30b). Similar results were seen with TGM-p16/19.478 compared to TMP-p16/19.478 (data not shown). TGM-p16/19.478 also confers reversible knockdown of p16 and p19 after doxycycline (Dox) treatment, where expression of both proteins return to control levels within 8 days (FIG. 30c). The TGM vector also provided an advantage due to the regulated GFP expression. Upon doxycycline treatment, GFP expression is shut down concurrently with miR30-shRNA upon doxycycline treatment; thus it is a useful visual marker of miR30-shRNA expression and will serve as valuable tool for rapid screening of transgenic founder lines.

In certain embodiments, the spacer region may contain any sequences, including non-coding sequences, coding sequences for an epitope or antigen, any fluorescent protein or enzymes (e.g., luciferase, AP, beta-galactosidase, etc.), drug resistance gene, or any other marker.

In certain embodiments, the RNAi cassette region may include any RNAi constructs, such as shRNA, microRNA based constructs, etc.

In certain embodiments, the TRE region may be replaced by any other art-recognized inducible/repressable expression regulatory sequences, such as lac operon, etc.

Alternatively, the spacer region may be absent (see the TMP vector design). Any markers encoded by the spacer region in the TGM vectors may be moved to other parts of the vector, such that the RNAi cassette is directly under the control of the promoter (such as the TRE-miniCMV promoter). IRES may be used to co-express two genes (such as drug resistant marker and a fluorescent protein, or any other combinations of different markers) under the same transcriptional control of a promoter (such as the PGK promoter).

In certain embodiments, SIN LTR may be used to enhance RNAi expression (Example 5).

In certain embodiments, a splice acceptor (SA) and a polyadenylation sequence may be placed upstream of the minimal tetracycline-responsive promoter (TRE) to reduce tetracycline-independent transcription by blocking readthrough from potential upstream promoters.

The FLPe recombinase can be provided from a separate vector, such as a vector that expresses the FLPe recombinase from the highly expressed CAGGS promoter (Buchholz et al., Nat Biotechnol 16: 657-662, 1998, incorporated herein by reference).

2. Improving Reversible Gene Knockdown in Mice

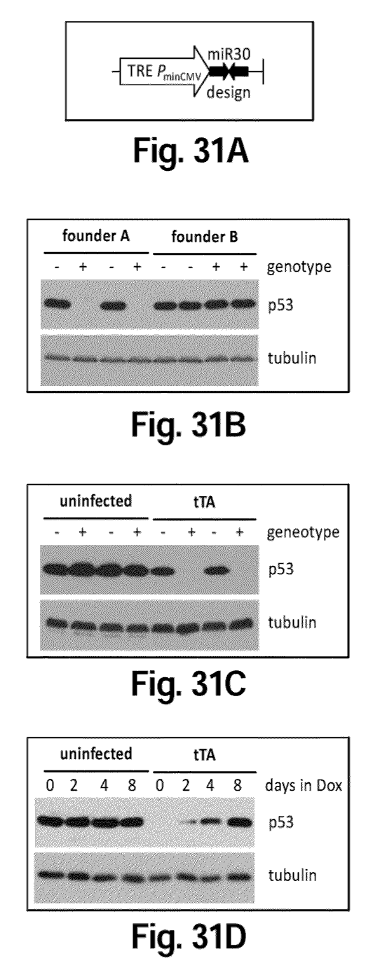

Some of our previous reversible mouse models were generated by standard pronuclear injection of a simple TRE-miR30 construct (e.g., TRE-p53.1224) isolated from the TMP vector (FIGS. 30a, 31a; Dickins et al. 2007). FIG. 31b demonstrates the variable expression of miR-shRNA between founder lines, where both Founder A and B contain the transgene but only Founder A exhibits robust knockdown of p53. This effect is likely due to the integration site of the transgene, in which Founder B may have insertion in a heterochromatically silenced region of the genome. This possibility necessitates screening multiple founders to identify a functional model. Regardless, a functional founder was identified, and primary mouse embryonic fibroblasts (MEFS) derived from TRE-p53.1224 transgenic embryos display potent knockdown of p53 when co-expressing the tetracycline transactivator protein (tTA) (FIG. 31c). Furthermore, p53 knockdown is reversible and tightly regulated by administration of doxycycline (FIG. 31d).

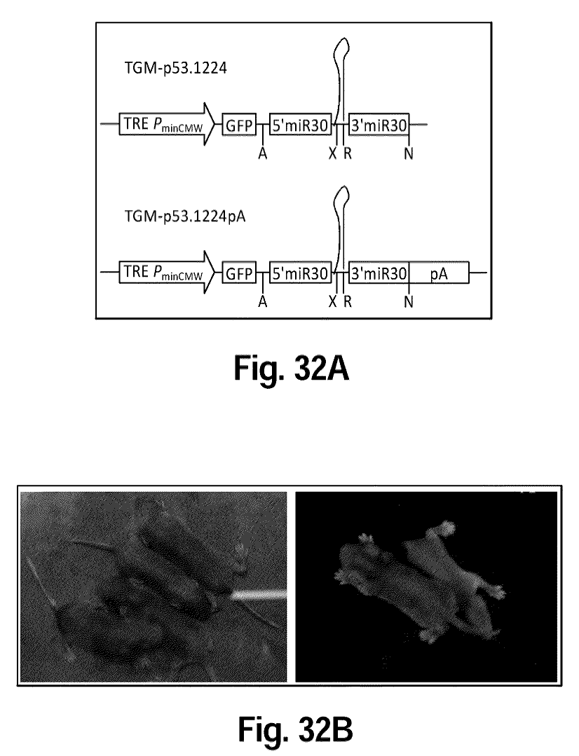

While standard pronuclear injection may require laborious and time-consuming screening protocols to identify functional founders, the approach is simple and allows rapid generation of several founder lines in which to test reversible knockdown. Under the presumption that using GFP to visualize miR-shRNA expression would expedite and simplify screening of founder mice, we generated a second generation of mice by pronuclear injection of a TRE-GFP-miR30 fragment isolated from the TGM vector. It was unclear at the time whether a polyA-tail was necessary for proper GFP expression, so two TGM constructs containing the p53.1224 shRNA, one with a polyA-tail and the other without (FIG. 32a), were used to generate mice. The founder mice were subsequently crossed to CMV-rtTA mice and progeny were initially screened for GFP expression in the skin (FIG. 32b). The double transgenic mice were also harvested for various tissues, and GFP expression was determined by western blot (data not shown). More clearly, these data illustrated the variability between transgenic lines and demonstrated the limitations of transgenesis, despite the apparent usefulness of the GFP marker as a powerful screening tool.

Following these preliminary results, Applicants improved the RNAi mouse technology by utilizing a site-specific targeting approach (similar to that described in Example 10) to incorporate the TGM construct in embryonic stem (ES) cells (see Methods). Site-specific integration of a single copy RNAi construct at certain locus (such as the Rosa26 locus, Siebler et al. 2007) can produce potent reversible knockdown.

3. shRNAs Targeting Knockdown of p16INK4a and p19ARF

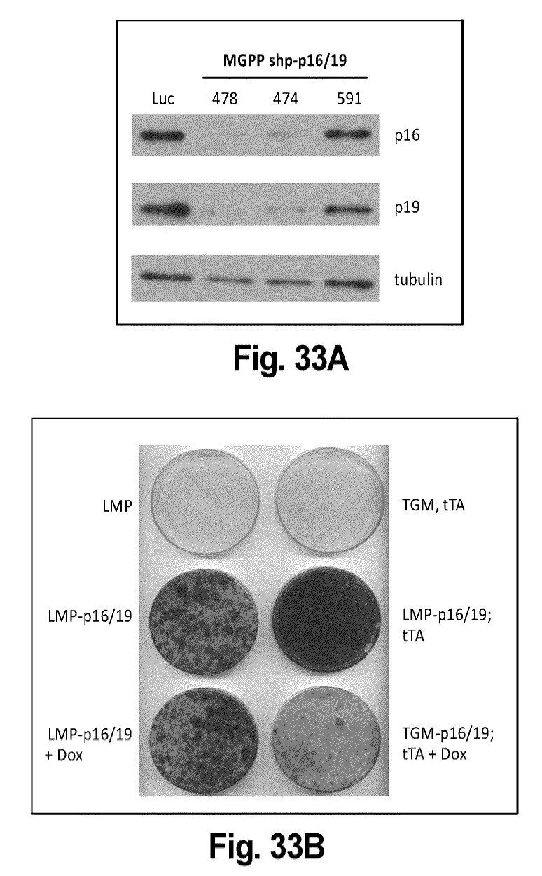

Before generating mice with single-copy shRNAs, Applicants first tested the shRNAs at single copy in vitro. Three shRNAs targeting exon 2 of the common p16 and p19 mRNA transcript were tested in a constitutively expressing retroviral vector similar to TGM at low MOI to ensure single copy integration in MEFs. Both sh-p16/19.474 and sh-p16/19.478 significantly knocked down both p16 and p19 levels when compared to control (FIG. 33a). sh-p16/19.478 was then cloned into the TGM vector for further analysis in vitro. In the presence of tTA (tet-off), doxycycline treatment shut off shRNA production and p16 and p19 levels were restored (FIG. 30c). When plated at low density, sh-p16/19.478 promoted colony formation, most likely attributable to the knockdown of p19 (FIG. 33b). This shRNA was used for the development of site-specific targeted mice (see Methods below).

### Experimental Design and Methods

1: Generating an Efficient and Reproducible Method for Achieving Reversible Gene Knockdown in Mice

As described above, shRNAmir transgenesis based on pronuclear injection resulted in high variability of knockdown efficiency between different founder lines. In order to resurrect the generation of shRNAmir transgenic mice, Applicants established site-specific integration of tet-promoter shRNAmir expression cassettes (TGM) into ES cells. Applicants used a system in which transgenes are targeted to the collagen 1 (ColA1) locus on mouse chromosome 11, a region that has been shown to be accessible for gene expression in most tissues (Beard et al. 2006). FLPe-mediated recombination is used to direct site-specific integration of the targeting vector into the ColA1 locus of KH2 mouse embryonic stem (ES) cells, which have been previously targeted by homologous recombination to introduce an frt-hygro-pA “homing” cassette downstream of the ColA1 locus. The FRT site at this locus confers high efficiency targeting of a transgene of interest (the TGM cassette in this case). Furthermore, this system allows for rapid screening by GFP expression because KH2 ES cells have been previously engineered to express rtTA under the control of the endogenous Rosa26 promoter. Correctly targeted ES cells can be quickly identified by GFP expression upon doxycycline treatment.

ES cells correctly targeted with the TGM vector were used to generate ES-cell derived mice by tetraploid embryo complementation (Schuster-Gossler, Lee et al 2001; Siebler, Zevnik et al. 2003, incorporated herein by reference). Tetraploid embryo complementation provides the advantage that founder mice should be completely transgenic and transmit the transgene through the germline to all progeny, which accelerates the process of establishing a colony by many months. Using the established protocol, Applicants generated and characterized a functional shRNAmir transgenic mouse (ColA1-TGMp16/19.478). The data suggests that the method of site-specific integration overcomes the problem of reproducibility and control for random integration, multiple copy number, and potentially eliminates silencing effects that are often seen in transgenic mice produced by traditional pronuclear injection. In addition, the ColA1 locus has been reported to be accessible for gene expression in many tissues including the spleen, thymus, lymph node, liver, intestine, pancreas, and skin, thus it is likely that these tissues may have sufficient shRNAmir expression necessary to produce knockdown of specific targets.

Methods:

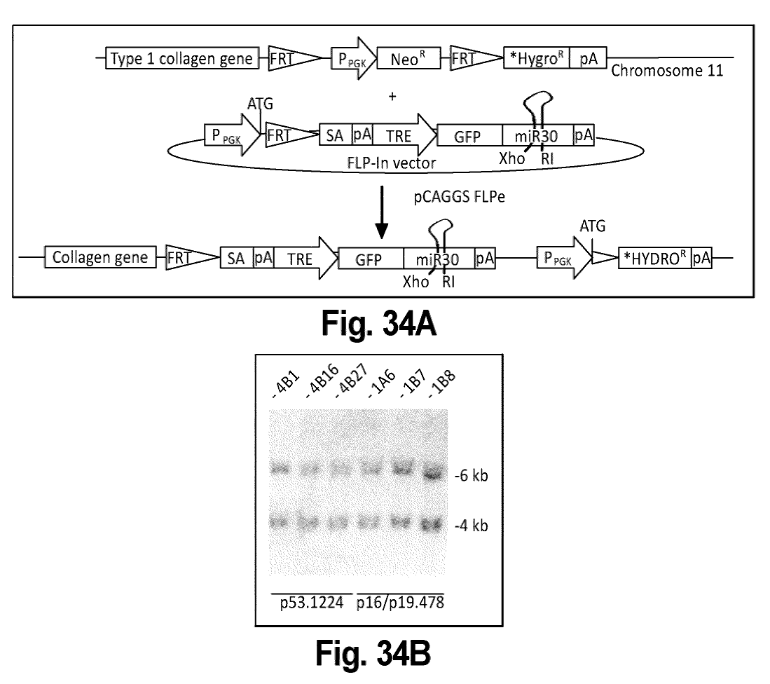

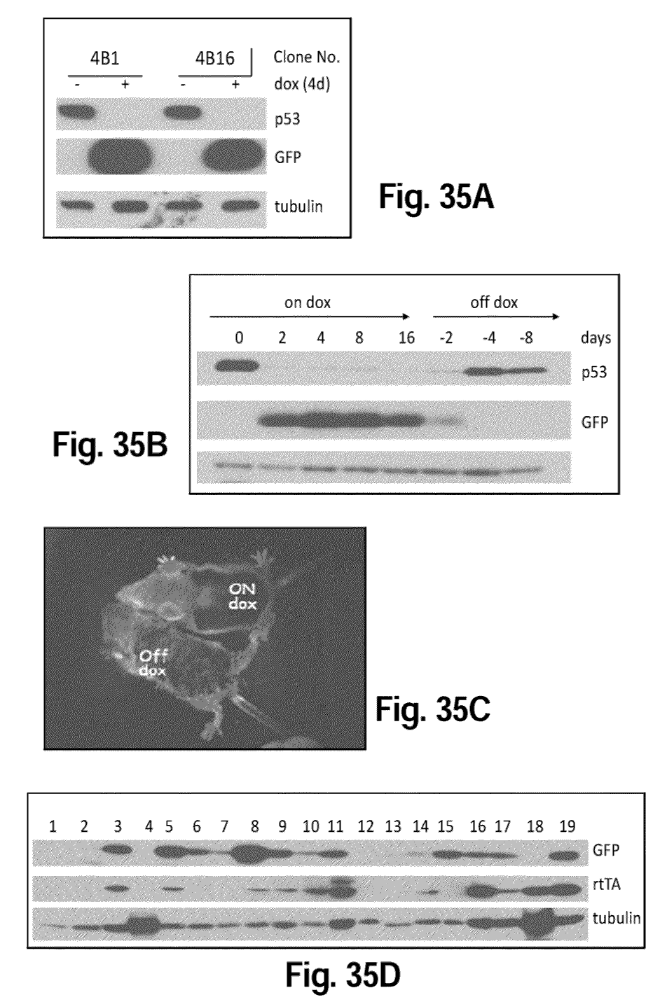

Generating the ColA1-TGMp16/19.478 (CT-p16/19) mice: KH2 ES cells, the pBS31 flp-in vector, and a pCAGGs-FLPe recombinase vector were obtained from the Jaenisch laboratory (Beard et al. 2006). Applicants modified the pBS31 flp-in vector, by inserting TRE-GFP-miR30 from the TGM vector (FIG. 34a). This new vector, pBS31-TGM, was also adapted to contain unique XhoI and EcoRI sites for rapid insertion of any shRNA into the miR30 backbone. Several shRNAs were then cloned into the pBS31-TGM vector including: p53.1224, p16/19.478, p16.117, p19.154 Luc.1309 (luciferase), and hRB.88 (human Rb). pBS31-TGMp16/19.478 and pCAGGs-FLPe were co-electroporated into KH2 ES cells to facilitate Frt-mediated recombination at the ColA1 locus. After correct integration of the pBS31-TGM vector, the cells became neomycin sensitive and hygromycin resistant (FIG. 34a). Additionally, since KH2 cells contain an rtTA (tet-on) cassette, surviving clones were treated with doxycycline to activate GFP expression, an indication that the integration was functional. The selection process was highly efficient: while there were only a few surviving hygromycin colonies, 100% of GFP positive clones screened thus far showed correct targeting by southern blot analysis (FIG. 34b). Further analyses by western blot of clones containing the p53.1224 shRNA demonstrated tight, regulatable knockdown correlating with GFP expression in cells treated with doxycycline (FIGS. 35a,35b). Western blot analysis was not possible for clones containing the p16/19.478 shRNA because ES cells do not express p16 or p19; accordingly, siNorthern analysis will be used to demonstrate shRNA expression in these clones. Following functional analysis of the ES cells in vitro, targeted clones were submitted to the mouse facility for tetraploid embryo complementation using standard protocol.

Functional analysis of CT-p16/19 derived MEFs: Mice produced by tetraploid embryo complementation were derived entirely from the targeted ES cells, thus with the presence of Rosa26-rtTA, which has been shown to have high expression in the skin, live mice can immediately be examined under the GFP-scope.

Two-week old mice were given doxycycline in their drinking water and, as anticipated, GFP expression was observed in their skin (FIG. 35c). Of the 8 pups that survived to 1 month of age, all contained the TGM-p16/19 cassette, as seen by PCR (data not shown). Further analyses of these targeted mice demonstrate robust GFP expression in several tissues by Western blot after 4 days of doxycycline treatment (FIG. 35d). While GFP expression should correlate with siRNA expression, further siNorthern analysis of GFP positive tissues may be used to directly verify siRNA expression in our RNAi mouse model. In the case that GFP does not precisely correlate with siRNA production in a specific tissue, it may suggests that the microRNA processing machinery may not be equally efficient in all tissue types.

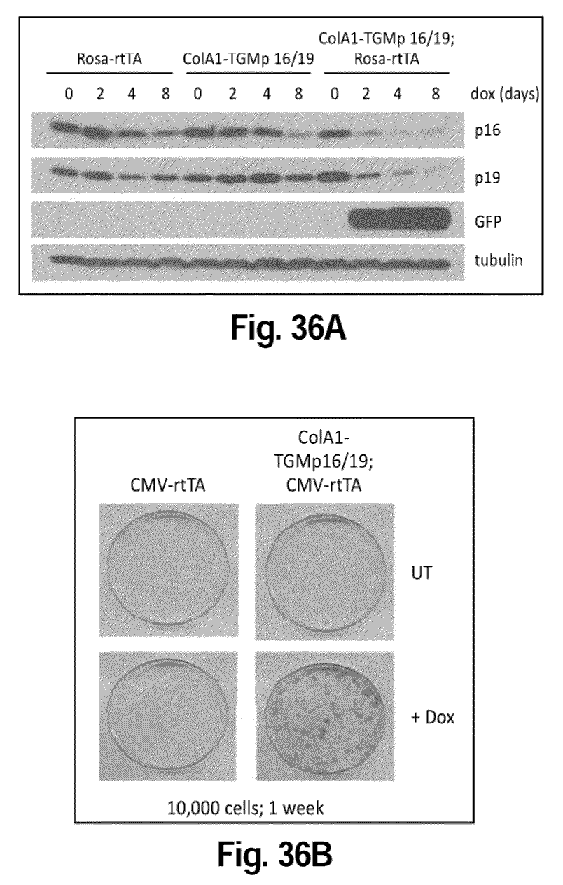

To demonstrate the ability of these mice to express the p16/19.478 shRNA from the TRE promoter, we isolated mouse embryonic fibroblasts (MEFs) from a cross between ColA1-TGM-p16/19.478 (referred to as CT-p16/19 from this point forward);Rosa-rtTA and wild-type C57BL/6 mice. MEFs of the correct genotype, determined by PCR, were treated with doxycycline and tested for p16 and p19 knockdown by Western blot (FIG. 36a). Consistent with the known effects of p19ARF deficiency, CT-p16/19 MEFs expressing rtTA formed colonies when plated at low density in the presence doxycycline but grew similarly to control MEFs without treatment (FIG. 36b).

To demonstrate that p16 and p19 knockdown is sufficient to cause tumor formation in vivo, CT-p16/19;Rosa-rtTA bitransgenic MEFs continuously treated with doxycycline will be infected with the Ras oncogene using standard protocols. Following selection for Ras integration, these MEFs will be subcutaneously injected into the flanks of immunocompromised nude mice, which have been pre-treated with doxycycline. Tumor formation will provide additional validation that the p16/19.478 shRNA causes sufficient knockdown to bypass Ras-induced senescence and cause transformation in MEFs. These tumors can be harvested and immunoblotted for GFP, p16 and p19 to further demonstrate that GFP expression correlates with protein knockdown in this system.

Functional analysis of ColA1-TGMp16/19.478 mice with various transgenic tTA lines: Many mouse lines have been developed that express the tTA (tet-off) or rtTA (tet-on) transactivators in different cell types, and these can be crossed to the ColA1-TGM mice to generate bitransgenic mice with regulatable, tissue-specific knockdown. Applicants have already generated many of these, including: Eμ-tTA, MMTV-tTA, LAP-tTA (liver specific), vav-tTA (pan-hematopoetic), CMV-rtTA and actin-rtTA. Applicants have already shown a Western blot of global GFP expression using Rosa-rtTA. The same analysis can be done using CMV-rtTA and actin-rtTA, or any other rtTA. GFP expression and siRNA production depend not only on the accessibility of gene expression at the ColA1 locus, but also on the availability of tTA or rtTA to induce transcription. CMV and the actin promoters may be stronger promoters than Rosa26, and thus they may promote higher levels of GFP and siRNA expression.

To further characterize the ColA1-TGM system, the mice may be crossed to Eμ-tTA, MMTV-tTA, LAP-tTA, and vav-tTA mice and test for GFP expression by Western blots and siRNA production by siNorthern in the B-cell compartment, mammary tissue, hepatocytes, and hematopoeitic compartment respectively.

Generating additional ColA1-TGM mice: To provide additional tools necessary for the experiments below, established methodology may be used to generate mice expressing the following shRNAs: p16.117, p19.154, Luc.1309, and hRb.88. In many tumor models, the entire INK4a/ARF locus is disrupted (D'Amico et al. 2003; Calvano, Rush et al. 1997; Serrano 2000; Kim and Sharpless 2000), giving rationale for using an shRNAmir targeting the entire locus (p16/19.478). However, regarding later therapeutical applications, it may be crucial to determine the therapeutic efficiency of restoration of endogenous levels of p19 or p16 alone. Thus the CT-p16.117 and CT-p19.154 mice can be used in concert with the CT-p16/19 mice to unravel the complexity of the INK4a/ARF locus.

To control for off-target effects of RNAi, it may be helpful to use two or more independent shRNAs against the same target. Furthermore, knockdown of the respective target will be documented. Thus where possible, two or more independent shRNAmir transgenics may be generated against every target gene and the results from each are compared.

In the instant case, in vitro studies have been conducted using multiple shRNAmirs to target p19 and p16/19, and no off-target effects have been noted.

Another potential advantage of the system is that it helps to avoid any potential RNAi toxicity (e.g., caused by overexpression of exogenous miRNAs that overwhelms the RNAi machinery, which tends to be most pronounced in the liver. See Grimm, Streetz et al. 2006). In the published study, the authors used Adeno-associated viruses to express shRNAs in the livers of mice. At the used multiplicity of infections (MOIs), each hepatocyte was infected with many AAV particles, resulting in nonphysiologically high shRNA expression in those cells. In contrast, the subject shRNAmir transgenic mice technology uses single copy integration of shRNAmir in the genome, and is unlikely to have the same RNAi toxicity issue seen with the AAV study.

As proper controls, CT-Luc.1309 and CT-hRb.88 mice may be generated, which mice express shRNAmirs targeting the luciferase transcript and human Rb transcript, respectively, neither of which exist in mice. These mice will be crossed in concert with the CT-p16/19 mice and will serve as a control cohort that produces shRNAmirs but no functional knockdown.

2: To Investigate the Effects of INK4a/ARF Re-Expression in Mouse Cancer Models

Inactivation of the INK4a/ARF locus is one of the most common occurrences in various human tumors, arising from homozygous deletions (14%), point mutations (5%), or promoter methylation (20%) (Sharpless 2005). In many cases, inactivation of INK4a/ARF is due to promoter methylation, such that these genes are not deleted but rather silenced; thus reactivation of these genes may be possible through the use of DNA demethylases (DNMTs) or histone deacetylase inhibitors (HDACis), which have emerged as a promising class of anti-neoplastic agents (Matheu, Klatt et al. 2005). While numerous studies have underscored the importance of INK4a/ARF loss in tumor development, no studies to date have determined the impact of p16 and ARF reactivation on tumor maintenance.

The subject regulatable RNAi mouse model can be used to study the effects of INK4a/ARF reactivation in cancer mouse models. As tumor genetics and biology may differ between hematopoietic malignancies and solid tumors, the consequences of p16/p19 reactivation may be studied in parallel in: 1) the Eμ-myc lymphoma model, and 2) the MMTV-myc mammary carcinoma model.

The Eμ-myc lymphoma model offers several benefits: 1) disease on-set and progression can be monitored by palpation of peripheral lymph nodes and blood smears; 2) lymphocytes can be repeatedly and non-invasively sampled throughout the animal's lifespan; 3) the tumors are transplantable; and 4) clonal signatures can be traced through Ig genetic rearrangements. It is expected that tumors will regress upon INK4a/ARF restoration, as seen previously with p53 restoration (Martins et al. 2006; Dickins et al. 2007). However, it is of interest to determine whether restoration will cause complete tumor remissions or whether tumors will relapse as a more aggressive disease. Loss of INK4a/ARF has been shown to cause less genomic instability when compared to p53 inactivation (Schmitt et al. 1999), thus it is conceivable that complete remission could occur unlike with p53 loss.

Similarly, MMTV-myc mammary tumorigenesis is a tractable model in which INK4a/ARF inactivation is believed to also play a crucial role for tumorigenesis (D'Amico et al. 2003). This model may be used to elucidate the mechanisms that regulate the initiation and progression of this disease. It is expected that in the MMTV-myc overexpression model, loss of INK4a/ARF function will accelerate breast tumorigenesis and that reactivation of INK4a/ARF genes will decrease tumor growth, possibly causing tumor regression and complete remission.

If reactivation of INK4a/ARF genes causes regression of lymphomas and mammary tumors in mice, it would suggest that lymphoma and breast cancer patients with INK4a/ARF mutation or hypermethylation of the locus could potentially benefit from the development of non-toxic DNMT/HDAC inhibitors. The effects of p16 and p19 re-expression may be studied separately. While loss of p16 alone is known to be a common early event in breast malignancies (Zhang et al. 2006), Eμ-myc lymphomagenesis is not accelerated in a p16−/− background (reviewed in Lowe and Sherr 2003). Thus, p16 and p19 re-expression may be studied separately in the MMTV-myc mammary carcinoma model, while independent reactivation of p19 will be studied in the Eμ-myc lymphoma model.

Both p16 and p19 proteins signal into different downstream pathways, which may contain additional therapeutic targets. p16, for example, acts as a tumor suppressor by inhibiting cdk4/6-cyclinD. p19 acts as a tumor suppressor by inhibiting MDM2 dependent degradation of p53. As it is very likely that pharmacological therapies with DNMT/HDAC inhibitors will only partially reverse silencing of the INK4A/ARF locus, it may be desirable to combine DNMT/HDAC inhibitors with cdk inhibitors and MDM2 inhibitors.

With the subject regulatable mouse model, not only can we investigate the impact of loss of p16/p19 proteins, we can also gain insight into the specific effects of INK4a/ARF re-activation in different contexts, with important implications for future cancer therapies. Investigation of these two models is ideal because we can compare the outcome of INK4a/ARF reactivation in the presence of the same initiating oncogene but in two tumor types: hematopoietic vs. solid epithelial tumors.

Methods:

A. Eμt-myc lymphoma model: To explore the consequences of INK4a/ARF reactivation in the Eμ-myc mouse model, Eμ-tTA transgenic mice is used to regulate expression of the p16/19.478 shRNA. CT-p16/19 mice are crossed to Eμ-tTA mice to generate bi-transgenic mice with regulatable p16 and p19 knockdown in mature B-cells, and knockdown is examined in FACS sorted populations of white blood cells collected from peripheral blood. The double transgenic CT-p16/19;Eμ-tTA mice are interbred with Eμ-myc mice to generate “triple” transgenic mice that are monitored closely for disease on-set by blood smears and lymph node palpation for the generation of a Kaplan-Meier disease onset curve. Similarly, the CT-p19 and CT-hRb or CT-luc mice are crossed to Eμ-tTA, and bitransgenic mice subsequently crossed to Eμ-myc mice. As the disease progresses, cohorts of mice are placed on doxycycline while leaving a number of animals untreated to determine whether INK4a/ARF restoration prolongs survival. It is expected that the lymphomas will regress after INK4a/ARF reestablishment, and with an intact myc to p19ARF to p53 signaling pathway, lymphomas will undergo apoptosis following re-expression, as suggested by p53 restoration (Martins et al. 2006). Lymphoma tissue can be isolated from lymph nodes using standard protocol, and Western blot and immunohistochemistry can be used to verify that p16 and p19 are knocked down when expected in the absence of doxycycline and restored upon doxycycline treatment. Sections of regressing tumors can be used for TUNEL-staining and SA-β-gal staining to examine whether the mechanism of tumor remission involves apoptosis, senescence or components of both programs. It is expected that the in the Eu-myc model, the predominant response to restoration of p16/p19 reactivation is apoptosis. If, however, tumor remissions involve a contribution of the senescence program, it may be desirable to determine whether the innate immune system is involved in clearing the senescent tumor cells. See below under the MMTV-myc breast cancer model. As discussed above, the results from the CT-p19 mice will facilitate the dissection of these two pathways and suggest a possible mechanism for tumor regression.

If in principle remissions are seen after reactivation of p16/19, the system can be used to perform pulse reactivations of p16/p19 and p19 in order to identify the minimal reactivation time to induce remissions. These studies will yield important information regarding later drug therapies. In case that there is relapses in the system, it will also be interesting to determine whether the duration of the treatment has an impact on the relapse rate. In any case, relapsing tumors may be analyzed regarding the mechanism of relapse: it may be possible that the shRNAmir can become constitutively active (breakdown of the tet-system). In this case, the relapsed tumors would be GFP positive even in the presence of doxycycline. It is also possible that the tumors may have acquired another mutation that rendered them independent of INK4a/ARF reestablishment. Another conceivable possibility is the loss of Trp53 or even the endogenous INK4a/ARF locus itself. PCR based methods to detect LOH of the p53 locus and the INK4A/ARF locus may be used to distinguish such possibilities. Furthermore, protocols to sequence the p53 locus for hot-spot mutations are readily available.

One major advantage in using the Eμ-myc lymphoma model is the ability to expand and transplant the tumors into a large number of recipients. Many of the previous questions can be addressed by further examining tumors from moribund triple transgenic mice. These lymphomas will be harvested and transplanted into a cohort of either immunocompetent or immunocompromised mice by tail vein injection, thus allowing us to observe the disease in a larger number of animals.

B. MMTV-myc mammary tumor model: To examine the effects of loss of INK4a/ARF genes in the MMTV-myc model, CT-p16/19 mice are crossed to MMTV-tTA mice to generate bi-transgenic mice with regulatable p16 and p19 knockdown. Mammary tissue from CT-p16/19;MMTV-tTA mice are harvested and knockdown of p16 and p19 are assessed by Western blot and immunohistochemistry. Concurrently, control mice are established using the CT-luc or CT-hRb mice, and similar experiments are performed with the CT-p16 and CT-p19 mice. Subsequently, bi-transgenic mice are interbred with MMTV-myc mice to create a mammary tumor model that will be used to assess loss of function of INK4a/ARF proteins in the presence of myc overexpression. One potential caveat of this model is that the MMTV promoter must be activated by hormones and proteins released during late pregnancy and lactation periods, thus it will be necessary to set-up triple transgenic females in breeding pairs in order to generate mammary tumors, which may delay the latency for tumor development. Triple transgenic mice are monitored by palpation and caliper measurement for mammary tumor formation. Compared to MMTV-c-myc mice that have mammary tumor latency of 9-12 months (McCormack, Weaver et al. 1998), Applicants have already observed tumor formation in only 6 months, despite the fact that only a limited number of mice were present in a colony. It is expected that knockdown of INK4a/ARF will accelerate mammary tumorigenesis, as seen in INK4a/ARF null mice (Paramio, Segrelles et al. 2001). Once mammary tumors have formed, cohorts of mice are placed on doxycycline while leaving another cohort untreated, and the mice are monitored for tumor sizes by caliper measure. Pathologist can determine the histological grade and stage of the malignancy by TNM (tumor, node, metastasis) classification. On specimens from regressing tumors, TUNEL staining and SA-β-Gal staining may be performed in order to determine whether the predominant response to p16/p19 reactivation in this model is apoptosis or senescence or a mixed response containing components of both programs. If TUNEL test or SA-β-Gal staining are positive, the results can be confirmed using additional markers (e.g. IHC for cleaved caspase 3 for apoptosis and IF for HMGA1 to stain for senescent cells). If tumor remissions are observed, but it remains difficult to detect significant rates of apoptosis or senescence in the tumors, regressing tumors may be characterized regarding forms of alternative cell death mechanisms.

It is expected that upon p16/p19 restoration, the mammary tumors will regress through a mixed response of apoptosis and senescence, with senescence probably being the predominant mechanism. Cellular senescence has primarily been known as a permanent cell cycle arrest. Only recently it has been shown that the cellular senescence program is linked to an innate immune response. Applicants have shown that restoration of endogenous levels of p53 in Ras driven liver carcinomas lead to induction of cellular senescence and via secreted chemokines to locally attract innate immune cells, which subsequently attacked and clear senescent tumor cells (Xue et al. 2007). If a predominant senescence response is observed in regressing breast carcinomas upon p16/19 restoration, it may be helpful to investigate whether the senescence response triggers an immune reaction against the tumor cells (see Xue et al. 2007). Sections from regressing tumors can be stained used H&E staining. Pathologist can then examine these sections regarding infiltrating innate immune cells. Furthermore, immunofluorescence stainings for macrophages, NK-cells and neutrophils on the tumor sections may be performed to quantify the different types of innate immune cells. To functionally prove that an immune response against senescent breast cancer cells is involved in tumor clearance upon p16/p19 reactivation, breast cancer bearing mice can be treated with compounds that specifically inhibit innate immune cells. For example, Macrophage function will be inhibited by Gadolinium chloride, NK-cells and neutrophils can be inhibited by neutralizing antibodies. Mice treated with immune inhibitors can subsequently be set on doxycycline to restore endogenous levels of p16/19 and remission rates will be determined as described above. Investigating the extent to which the immune system is necessary for tumor clearance is important because the immune system can be modulated by drug therapy and the results could implicate the use of such drugs in combination with p16/19 reactivating compounds in breast cancer therapy. One example could be the use of Interferon-γ (NK-cell stimulation) together with DNMT/HDAC inhibitors (reversion of INK4A/ARF silencing). Along the same lines as mentioned for the Eμ-myc study, all reactivation experiments will be performed with CT-p16/p19 compound mice as well as CT-p16 and CT-p19 single transgenics.

In summary, the Eμ-myc lymphoma and MMTV-myc mammary tumor models represent ideal tools in which we can dissect the INK4a/ARF tumor suppressor gene signaling pathways to further understand the networks involved in tumor maintenance. By using two cancer models with the same initiating lesion, we can compare and contrast the responses to INK4a/ARF re-expression in hematopoietic and solid tumors. These experiments hold great promise to answer important questions regarding the therapeutic applicability of therapies aiming at restoration of p16/p19 expression in lymphomas and breast carcinomas. As our p16/p19, p16 and p19 restoration experiments can mimic drug treatments, they will help to prioritize and justify time- and cost-intensive drug development efforts. These experiments will also reveal the tumor's predominant response towards p16/p19 restoration. A deeper understanding of these mechanisms will provide additional possibilities for therapeutic intervention (e.g. immunostimulatory therapies in combination with p16/p19 reactivation therapies in breast carcinomas that show a senescence response towards p16/p19 reactivation).

References referred-to in this example are listed below:
Adams, J. M., Harris, A. W., Pinkert, C. A., Corcoran, L. M., Alexander, W. S., Cory, S., Palmiter, R. D. and Brinster, R. L. The c-myc oncogene driven by immunoglobulin enhancers induces lymphoid malignancy in transgenic mice. Nature. 318 (533-538).
Beard, C., Hochedlinger, K., Plath, K., Wutz, A., & Jaenisch, R. Efficient Method to Generate Single-Copy Transgenic Mice by Site-Specific Integration in Embryonic Stem Cells. Genesis. 44 (2006) 23-28.
Boxer, R. B., Jang, J. W., Sintasath, L. & Chodosh, L. A. Lack of sustained regression of c-MYC-induced mammary adenocarcinomas following brief or prolonged MYC inactivation. Cancer Cell. 6 (2004) 577-586.
Calvano, J. E., Rush, E. B., Tan, L. K., Rosen, P. P., Borgen, P. I., & Van Zee, K. J. Absence of p16 gene (CDKN2) deletions in microdissected primary breast carcinoma specimens. Ann Surg Oncol. 5 (1997) 416-420.
Campbell, P. A., Perez-Iratxeta, C., Andrade-Navarro, M. A. and Rudnicki, M. A. Oct4 Targets Regulatory Nodes to Modulate Stem Cell Function. PLOS. 2 (2007) e553.
D'Amico, M., Wu, K., Di Vizio, D., Reutens, A. T., Stahl, M., Fu, M., Albanese, C., Russell, R. G., Muller, W. J., White, M., Negass, A., Lee, H., DePinho, R. A., & Pestell, R. The Role of Ink4a/Arf in ErbB2 Mammary Gland Tumorigenesis. Cancer Research. 63 (2003) 3395-3402.
Chin, L., Tam, A., Pomerantz, J., Wong, M., Holash, J., Bardessy, N., Shen, O., O'Hagan, R., Pantiginis, J., Zhou, H., Horner, J. W., Cordon-Cardo, C., Yancopoulos, G. D. and DePinho R. A. Essential role for oncogenic Ras in tumour maintenance. Nature. 400 (1999) 468-472.
Dickins, R. A., Hemann, M. T., Zilfou, J. T., Simpson, D. R., Ibarra, I., Hannon, G. J., & Lowe, S. W. Probing tumor phenotypes using stable and regulated synthetic microRNA precursors. Nature Genetics. 37 (2005) 1289-95.
Dickins, R. A., McJunkin, K., Hernando, E., Premsrirut, P. K., Krizhanovsky, V., Burgess, D. J., Kim, S. Y., Cordon-Cardo, C., Zender, L., Hannon, G. J. and Lowe, S. W. Tissue-specific and reversible RNAi in transgenic mice. Nature Gen. (in print).
Felsher, D. W. & Bishop, J. M. Reversible tumorigenesis by MYC in hematopoietic lineages. Mol. Cell. 4 (1999) 199-207.
Fisher, G., Wellen, S., Klimstra, D., Lenczowski, J., Tichelaar, J., Lizak, M., Whitsett, J., Koretsky, A., Varmus, H. Induction and apoptotic regression of lung adenocarcinomas by regulation of K-Ras transgene in the presence and absence of tumor suppressor genes. Gen Dev. 15 (2001) 3249-3262.
Furth, P. A., St. Onge, L., Boger, H., Gruss, P., Gossen, M., Kistner, A., Bujard, H. & Hennighausen, L. Temporal control of gene expression in transgenic mice by a tetracycline-responsive promoter. Proc. Natl. Acad. Sci. 27 (1994) 9302-9306.
Gray, M. D., Jesch, S. A. & Stein, G. H. 5-Azacytidine-induced demethylation of DNA to senescent level does not block proliferation of human fibroblasts. J Cell Physiol. 149 (1991) 477-484.
Grimm, D., Streetz, K. L., Jopling, C. L., Storm, T. A., Pandey, K., Davis, C. R., Marion, P., Salazar, F. and Kay, M. A. Fatality in mice due to oversaturation of cellular microRNA/short hairpin RNA pathways. Nature. 441 (2006) 381-405.
Hanahan, D. & Weinberg, R. A. The Hallmarks of Cancer. Cell. 100 (2000) 57-70.
Helmrich, A., Lee, S., O'Brien, P., Dorken, B., Lowe, S. W., Schrock, E. And Schmitt, C. A. Recurrent chromosomal aberrations in INK4a/ARF defective primary lymphomas predic drug responses in vivo. Oncogene. 24 (2005) 4174-4182.
Henningausen, L. Mouse models for breast cancer. Oncogene. 19 (2000) 966-967.
Hennighausen, L., Wall, R J., Tillmann, U., Li, M., Furth, P A. Conditional gene expression in secretory tissues and skin of transgenic mice using the MMTV-LTR and the tetracycline responsive system. J Cell Biochem. 59 (1995) 463-472.
Hruska, K. S., Tilli, M. T., Ren, S., Cotarla, I., Kwong, T., Li, M., Fondell, J. D., Hewitt, J. A., Koos, R. D., Furth, P. A. and Flaws, J. A. Conditional over-expression of estrogen receptor alpha in a transgenic mouse model. Trans Res. 11 (2002) 361-72.
Jonkers, J. & Berns, A. Oncogene addiction: Sometimes a temporary slayery. Cancer Cell. 6 (2004) 535-538.
Kim, W. Y. and Sharpless, N. E. The regulation of INK4/ARF in cancer and aging. Cell. 127 (2006) 265-275.
Lowe, S. W. and Sherr, C. J. Tumor suppression by Ink4a-Arf: progress and puzzles. Curr Op Gen Dev. 13 (2003) 77-83.
Martins, C. P., Brown-Swigart, L. and Evan, G. I. Modeling the Therapeutic Efficacy of p53 Restoration in Tumors. Cell. 127 (2006), 1323-1334.
Matheu, A., Klatt, P. and Serrano, M. Regulation of the INK4a/ARF locus by histone deacetylase inhibitors. JBC. 280 (2005) 42433-42441.
McCormack, S. J., Weaver, Z., Deming, S., Natarajan, G., Torri, J., Johnson, M. D., Liyanage, M., Ried, T. & Dickson, R. B. Myc/p53 interactions in transgenic mouse mammary development, tumorigenesis and chromosomal instability. Oncogene. 16 (1998) 2755-2766.
McCreath, K. J., Howcroft, J., Campbell, K. H., Colman, A., Schnieke, A. E. and Kind A. J. Production of gene-targeted sheep by nuclear transfer from cultured somatic cells. Nature. 405 (2000) 1066-1099.
Niwa, H., Miyazaki, J. and Smith, A. Quantitative expression of Oct-3/4 defines differentiation, dedifferentiation or self-renewal of ES cells. Nat. Gen. 24 (2000) 372-376.
Paramio, J. M., Segrelles, C., Ruiz, S., Martin-Caballero, J., Page, A., Martinez, J., Serrano, M. & Jorcano, J. L. The ink4a/arf tumor suppressors cooperate with p21cip1/waf in the processes of mouse epidermal differentiation, senescence, and carcinogenesis. J Biol Chem. 276 (2001) 44203-11.
Schuster-Gossler, K., Lee, A. W., Lerner, C. P., Parker, H. J., Dyer, V. W., Scott, V. E., Gossler, A. & Conover, J. C. Use of coisogenic host blastocyts or efficient establishment of germline chimeras with C57BL/6J ES cell lines. Biotechniques. 31 (2001) 1022-1024.
Schmitt C. A., McCurrach M. E., de Stanchina E., Wallace-Brodeur R. R. and Lowe S. W. INK4a/ARF mutations accelerate lymphomagenesis and promote chemoresistance by disabling p53. Genes Dev., 13 (1999) 2670-2677.
Serrano, M. The INK4a/ARF locus in murine tumorigenesis. Carcinogenesis. 21 (2000) 865-869.
Sharpless, N. E. INK4a/ARF: A multifunctional tumor suppressor locus. Mutation Research. 576 (2005) 22-38.
Sherr, C. J. Parsing Ink4a/Arf: “Pure” p16-Null Mice. Cell. 106 (2001) 531-534.
Siebler, J., Kleinridders, A., Kuter-Luks, B., Niehaves, S., Bruning, J., and Schwenk, F. Reversible gene knockdown in mice using a tight, inducible shRNA expression system. Nuc Acid Res. 35 (2007) e54.
Siebler, J., Zevnik, B., Kuter-Luks, B., Andreas, S., Kern, H., Hennek, T., Rode, A., Heimann, C., Faust, N., Kauselmann, G., Schoor, M., Jaenisch, R., Rajewsky, K., Kuhn, R. & Schwenk, F. Rapid generation of inducible mouse mutants. Nucl. Acids Res. 31 (2003) el 2.
Silva, J., Dominguez, G., Silva, J. M., Garcia, J., Gallego, I., Corbacho, C., Provencio, M., Espana, P. & Bonilla, F. Analysis of genetic and epigenetic processes that influence p14ARF expression in breast cancer. Oncogene. 20 (2001) 4586-4590.
Stegmeier, F., Hu, G., Rickles, R. J, Hannon, G. J., Elledge, S. J. A lentiviral microRNA-based system for single-copy polymerase II-regulated RNA interference in mammalian cells. PNAS. 102 (2005) 13212-13217.
Vogelstein, B. & Kinzler, K. W. Cancer genes and the pathways they control. Nature Medicine. 10 (2004) 789-799.
Ventura, A., Kirsch, D. G., McLaughlin, M. E., Tuveson, D. A., Grimm, J., Lintault, L., Newman, J., Reczek, E. E., Weissleder, R. and Jacks, T. Restoration of p53 function leads to tumour regression in vivo. Nature. 445 (2007) 661-665.
Wagner, K.-U., Wall, R. J., St-Onge, L., Gruss, P., Garrett, L., Wynshaw-Boris, A., Li, M., Furth, P. A. & Hennighausen, L. Cre mediated gene deletion in the mammary gland. Nucleic Acids. Res. 25 (1997) 4323-4330.
Weinstein, I. B. Addiction to Oncogenes—the Achilles Heal of Cancer. Science. 297 (2002) 63-64.
Weinstein, I. B. Disorders in cell circuitry during multistage carcinogenesis: the role of homeostasis. Carcinogenesis. 21 (2000) 857-864.
Wu, C., Riggelen, J., Yetil, A., Fan, A., Bachireddy, P. and Felsher, D. Cellular senescence is an important mechanism of tumor regression upon c-Myc inactivation. PNAS. 104 (2007) 13028-13033.
Xu, X., Li, C., Garrett-Beal, L., Larson, D., Wynshaw-Boris, A. & Deng, C. X. Direct removal in the mouse of a floxed neo gene from a three-loxP conditional knockout allele by two novel approaches. Genesis. 1 (2001) 1-6.
Xu, X., Wagner, K-U., Larson, D., Weaver, Z., Li, C., Ried, T., Hennighausen, L., Wynshaw-Boris, A. and Deng, C-X. Conditional mutant of Brcal in mammary epithelial cells results in blunted ductal morphogenesis and tumor formation. Nature Genetics. 22 (1999) 37-43.
Xue, W., Zender, L., Miething, C., Dickins, R. A., Hernando, E., Krizhanovsky, V., Cordon-Cardo, C. and Lowe, S. W. Senescence and tumor clearance is triggered by p53 restoration in murine liver carcinomas. Nature. 445 (2007) 656-660.
Zender, L., Zuber, J. and Lowe, S. W. Snapshot: genetic mouse models of cancer. Cell. 129 (2007) 838.
Zhang, J., Pickering, C., Holst, C., Gauthier, M., and Tlsty, T. p16INK4a modulates p53 in primary human mammary epithelial cells. Cancer Res. 66 (2006) 10325-10331.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

We claim:

1. A mammalian cell comprising a stably-integrated polynucleotide encoding an RNAi construct, wherein the expression of the RNAi construct is conditional.

2. The mammalian cell of claim 1, wherein the mammalian cell is a stem cell.

3. The mammalian cell of claim 1, wherein the mammalian cell is an embryonic stem cell.

4. The mammalian cell of claim 1, wherein the mammalian cell is a hematopoietic stem cell.

5. The mammalian cell of claim 1, wherein the mammalian cell is a human or mouse cell.

6. The mammalian cell of claim 1, wherein the mammalian cell is in culture.

7. The mammalian cell of claim 1, wherein the polynucleotide encoding the RNAi construct is a plasmid.

8. The mammalian cell of claim 1, wherein the polynucleotide encoding the RNAi construct is a retroviral vector or a lentiviral vector.

9. The mammalian cell of claim 1, wherein the polynucleotide encoding the RNAi construct is stably integrated into a defined locus of the genome.

10. The mammalian cell of claim 1, wherein a single copy of the polynucleotide encoding the RNAi construct is stably integrated into a defined locus of the genome.

11. The mammalian cell of claim 1, wherein the polynucleotide encoding the RNAi construct is stably integrated into a defined locus of the genome via Cre-mediated recombination.

12. The mammalian cell of claim 1, wherein the polynucleotide encoding the RNAi construct is stably integrated into a defined locus of the genome via FLP/FRT-mediated recombination.

13. The mammalian cell of claim 12, wherein the defined locus is ColA1.

14. The mammalian cell of claim 1, wherein the RNAi construct is short hairpin RNA (shRNA) or microRNA (miRNA).

15. The mammalian cell of claim 1, wherein the miRNA is mir30-based.

16. The mammalian cell of claim 1, wherein the conditional expression of the RNAi construct is temporal, tissue-specific, inducible, and/or reversible.

17. The mammalian cell of claim 1, wherein the conditional expression of the RNAi construct is reversed by excision of the stably integrated polynucleotide encoding the RNAi construct.

18. The mammalian cell of claim 1, wherein the expression of the RNAi construct is conditional on the presence or absence of a substance administered to the mammalian cell.

19. The mammalian cell of claim 18, wherein the substance is tetracyclin or an analog thereof.

20. The mammalian cell of claim 18, wherein the conditional expression of the RNAi construct is reversed by adding or withdrawing the substance.

21. The mammalian cell of claim 1, wherein the polynucleotide encoding the RNAi construct comprises an inducible tetracyclin-responsive promoter or TRE.

22. The mammalian cell of claim 21, further comprising a coding sequence for a tet-transactivator protein (tTA) or a reverse tet-transactivator protein (rtTA).

23. The mammalian cell of claim 22, wherein the tTA or rtTA exhibits tissue-specific expression.

24. The mammalian cell of claim 1, wherein the polynucleotide encoding the RNAi construct comprises a coding sequence for a fluorescent protein, a drug resistant gene, a SIN LTR, and/or an Internal Ribosomal Entry Site (IRES).

25. The mammalian cell of claim 24, wherein the fluorescent protein is GFP.

26. The mammalian cell of claim 24, wherein the expression of the RNAi construct is coupled to the expression of the fluorescent protein.

27. The mammalian cell of claim 1, wherein the RNAi construct is complementary to a portion of a target gene in the mammalian cell, and, when expressed, the RNAi construct causes partial or complete loss of function of the target gene.

28. A non-human mammal derived from or produced by the embryonic stem cell of claim 3.

29. The non-human mammal of claim 28, wherein the mammal is produced by tetraploid embryo complementation using the embryonic stem cell of claim 3.

30. A non-human mammal comprising the mammalian cell of claim 1, or a further proliferation or differention progeny thereof.

31. The non-human mammal of claim 28, wherein the RNAi construct is complementary to a portion of a target gene in the non-human mammal, and wherein the target gene participates in a disease process in the non-human mammal.

32. The non-human mammal of claim 31, wherein the target gene is a tumor suppressor gene.

33. The non-human mammal of claim 30, wherein the mammalian cell is an autologous cell derived from the non-human mammal.

34. The non-human mammal of claim 28, which is a mouse.

35. A method for identifying a gene that affects the sensitivity of tumor cells to a chemotherapeutic agent, the method comprising:
(a) introducing into a subject a transfected stem cell comprising a stably-integrated polynucleotide encoding an RNAi construct,
wherein the RNAi construct is complementary to at least a portion of a target gene,
wherein the expression of the RNAi construct is conditional, and,
wherein the transfected stem cell exhibits decreased expression of the target gene upon expression of the RNAi construct, and gives rise to a transfected tumor cell in vivo;
(b) evaluating the effect of the chemotherapeutic agent on the transfected tumor cell.

36. The method of claim 35, wherein evaluating the effect of the chemotherapeutic agent on the transfected tumor cell comprises: administering the chemotherapeutic agent to the subject and measuring the quantity of tumor cells derived from the transfected stem cell.

37. The method of claim 36, further comprising comparing the quantity of tumor cells derived from the transfected stem cell to the quantity of tumor cells derived from the transfected stem cell in a control subject that has not received the chemotherapeutic agent.

38. A method of determining a function of a gene in a cell, comprising:
(a) introducing into the cell a stably-integrated polynucleotide encoding an RNAi construct which targets mRNA of the gene, wherein the expression of the RNAi construct is conditional;
(b) maintaining the cell under conditions in which the RNAi construct is expressed and RNA interference of the mRNA occurs;
(c) assessing the phenotype of the cell, or the phenotype of a non-human mammal comprising the cell, as compared to a control;
thereby identifying a function of the gene.

39. The method of claim 38, wherein the control is an identical cell maintained under conditions in which the RNAi construct is not expressed, or an identical non-human mammal comprising the cell maintained under conditions in which the RNAi construct is not expressed.

40. The method of claim 38, further comprising:
(d) maintaining the cell under conditions in which the RNAi construct is not expressed and RNA interference of the mRNA does not occur;
(e) assessing the phenotype of the cell, or the phenotype of a non-human mammal comprising the cell.

41. The method of claim 38, wherein the non-human mammal is a mouse.

42. A polynucleotide encoding an RNAi construct, wherein the expression of the RNAi construct is conditional.
